(19) United States
(12) Patent Application Publication
(10) Pub. No.: US 2024/0060087 A1
(43) Pub. Date: Feb. 22, 2024

(54) METHODS AND COMPOSITIONS FOR MODULATING THE INTERACTION BETWEEN ADENO-ASSOCIATED VIRUS (AAV) AND THE AAV RECEPTOR (AAVR) FOR ALTERED BIO-DISTRIBUTION OF AAV

(71) Applicants: MASSACHUSETTS EYE AND EAR INFIRMARY, Boston, MA (US); THE SCHEPENS EYE RESEARCH INSTITUTE, INC., Boston, MA (US)

(72) Inventors: Eric Zinn, Lynn, MA (US); Christopher Tipper, Cambridge, MA (US); Luk H. Vandenberghe, Weston, MA (US)

(21) Appl. No.: 18/271,250

(22) PCT Filed: Jan. 7, 2022
(86) PCT No.: PCT/US2022/011682

§ 371 Date: Jul. 7, 2023

Related U.S. Application Data
(60) Provisional application No. 63/135,307, filed on Jan. 8, 2021.

(51) Int. Cl. C12N 15/86; C07K 14/005

(52) U.S. Cl. CPC C12N 15/86; C07K 14/005; C12N 2750/14122; C12N 2750/14143; C12N 2750/14145

(57) ABSTRACT

This disclosure describes compositions and methods for altering the bio-distribution of adeno-associated viruses (AAVs) in subjects.

### CROSS-REFERENCE TO RELATED APPLICATIONS

This application claims the benefit of U.S. Provisional Patent Application No. 63/135,307 filed on Jan. 8, 2021, the entire contents of which are incorporated herein by reference.

### TECHNICAL FIELD

This disclosure generally relates to viruses, and, specifically, adeno-associated viruses (AAVs).

### BACKGROUND

The adeno-associated virus (AAV), when rendered replication-defective, can be used as a vector system for therapeutic gene transfer. AAV is composed of a protein shell called a capsid that encapsulates a single-stranded DNA. The minimal requirements for the DNA molecule to enable encapsulation are that the DNA must be single-stranded and it must include flanking inverted terminal repeats (ITRs) of AAV. The capsid structure is a large multi-protein assembly that forms a globular particle with 20 facets composed of three 60-protein monomers in the form of an icosahedral assembly. These monomers form the capsid proteins. There are three capsid proteins, VP1, VP2, and VP3, which have overlapping sequences. VP3 is the shortest protein and makes up the primary particle structure, i.e., the essential building block to form the icosahedral assembly. VP2 is a longer protein that wholly encompasses VP3 in its C-terminus and is extended on the N-terminus. Similarly, VP1 encompasses VP2 and VP3 at its C-terminus. While VP1 and VP2 are not required structurally to form the capsid, both are required for infectivity of AAV.

In general, the capsid is thought to be the primary determinant of infectivity and host-vector related properties such as adaptive immune responses, tropism, specificity, potency, and bio-distribution. Indeed, several of these properties are known to vary between natural and engineered AAV serotypes and variants. To date, however, there is no mechanistic understanding as to how these changes on the capsid functionally alter these properties, and therefore, there is no rational basis to engineer AAVs toward any level of control of these properties. For example, several therapeutic approaches use AAV via a systemic injection to target global musculature for neuromuscular disorders such as Duchenne Muscular Dystrophy (DMD). However, vectors used currently for these approaches such as AAV9 and rh74 all require high doses to ensure muscle targeting and the vector naturally homes to the liver as a primary target. However, liver tissue is not involved in DMD disease pathology.

In 2016, the research group of Jan Carette identified a protein called AAVR (also known as KIAA0319L) as an essential entry factor or receptor for many AAVs (Pillay et al., 2016, Nature, 530(7588):108-12). In 2019, two independent groups reported on the structural resolution on the parts of the AAV particle that interface with the AAVR receptor (referred to here as the AAVR footprint) (Meyer et al., 2019, Elife, 8 pii: e44707; Zhang et al., 2019, Nat. Microbiol., 4(4):675-682).

### SUMMARY

The present disclosure is based, at least in part, on the discovery that one can manipulate, interfere with, or disrupt binding of various AAV to certain specific amino acids at specific locations within the AAVR footprint to alter the bio-distribution of an AAV in a subject, e.g., by modulating, e.g., decreasing or increasing the AAV's transduction of liver cells, without completely inhibiting AAV binding to the liver cells or the ability of the AAV to transduce liver or other cells in a subject.

In one aspect, the disclosure provides methods of modulating the bio-distribution of an adeno-associated virus (AAV) to liver cells within a subject. Such methods include providing an unmodified AAV capsid protein in an AAV; and replacing at least one amino acid residue at one or more of positions Q263, S264, G265, A266, S267, N268, H271, N382, G383, S384, Q385, S446, R471, W502, T503, D528, D529, Q589, K706, or V708 in the capsid protein (numbered relative to the AAV2 capsid sequence (SEQ ID NO:1)) in the AAV with a different amino acid to modify binding between the AAV capsid protein and an AAV receptor (AAVR) on a liver cell in a manner sufficient to alter, but not completely inhibit, the AAV's ability to transduce the liver cell.

In some instances, the bio-distribution to or in the liver cell by the AAV is increased. In other instances, the bio-distribution to or in the liver cell by the AAV is decreased.

In some embodiments, replacing the at least one amino acid residue includes mutagenesis of a nucleic acid encoding the AAV capsid protein. In some instances, the replacing step results in a conservative amino acid substitution.

In another aspect, the disclosure provides methods of modulating the bio-distribution of an adeno-associated virus (AAV) to liver cells within a subject. Such methods include providing an unmodified AAV capsid protein in an AAV; and replacing at least one amino acid residue at one or more of positions S446, R471, or V708 in the capsid protein (numbered relative to the AAV2 capsid sequence (SEQ ID NO:1)) in the AAV with a different amino acid to modify binding between the AAV capsid protein and an AAV receptor (AAVR) on a liver cell in a manner sufficient to alter, but not completely inhibit, the AAV's ability to transduce the liver cell.

In some instances, the AAV's ability to transduce the liver cell is increased, while, in some instances, the AAV's ability to transduce the liver cell is decreased.

In some embodiments, the replaced amino acid residues are any one or more of S446N, S446R, R471A, R471S, V708T, or V708A (numbered relative to the AAV2 capsid sequence (SEQ ID NO:1)). In some embodiments, the replacing of the at least one amino acid residue comprises mutagenesis of a nucleic acid encoding the AAV capsid protein.

In still another aspect, the disclosure provides non-naturally occurring AAV capsid proteins that include an AAV capsid protein having an amino acid sequence that differs from a wild type, unmodified AAV capsid protein amino acid sequence in at least one amino acid residue at a position selected from the group consisting of position 446, 471, or 708 (numbered relative to the AAV2 capsid sequence (SEQ ID NO:1)), where the non-naturally occurring AAV capsid protein amino acid sequence provides an altered binding of the AAV capsid protein for a liver cell than does the wild type AAV sequence in a manner sufficient to alter, but not completely inhibit, the AAV's ability to transduce liver cells.

In some embodiments, the non-naturally occurring amino acid sequence comprises at least one of S446N, S446R, R471A, R471S, V708T, or V708A (numbered relative to the AAV2 capsid sequence (SEQ ID NO:1)).

In some instances, the binding of the non-naturally occurring AAV capsid protein for the liver is increased, e.g., when the AAV capsid includes an R at position 446, an A at position 471, or a Tat position 708 (numbered relative to AAV2 (SEQ ID NO:1)). In some instances, the binding of the non-naturally occurring AAV capsid protein for the liver is decreased, e.g., when the AAV capsid includes an S at position 446, an S at position 471, or an A at position 708 (numbered relative to AAV2 (SEQ ID NO:1)).

In yet another aspect, the disclosure provides methods of altering the liver targeting of an AAV in a subject. Such methods include administering an AAV that includes a non-naturally occurring AAV capsid protein as described herein to the subject. In some embodiments, the administration is intravenously. In some embodiments, the administration is repeated a plurality of times.

In one aspect, the present disclosure features methods of altering the bio-distribution of an adeno-associated virus (AAV) in a subject, the methods include modulating the affinity or avidity of an AAV to a liver cell by disrupting or interfering with binding of the AAV to an AAV receptor (AAVR) on a liver cell, wherein the disrupting or interfering involves at least one amino acid residue at one or more of positions 263-265, 267, 268, 271, 382-385, 446, 471, 502, 503, 528-529, 589, 706, and 708 (relative to the AAV2 capsid sequence (SEQ ID NO:1 (top sequence in FIG. 1))).

In some implementations, the disrupting or interfering comprises mutagenesis. In certain embodiments the disrupting or interfering comprises small molecule binding or a chemical or peptide modification of an AAV capsid protein.

In another aspect, the disclosure features non-naturally occurring AAV capsid proteins, including an AAV capsid having a recombinant amino acid sequence that differs from a wild type or unmodified sequence in at least one amino acid residue at a position selected from the group consisting of positions 263-265, 267, 268, 271, 382-385, 446, 471, 502, 503, 528-529, 589, 706, and 708 (relative to the AAV2 capsid sequence (SEQ ID NO:1 (top sequence in FIG. 1))), wherein the recombinant AAV amino acid sequence provides an altered affinity or avidity of the capsid protein for a liver cell than does the wild type AAV sequence.

In some implementations, the at least one amino acid residue that differs between the recombinant amino acid sequence and the wild type sequence is shown in Table 1.

In certain embodiments, the affinity or avidity of the non-naturally occurring AAV capsid protein for the liver is increased. In some implementations, the AAV capsid comprises a R at position 446 or a T at position 708 (relative to AAV2).

In certain embodiments, the affinity or avidity of the non-naturally occurring AAV capsid protein for the liver is decreased. In certain embodiments, the AAV capsid comprises an S at position 446 or an A at position 708 (relative to AAV2).

In another aspect, the disclosure features methods of altering the liver targeting of an AAV in a subject, the methods include administering an AAV comprising the non-naturally occurring AAV capsid protein described herein to the subject.

In yet another aspect, the disclosure features methods of altering the liver targeting of an AAV in a subject, the methods include administering an AAV comprising the non-naturally occurring AAV capsid protein described herein to the subject.

In one aspect, the disclosure provides viruses including a non-naturally occurring, modified AAV VP1 capsid protein. Typically, viruses as described herein include an amino acid sequence having at least 95% sequence identity to an amino acid sequence of an unmodified AAV VP1 capsid protein when the amino acid sequence of the modified AAV capsid protein and the amino acid sequence of the unmodified AAV VP1 capsid protein are aligned using a basic local alignment search tool (BLAST) program with default algorithm parameters; where the amino acid sequence of the modified VP1 capsid protein differs from the amino acid sequence of the unmodified VP1 capsid protein in at least one amino acid position selected from the group consisting of amino acid positions Q263, S264, G265, A266, S267, N268, H271, N382, G383, S384, Q385, S446, R471, W502, T503, D528, D529, Q589, K706, and V708, which are numbered relative to AAV2 VP1 capsid protein (SEQ ID NO:1) when SEQ ID NO:1 and the amino acid sequence of the unmodified AAV capsid protein are aligned using the basic local alignment search tool (BLAST) program with default algorithm parameters.

In some embodiments, the default parameters for BLASTP include: parameters automatically adjusted for short input sequences; expect threshold: 10; word size: 3; max matches in a query range: 0; matrix: BLOSUM62; gap costs: existence 11, extension 1; compositional adjustments: conditional compositional score matrix adjustment; and no filters or masks). In some embodiments, the default parameters for BLASTN are: parameters automatically adjusted for short input sequences; expect threshold: 10; word size: 28; max matches in a query range: 0; match/mismatch scores: 1,-2; gap costs: linear; filter: low complexity regions; and mask: for lookup table only.

In some embodiments, the amino acid sequence of the modified VP1 capsid protein includes at least one amino acid residue (e.g., at least two amino acid residues) selected from the group consisting of 446R, 471A, and 708T. In some embodiments, the amino acid sequence of the modified VP1 capsid protein comprises amino acid residues 446R, 471A, and 708T. In some embodiments, the amino acid sequence of the modified VP1 capsid protein comprises at least one amino acid residue (e.g., at least two amino acid residues) selected from the group consisting of 446S, 471S, and 708A. In some embodiments, the amino acid sequence of the modified VP1 capsid protein comprises 446S, 471S, and 708A.

In some embodiments, the amino acid sequence of the modified VP1 capsid protein differs from the amino acid sequence of the unmodified VP1 capsid protein only in one or more of the amino acid positions Q263, S264, G265, A266, S267, N268, H271, N382, G383, S384, Q385, S446, R471, W502, T503, D528, D529, Q589, K706, and V708, and in no other amino acid positions in the unmodified VP1 capsid protein.

In some embodiments, the unmodified VP1 capsid protein is selected from the group consisting of a VP1 capsid protein from AAV1, AAV2, AAV3, AAV6, AAV7, AAV8, AAV9, rh.10, hu.37, LK-03, AAV5, AAV10, Hu68; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; Anc80DI; AAV1 vp1; AAV2 vp1; AAV9vp1; Anc80; Anc126; Anc127; AAV3; AAV7; AAV8; rh10; hu37; and hu.68.

In certain embodiments, the non-naturally occurring, modified AAV VP1 capsid protein includes an amino acid sequence having at least 96%, 97%, 98% or 99% sequence identity to the amino acid sequence of the unmodified AAV VP1 capsid protein when the amino acid sequence of the modified AAV capsid protein and the amino acid sequence of the unmodified AAV VP1 capsid protein are aligned using a basic local alignment search tool (BLAST) program with default algorithm parameters.

In another aspect, the disclosure provides modified, assembly-competent recombinant AAVs (rAAVs). Such AAVs include VP1, VP2, and VP3 capsid proteins, and a recombinant nucleic acid vector, where the VP1 capsid protein is a modified VP1 capsid protein as described herein.

In still another aspect, the disclosure provides modified, assembly-competent recombinant AAVs (rAAVs). Such AAVs include VP1, VP2, and VP3 capsid proteins; and a recombinant nucleic acid vector, where at least the VP1 capsid protein is a non-naturally occurring, modified VP1 capsid protein comprising an amino acid sequence having at least 95% sequence identity to an amino acid sequence of an unmodified AAV VP1 capsid protein when the amino acid sequence of the modified AAV capsid protein and the amino acid sequence of the unmodified AAV VP1 capsid protein are aligned using a basic local alignment search tool (BLAST) program with default algorithm parameters, and where the modified VP1 capsid protein differs from the unmodified VP1 capsid protein in comprising a means for altering biodistribution of the modified rAAV following administration of the modified rAAV to a first mammalian subject as compared to biodistribution of an unmodified rAAV following administration of the unmodified rAAV having the unmodified VP1 capsid protein to a second mammalian subject of the same type as the first mammalian subject, wherein the unmodified rAAV comprises VP1, VP2, and VP3 capsid proteins having amino acid sequences identical to those of the modified rAAV except for said means.

In some embodiments, the modified rAAV achieves higher transduction of liver cells following administration to a first mammalian subject as compared to transduction of liver cells following administration of the unmodified rAAV including the unmodified VP1 capsid protein to a second mammalian subject of the same type as the first mammalian subject. In some embodiments, the modified rAAV exhibits higher expression in liver cells of an expressible polypeptide encoded by the recombinant nucleic acid vector following administration to a first mammalian subject as compared to expression in liver cells of the expressible polypeptide following administration of an unmodified rAAV including the unmodified VP1 capsid protein to a second mammalian subject of the same type as the first mammalian subject.

In some embodiments, the modified rAAV achieves lower transduction of liver cells following administration to a first mammalian subject as compared to transduction of liver cells following administration of an unmodified rAAV including the unmodified VP1 capsid protein to a second mammalian subject of the same type as the first mammalian subject. In some embodiments, the modified rAAV exhibits lower expression in liver cells of an expressible polypeptide encoded by the recombinant nucleic acid vector following administration to a first mammalian subject as compared to expression in liver cells of the expressible polypeptide following administration of an unmodified rAAV including the unmodified VP1 capsid protein to a second mammalian subject of the same type as the first mammalian subject.

In some embodiments, the modified rAAV has an altered interaction with an AAV receptor (AAVR) expressed on liver cells of the first mammalian subject as compared to an unmodified rAAV including the unmodified VP1 capsid protein with an AAVR expressed on liver cells of the second mammalian subject. In some embodiments, the modified rAAV has increased interaction with an AAV receptor (AAVR) expressed on liver cells of the first mammalian subject as compared to an unmodified rAAV including the unmodified VP1 capsid protein with an AAVR expressed on liver cells of the second mammalian subject. In some embodiments, the modified rAAV has decreased interaction with an AAV receptor (AAVR) expressed on liver cells of the first mammalian subject as compared to an unmodified rAAV including the unmodified VP1 capsid protein with an AAVR expressed on liver cells of the second mammalian subject.

In some embodiments, the first and second mammalian subjects are humans or non-human primates (NHP). In some embodiments, the administration comprises systemic administration, e.g., intravenous infusion. In some embodiments, the modified rAAV has lower liver toxicity when administered to a mammalian subject, e.g., a human subject, than an unmodified rAAV comprising the unmodified VP1 capsid protein administered in the same amount by the same route of administration.

In some embodiments, means for altering biodistribution of the modified rAAV following administration of the modified rAAV to a first mammalian subject includes a mutation at one or more amino acid residues at positions selected from the group consisting of Q263, S264, G265, A266, S267, N268, H271, N382, G383, S384, Q385, S446, R471, W502, T503, D528, D529, Q589, K706, and V708, which are numbered relative to an AAV2 VP1 capsid protein (SEQ ID NO:1) when SEQ ID NO:1 and the amino acid sequence of the unmodified AAV capsid protein are aligned using the basic local alignment search tool (BLAST) program with default algorithm parameters.

In some embodiments, the modified VP1 capsid protein includes at least one amino acid residue (e.g., at least two amino acid residues) selected from the group consisting of 446R, 471A, and 708T. In some embodiments, the modified VP1 capsid protein comprises amino acid residues 446R, 471A, and 708T.

In some embodiments, the modified VP1 capsid protein includes at least one amino acid residue (e.g., at least two amino acid residues) selected from the group consisting of 446S, 471S, and 708A. In some embodiments, the modified VP1 capsid protein includes amino acid residues 446S, 471S, and 708A.

In some embodiments, the amino acid sequence of the modified VP1 capsid protein differs from the amino acid sequence of the unmodified VP1 capsid protein only in one or more of the amino acid positions Q263, S264, G265, A266, S267, N268, H271, N382, G383, S384, Q385, S446, R471, W502, T503, D528, D529, Q589, K706, and V708, and in no other amino acid positions in the unmodified VP1 capsid protein.

In some embodiments, the unmodified VP1 capsid protein is selected from the group consisting of a VP1 capsid protein from AAV1, AAV2, AAV3, AAV6, AAV7, AAV8, AAV9, rh.10, hu.37, LK-03, AAV5, AAV10, Hu68; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; Anc80DI; AAV1 vp1; AAV2 vp1; AAV9vp1; Anc80; Anc126; Anc127; AAV3; AAV7; AAV8; rh10; hu37; and hu.68.

In some embodiments, the amino acid sequence of the modified VP1 capsid protein is at least 96% identical, 97% identical, 98% identical, or 99% identical to the amino acid sequence of the unmodified VP1 capsid protein.

In yet another aspect, pharmaceutical compositions are provided that include a modified rAAV as described herein, and a pharmaceutically acceptable carrier. In one aspect, nucleic acid molecules encoding a modified VP1 capsid protein as described herein or the VP1 protein of a modified rAAV as described herein.

In another aspect, vectors are provided that include a nucleic acid molecule as described herein. In another aspect, isolated host cells are provided that include a nucleic acid molecule as described herein or a vector as described herein.

In still another aspect, the disclosure provides methods of altering delivery of an expressible polynucleotide to a target organ of a mammalian subject (e.g., a human patient), e.g., as compared to delivery using an rAAV with an unmodified VP1 capsid protein. Such methods can include administering a therapeutically effective dose of a modified rAAV as described herein or a pharmaceutical composition as described herein to the human patient. In some embodiments, the expressible nucleic acid is a transgene.

In some embodiments, the modified rAAV exhibits higher transduction of an expressible polypeptide encoded by the recombinant nucleic acid vector into cells of the target organ following administration to a first mammalian subject as compared to transduction into cells of the target organ of the expressible polypeptide following administration of an unmodified rAAV comprising the unmodified VP1 capsid protein to a second corresponding mammalian subject.

In certain embodiments, the modified rAAV exhibits lower transduction of an expressible polypeptide encoded by the recombinant nucleic acid vector into cells of the target organ following administration to a first mammalian subject as compared to transduction into cells of the target organ of the expressible polypeptide following administration of an unmodified rAAV comprising the unmodified VP1 capsid protein to a second corresponding mammalian subject.

In some embodiments, the modified rAAV exhibits higher transduction of an expressible polypeptide encoded by the recombinant nucleic acid vector into cells outside of the target organ following administration to a first mammalian subject as compared to transduction into cells outside of the target organ of the expressible polypeptide following administration of an unmodified rAAV comprising the unmodified VP1 capsid protein to a second corresponding mammalian subject.

In some embodiments, the modified rAAV exhibits lower transduction of an expressible polypeptide encoded by the recombinant nucleic acid vector into cells outside of the target organ following administration to a first mammalian subject as compared to transduction into cells outside of the target organ of the expressible polypeptide following administration of an unmodified rAAV comprising the unmodified VP1 capsid protein to a second corresponding mammalian subject.

In certain embodiments, the modified rAAV exhibits higher expression of an expressible polypeptide encoded by the recombinant nucleic acid vector in cells in cells of the target organ following administration to a first mammalian subject as compared to expression in cells of the target organ of the expressible polypeptide following administration of an unmodified rAAV comprising the unmodified VP1 capsid protein to a second corresponding mammalian subject.

In some embodiments, the modified rAAV exhibits lower expression of an expressible polypeptide encoded by the recombinant nucleic acid vector in cells of the target organ following administration to a first mammalian subject as compared to expression in cells of the target organ of the expressible polypeptide following administration of an unmodified rAAV comprising the unmodified VP1 capsid protein to a second corresponding mammalian subject.

In some embodiments, the modified rAAV exhibits higher expression in cells outside of the target organ of an expressible polypeptide encoded by the recombinant nucleic acid vector following administration to a first mammalian subject as compared to expression in cells of the target organ of the expressible polypeptide following administration of an unmodified rAAV comprising the unmodified VP1 capsid protein to a second corresponding mammalian subject.

In certain embodiments, the modified rAAV exhibits lower expression in cells outside of the target organ of an expressible polypeptide encoded by the recombinant nucleic acid vector following administration to a first mammalian subject as compared to expression in cells of the target organ of the expressible polypeptide following administration of an unmodified rAAV comprising the unmodified VP1 capsid protein to a second corresponding mammalian subject.

In some embodiments, the target organ is the liver. In some embodiments, cells outside the target organ are muscle cells.

In some embodiments, the unmodified AAV is AAV1, AAV8, or AAV9, and the target organ, to which delivery is altered, is the heart; the unmodified AAV is AAV2, and the target organ, to which delivery is altered, is the kidney; the unmodified AAV is AAV7, AAV8, AAV9, and the target organ, to which delivery is altered, is the liver; the unmodified AAV is AAV4, AAV5, AAV6, AAV9, and the target organ, to which delivery is altered, is the lung; the unmodified AAV is AAV8, and the target organ, to which delivery is altered, is the pancreas; the unmodified AAV is AAV2, AAV5, AAV8, and the target organ, to which delivery is altered, is the photoreceptor cells of the eye; the unmodified AAV is AAV1, AAV2, AAV4, AAV5, AAV8, and the target organ, to which delivery is altered, is the Retinal Pigment Epithelium (RPE); and the unmodified AAV is AAV1, AAV6, AAV7, AAV8, AAV9, and the target organ, to which delivery is altered, is the skeletal muscle.

In some embodiments, the modified rAAV has lower liver toxicity when administered to a mammalian subject, e.g., a human subject, than the same dose of an unmodified rAAV comprising the unmodified VP1 capsid protein administered by the same route of administration. In some embodiments, the mammalian subject is a human subject or a non-human primate.

In still another aspect, the disclosure provides compositions for use in any of the methods described herein.

In one aspect, in a method of treating a mammalian subject (e.g., a human patient) by administering a recombinant AAV (rAAV), the improvement includes administering a therapeutically effective dose of a modified rAAV that comprises a capsid having means for altering rAAV biodistribution following administration to a mammalian subject.

In some embodiments, the means for altering rAAV biodistribution reduce liver clearance of the rAAV. In some embodiments, the means for altering rAAV biodistribution increase transduction of cells of a target organ. In some embodiments, the means for altering rAAV biodistribution increase expression in cells of a target organ of an expressible polypeptide encoded by the recombinant nucleic acid vector. In some embodiments, the means for altering rAAV biodistribution decrease transduction of cells of a target organ. In some embodiments, the means for altering rAAV biodistribution decrease expression in cells of a target organ of an expressible polypeptide encoded by the recombinant nucleic acid vector. In some embodiments, the means for altering rAAV biodistribution increase transduction of cells outside of a target organ.

In certain embodiments, the means for altering rAAV biodistribution increase expression in cells outside of a target organ of an expressible polypeptide encoded by the recombinant nucleic acid vector. In some embodiments, the means for altering rAAV biodistribution decrease transduction of cells outside of a target organ. In some embodiments, the means for altering rAAV biodistribution decrease expression in cells outside of a target organ of an expressible polypeptide encoded by the recombinant nucleic acid vector. In some embodiments, the means for altering rAAV biodistribution alter interaction of the modified rAAV with the AAVR expressed on cells of the mammalian subject. In some embodiments, the means for altering rAAV biodistribution reduce interaction of the modified rAAV with the AAVR. In some embodiments, the means for altering rAAV biodistribution increase interaction of the modified rAAV with the AAVR. In some embodiments, the modified rAAV has less liver toxicity than the unmodified rAAV.

In certain embodiments, the modified rAAV achieves a 10-fold reduction in gene transfer to liver cells after a first administration into a mammalian subject compared to an unmodified rAAV. In some embodiments, the modified rAAV achieves a 10-fold increase in gene transfer to liver cells after a first administration into a rhesus macaque compared to an unmodified rAAV.

In some embodiments, the modified rAAV achieves a 100-fold increase in gene transfer to liver cells following the first administration into a mammalian subject. In some embodiments, the modified rAAV achieves a 100-fold decrease in gene transfer to liver cells following the first administration into a mammalian subject.

In certain embodiments, the modified rAAV achieves a 1000-fold increase in gene transfer to liver cells following the first administration into a mammalian subject. In some embodiments, the modified rAAV achieves a 1000-fold decrease in gene transfer to liver cells following the first administration into a mammalian subject.

The term “mutation” refers to a change or alteration in the native or natural sequence or a nucleic acid or an amino acid. A mutation can be naturally occurring or a mutation can be engineered in the laboratory (e.g., man-made).

The term “unmodified AAV capsid protein” as used herein refers to a VP1, VP2, or VP3 capsid protein of a naturally occurring AAV serotype or a non-naturally occurring VP1, VP2, or VP3 capsid protein available or known in the art. The non-naturally occurring VP1, VP2, or VP3 capsid protein includes a capsid protein generated by biological or chemical alteration or variation of a naturally occurring AAV capsid protein. Accordingly, unmodified AAV capsid proteins include, but are not limited to, a capsid protein of various AAV serotypes (e.g., AAV1, AAV2, AAV3B, AAV5, AAV6, AAV8, and AAV9) or a variant thereof. As used herein, “variant” refers to a naturally occurring or artificially created relative of the indicated serotype available or known in the art. A non-naturally occurring VP1, VP2, or VP3 capsid protein further includes an artificial capsid protein created by in silico design or synthesis. An artificial capsid protein includes, but is not limited to, AAV capsid proteins disclosed in PCT/US2014/060163, U.S. Pat. No. 9,695,220, PCT/US2016/044819, PCT/US2018/032166, PCT/US2019/031851, and PCT/US2019/047546, which are incorporated herein by reference in their entireties.

Representative unmodified AAV capsid proteins can be VP1, VP2 or VP3 capsid proteins of an AAV selected from, without limitation, AAV2 (SEQ ID NO:1); rh. 58-E (SEQ ID NO:2); rh. 40-E (SEQ ID NO:3); AAV1 (SEQ ID NO:4); AAV6 (SEQ ID NO:5); AAV3 (SEQ ID NO:6); AAV LK03 (SEQ ID NO:7); AAV7 (SEQ ID NO:8); AAV8 (SEQ ID NO:9); AAV hu.37 (SEQ ID NO:10); AAV rh.10 (SEQ ID NO:11); AAV9 (SEQ ID NO:12); AAV hu.68 (SEQ ID NO:13); AAV10 (SEQ ID NO:14); AAV5 (SEQ ID NO:15); AAV3-3 (SEQ ID NO:16); AAV4-4 (SEQ ID NO:17); AAV1-A (SEQ ID NO:18); hu. 46-A (SEQ ID NO:19); hu. 48-A (SEQ ID NO:20); hu. 44-A (SEQ ID NO:21); hu. 43-A (SEQ ID NO:22), AAV6-A (SEQ ID NO:23); hu. 34-B (SEQ ID NO:24), hu. 47-B (SEQ ID NO:25), hu. 29-B (SEQ ID NO:26), rh. 63-B (SEQ ID NO:27), hu. 56-B (SEQ ID NO:28), hu. 45-B (SEQ ID NO:29), rh. 57-B (SEQ ID NO:30), rh. 35-B (SEQ ID NO:31); rh. 58-B (SEQ ID NO:32); rh. 28-B (SEQ ID NO:33); rh. 51-B (SEQ ID NO:34); rh. 19-B (SEQ ID NO:35); rh. 49-B (SEQ ID NO:36), rh. 52-B (SEQ ID NO:37); rh. 13-B (SEQ ID NO:38); AAV2-B (SEQ ID NO:39); rh. 20-B (SEQ ID NO:40); rh. 24-B (SEQ ID NO:41); rh. 64-B (SEQ ID NO:42); hu. 27-B (SEQ ID NO:43); hu. 21-B (SEQ ID NO:44); hu. 22-B (SEQ ID NO:45); hu. 23-B (SEQ ID NO:46); hu. 7-C(SEQ ID NO:47); hu. 61-C(SEQ ID NO:48); rh. 56-C(SEQ ID NO:49); hu. 9-C(SEQ ID NO:59); hu. 54-C(SEQ ID NO:51); hu. 53-C(SEQ ID NO:52); hu. 60-C(SEQ ID NO:53); hu. 55-C(SEQ ID NO:54); hu. 2-C (SEQ ID NO:55); hu. 1-C(SEQ ID NO:56); hu. 18-C(SEQ ID NO:57); hu. 3-C(SEQ ID NO:58); hu. 25-C(SEQ ID NO:59); hu. 15-C(SEQ ID NO:60); hu. 16-C(SEQ ID NO:61); hu. 11-C(SEQ ID NO:62); hu. 10-C(SEQ ID NO:63); hu. 4-C(SEQ ID NO:64); rh. 54-D (SEQ ID NO:65); rh. 48-D (SEQ ID NO:66); rh. 55-D (SEQ ID NO:67); rh. 62-D (SEQ ID NO:68); AAV7-D (SEQ ID NO:69); rh. 52-E (SEQ ID NO:70); rh. 51-E (SEQ ID NO:71); hu. 39-E (SEQ ID NO:72); rh. 53-E (SEQ ID NO:73); hu. 37-E (SEQ ID NO:74); rh. 43-E (SEQ ID NO:75); rh. 50-E (SEQ ID NO:76); rh. 49-E (SEQ ID NO:77); rh. 61-E (SEQ ID NO:78); hu. 41-E (SEQ ID NO:79); rh. 64-E (SEQ ID NO:80); hu. 42-E (SEQ ID NO:81); rh. 57-E (SEQ ID NO:82); rh. 40-E (SEQ ID NO:83); hu. 67-E (SEQ ID NO:84); hu. 17-E (SEQ ID NO:85); hu. 6-E (SEQ ID NO:86); hu. 66-E (SEQ ID NO:87); rh. 38-E (SEQ ID NO:88); hu. 32-F (SEQ ID NO:89); AAV9/hu (SEQ ID NO:90); hu. 31-F (SEQ ID NO:91); Anc80 (SEQ ID NO:92); Anc81 (SEQ ID NO:93); Anc82 (SEQ ID NO:94); Anc83 (SEQ ID NO:95); Anc84 (SEQ ID NO:96); Anc94 (SEQ ID NO:97); Anc113 (SEQ ID NO:98); Anc126 (SEQ ID NO:99); Anc127 (SEQ ID NO:100); Anc80L27 (SEQ ID NO:101); Anc80L59 (SEQ ID NO:102); Anc80L60 (SEQ ID NO:103); Anc80L62 (SEQ ID NO:104); Anc80L65 (SEQ ID NO:105); Anc80L33 (SEQ ID NO:106); Anc80L36 (SEQ ID NO:107); Anc80L44 (SEQ ID NO:108); Anc80L1 (SEQ ID NO:109); Anc110 (SEQ ID NO:110); Anc80DI (SEQ ID NO:111) and AAV8-E (SEQ ID NO:153).

The term “unmodified rAAV” as used herein refers to a recombinant AAV (rAAV) comprising only unmodified AAV capsid proteins.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a schematic showing a minimal design of a library construct for AAV variants within a pooled barcoded library. ITR, Inverted Terminal Repeat; polyA, poly adenylation signals; ORF, open reading frame.

FIG. 2 is an alignment of Anc126 (SEQ ID NO:99) and Anc127 (SEQ ID NO:100) VP1 capsid protein sequences relative to the AAV2 capsid protein sequence (SEQ ID NO:1). The residues in the AAV2 capsid sequence predicted to interact with the AAVR footprint (the AAVR binding domain of the AAV capsid protein sequence) are boxed.

FIG. 3 is a fingerprint plot that illustrates relative rank of Anc126 variants in terms of murine liver targeting. Each Anc126 variant was ranked from top to bottom based on liver uptake following IV administration of a multiplex Anc126 library. Columns show the nine positions of variation that make up the diversity within Anc126. Each position of variation has two possible residues, represented here as black or white. The percent of variants with that particular residue (i.e., black) in either the top or bottom half rank-order is indicated in the accompanying table.

FIG. 4 is a fingerprint plot that illustrates relative rank of Anc127 variants in terms of murine liver targeting. Each Anc127 was ranked from top to bottom based on liver uptake following IV administration of a multiplex Anc127 library. Columns show the ten positions of variation that make up the diversity within Anc127. Each position of variation has two possible residues, represented here as black or white. The percent of variants with that particular residue (i.e., black) in either the top or bottom half rank-order is indicated in the accompanying table.

FIGS. 5A-5B are fingerprint plots that illustrate relative rank order of Anc126 (5A) and Anc127 (5B) variants in terms of murine liver targeting. Each Anc126 and Anc127 variant was ranked from top to bottom based on liver uptake following IV administration of a multiplex Anc126 and Anc127 libraries. Columns show the positions of variation that make up the diversity within Anc126 and Anc127 libraries. Each position of variation has two possible residues, represented here as black or white. The percent of variants with that particular residue (i.e., black) in either the top or bottom half rank-order is indicated in the accompanying table.

FIGS. 6A-6B are fingerprint plots of Anc80 libraries having a glycine (black) or an alanine (white) at residue 266 administered to two different non-human primates.

FIG. 7 represents an alignment of VP1 capsid proteins that can be used in the methods described herein (AAV2 (SEQ ID NO:1); AAV1 (SEQ ID NO:4); AAV6 (SEQ ID NO:5); AAV3 (SEQ ID NO:6); AAV LK03 (SEQ ID NO:7); AAV7 (SEQ ID NO:8); AAV8 (SEQ ID NO:9); AAV hu.37 (SEQ ID NO:10); AAV rh.10 (SEQ ID NO:11); AAV9 (SEQ ID NO:12); AAV hu.68 (SEQ ID NO:13); AAV10 (SEQ ID NO:14); and AAV5 (SEQ ID NO:15)). The locations of the variable toggle residues described herein are boxed.

FIG. 8 represents an alignment of the amino acid sequences of AAV VP1 capsid proteins that can be used in the methods described herein (AAV5 (SEQ ID NO:15); AAV3-3 (SEQ ID NO:16); AAV4-4 (SEQ ID NO:17); AAV1-A (SEQ ID NO:18); hu. 46-A (SEQ ID NO:19); hu. 48-A (SEQ ID NO:20); hu. 44-A (SEQ ID NO:21); hu. 43-A (SEQ ID NO:22), AAV6-A (SEQ ID NO:23); hu. 34-B (SEQ ID NO:24), hu. 47-B (SEQ ID NO:25), hu. 29-B (SEQ ID NO:26), rh. 63-B (SEQ ID NO:27), hu. 56-B (SEQ ID NO:28), hu. 45-B (SEQ ID NO:29), rh. 57-B (SEQ ID NO:30), rh. 35-B (SEQ ID NO:31); rh. 58-B (SEQ ID NO:32); rh. 28-B (SEQ ID NO:33); rh. 51-B (SEQ ID NO:34); rh. 19-B (SEQ ID NO:35); rh. 49-B (SEQ ID NO:36), rh. 52-B (SEQ ID NO:37); rh. 13-B (SEQ ID NO:38); AAV2-B (SEQ ID NO:39); rh. 20-B (SEQ ID NO:40); rh. 24-B (SEQ ID NO:41); rh. 64-B (SEQ ID NO:42); hu. 27-B (SEQ ID NO:43); hu. 21-B (SEQ ID NO:44); hu. 22-B (SEQ ID NO:45); hu. 23-B (SEQ ID NO:46); hu. 7-C(SEQ ID NO:47); hu. 61-C(SEQ ID NO:48); rh. 56-C(SEQ ID NO:49); hu. 9-C(SEQ ID NO:50); hu. 54-C(SEQ ID NO:51); hu. 53-C(SEQ ID NO:52); hu. 60-C (SEQ ID NO:53); hu. 55-C(SEQ ID NO:54); hu. 2-C(SEQ ID NO:55); hu. 1-C(SEQ ID NO:56); hu. 18-C(SEQ ID NO:57); hu. 3-C(SEQ ID NO:58); hu. 25-C(SEQ ID NO:59); hu. 15-C(SEQ ID NO:60); hu. 16-C(SEQ ID NO:61); hu. 11-C(SEQ ID NO:62); hu. 10-C(SEQ ID NO:63); hu. 4-C(SEQ ID NO:64); rh. 54-D (SEQ ID NO:65); rh. 48-D (SEQ ID NO:66); rh. 55-D (SEQ ID NO:67); rh. 62-D (SEQ ID NO:68); AAV7-D (SEQ ID NO:69); rh. 52-E (SEQ ID NO:70); rh. 51-E (SEQ ID NO:71); hu. 39-E (SEQ ID NO:72); rh. 53-E (SEQ ID NO:73); hu. 37-E (SEQ ID NO:74); rh. 43-E (SEQ ID NO:75); rh. 50-E (SEQ ID NO:76); rh. 49-E (SEQ ID NO:77); rh. 61-E (SEQ ID NO:78); hu. 41-E (SEQ ID NO:79); rh. 64-E (SEQ ID NO:80); hu. 42-E (SEQ ID NO:81); rh. 57-E (SEQ ID NO:82); rh. 40-E (SEQ ID NO:83); AAV8-E (SEQ ID NO:153); rh.58-E (SEQ ID NO:2); rh.40-E (SEQ ID NO:3); hu. 67-E (SEQ ID NO:84); hu. 17-E (SEQ ID NO:85); hu. 6-E (SEQ ID NO:86); hu. 66-E (SEQ ID NO:87); rh. 38-E (SEQ ID NO:88); hu. 32-F (SEQ ID NO:89); AAV9/hu (SEQ ID NO:90); and hu. 31-F (SEQ ID NO:91). The locations of the variable toggle residues described herein are boxed.

FIG. 9 represents an alignment of the amino acid sequences of AAV Anc capsid proteins that can be used in the methods described herein (AAV2 (SEQ ID NO:1); Anc80 (SEQ ID NO:92), Anc81 (SEQ ID NO:93), Anc82 (SEQ ID NO:94), Anc83 (SEQ ID NO:95), Anc84 (SEQ ID NO:96), Anc94 (SEQ ID NO:97), Anc113 (SEQ ID NO:98), Anc126 (SEQ ID NO:99), Anc127 (SEQ ID NO:100), Anc80L27 (SEQ ID NO:101), Anc80L59 (SEQ ID NO:102), Anc80L60 (SEQ ID NO:103), Anc80L62 (SEQ ID NO:104), Anc80L65 (SEQ ID NO:105), Anc80L33 (SEQ ID NO:106), Anc80L36 (SEQ ID NO:107), Anc80L44 (SEQ ID NO:108); Anc80L1 (SEQ ID NO:109); Anc110 (SEQ ID NO:110), Anc80DI (SEQ ID NO:111).

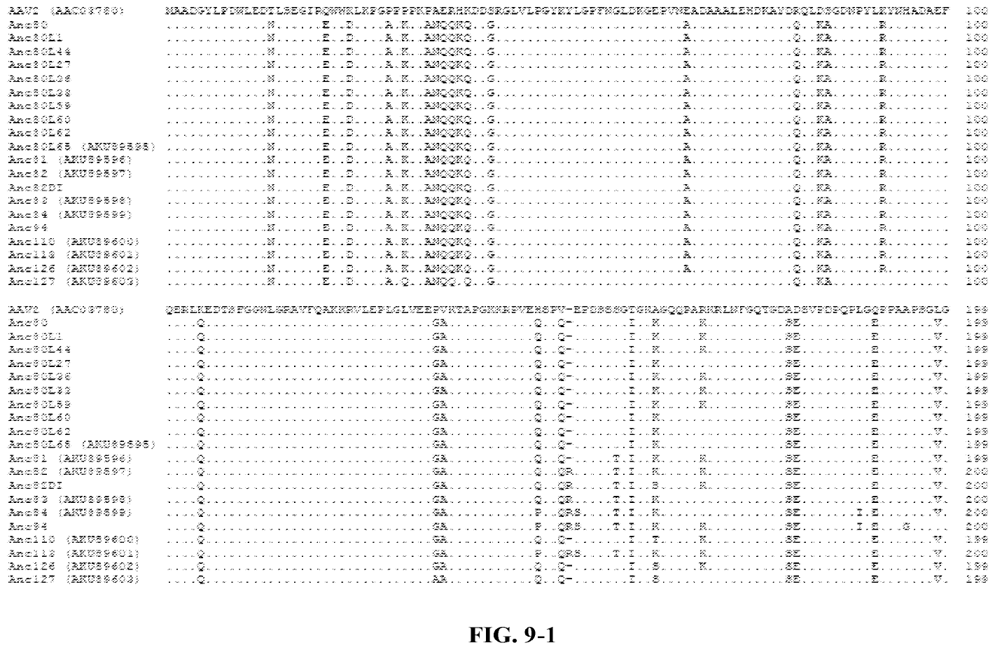

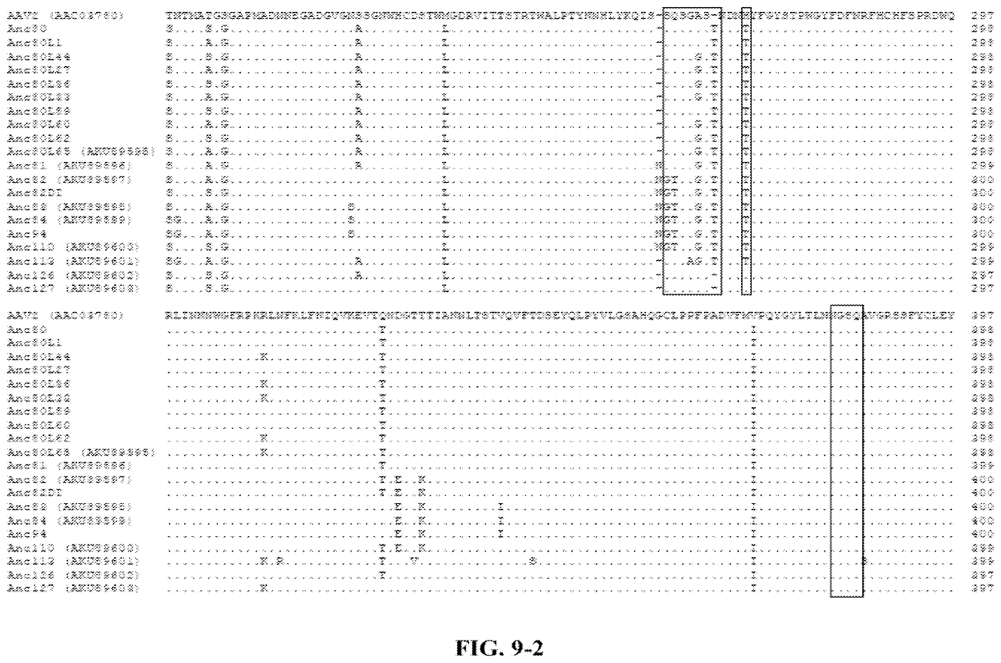

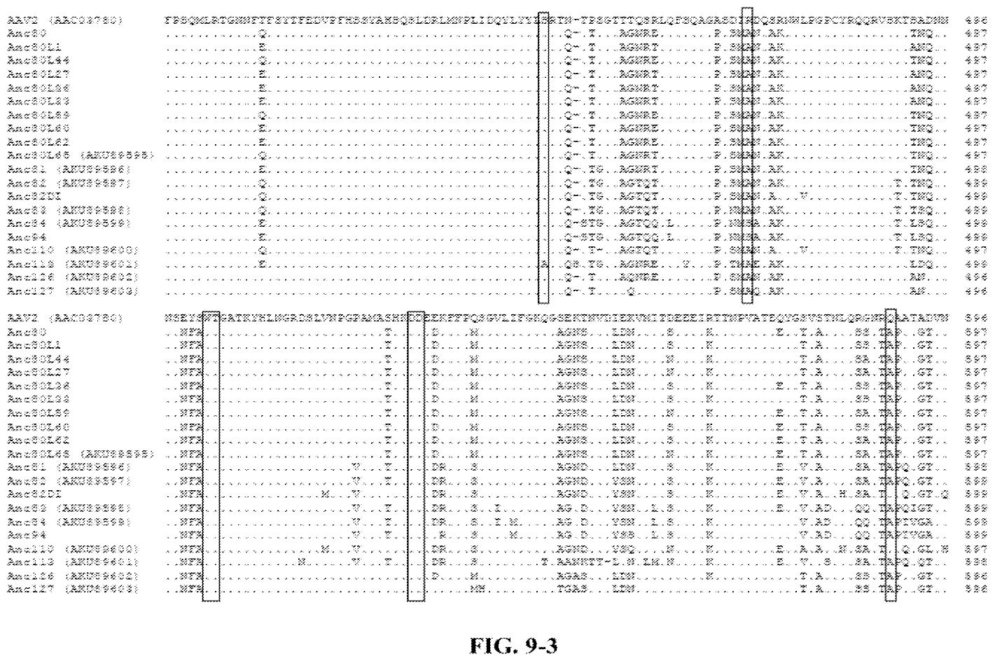

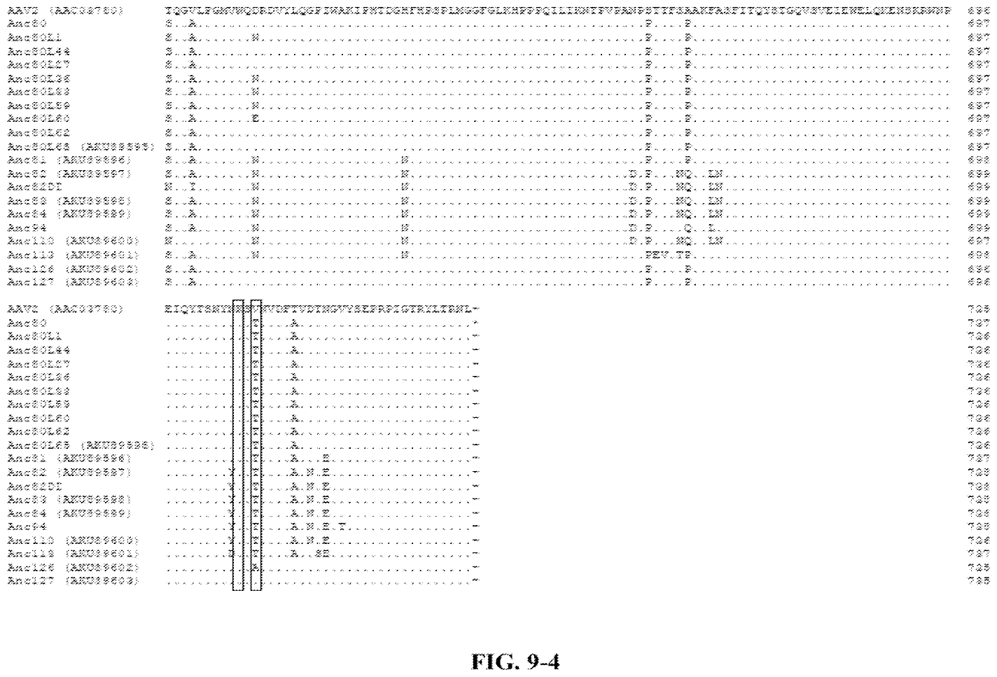

Among the sequences, SEQ ID NOs: 92-100 are sequences of the ancestral AAV capsid libraries disclosed in U.S. Pat. No. 9,695,220, incorporated by reference in its entirety herein. The libraries include Anc80 (SEQ ID NO:92), Anc81 (SEQ ID NO:93), Anc82 (SEQ ID NO:94), Anc83 (SEQ ID NO:95), Anc84 (SEQ ID NO:96), Anc94 (SEQ ID NO:97), Anc113 (SEQ ID NO:98), Anc126 (SEQ ID NO:99), and Anc127 (SEQ ID NO:100). FIG. 9 was generated using single member sequences (SEQ ID NOs 92-100) of each library, but the same analysis and alignment can be done with any other member of the libraries to identify locations of the variable toggle residues.

The locations of the variable toggle residues described herein are boxed. One or more amino acids in the toggle sites can be substituted, inserted and/or deleted as described herein to achieve a desired AAV biodistribution.

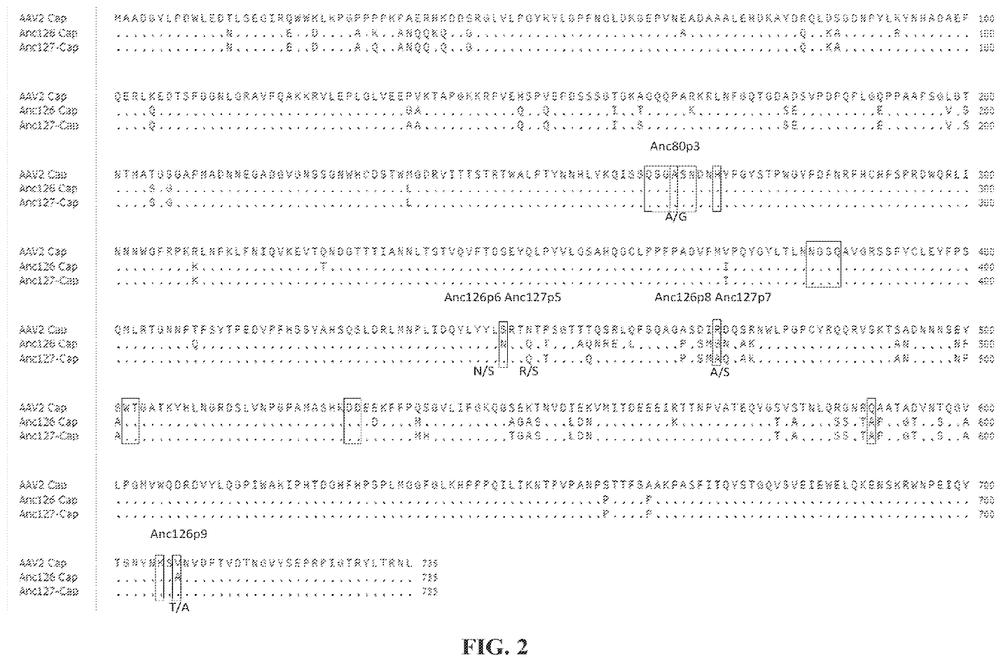

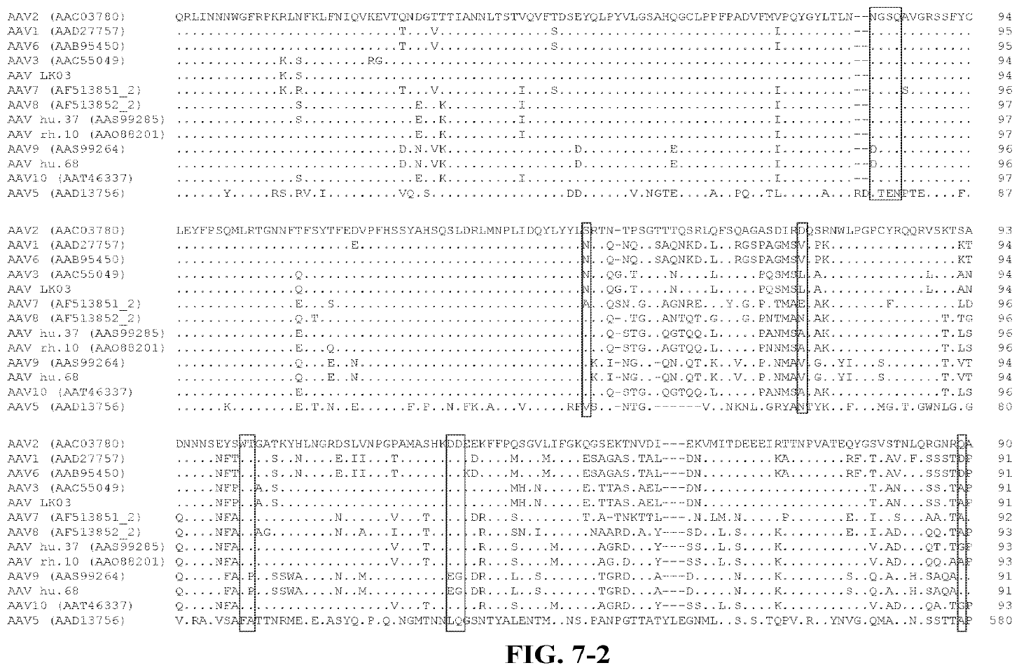

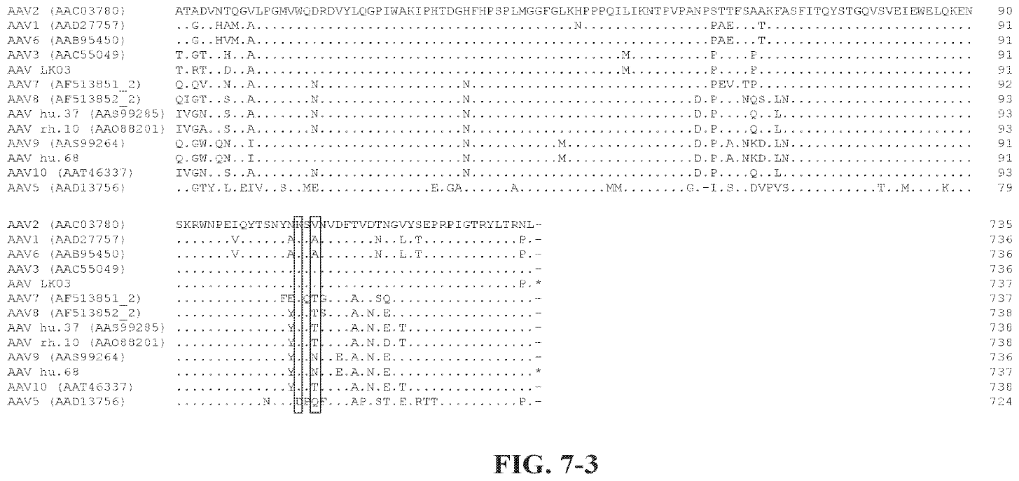

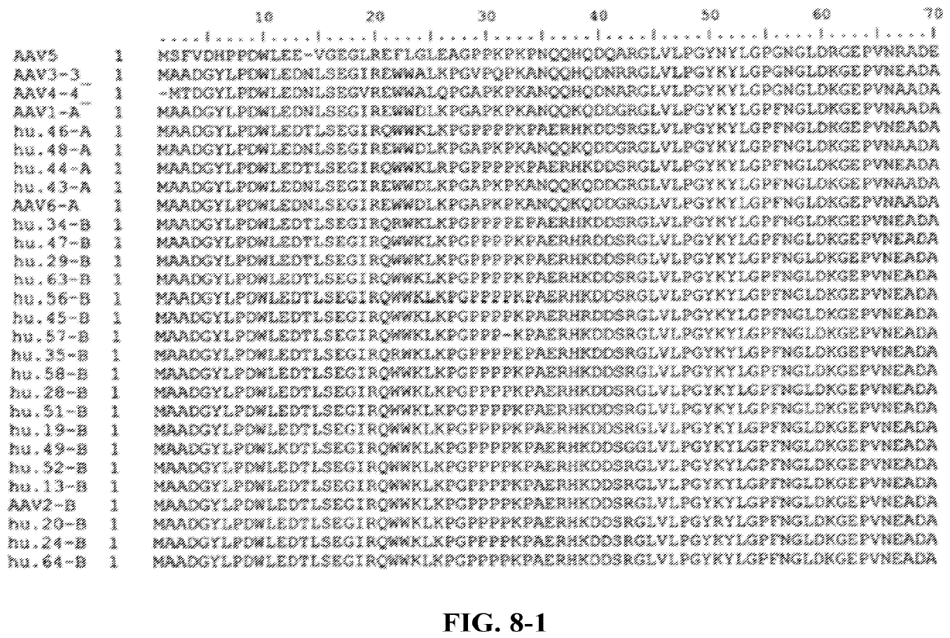

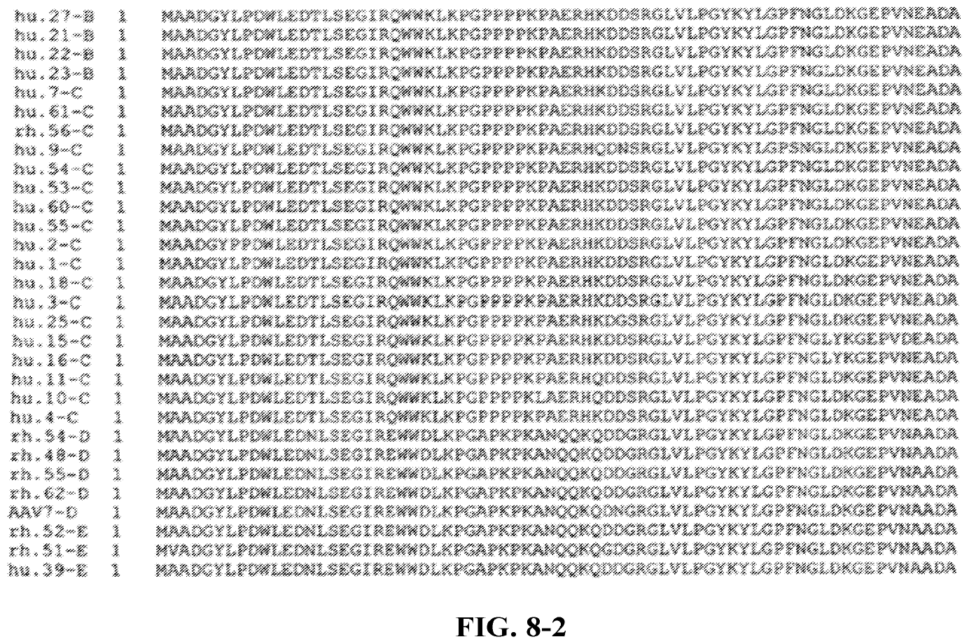

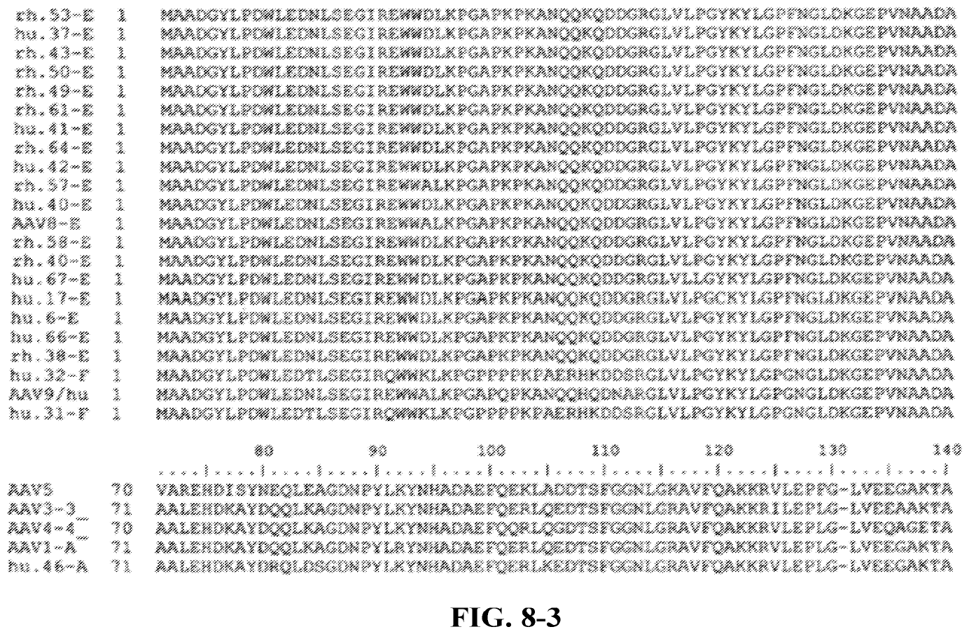

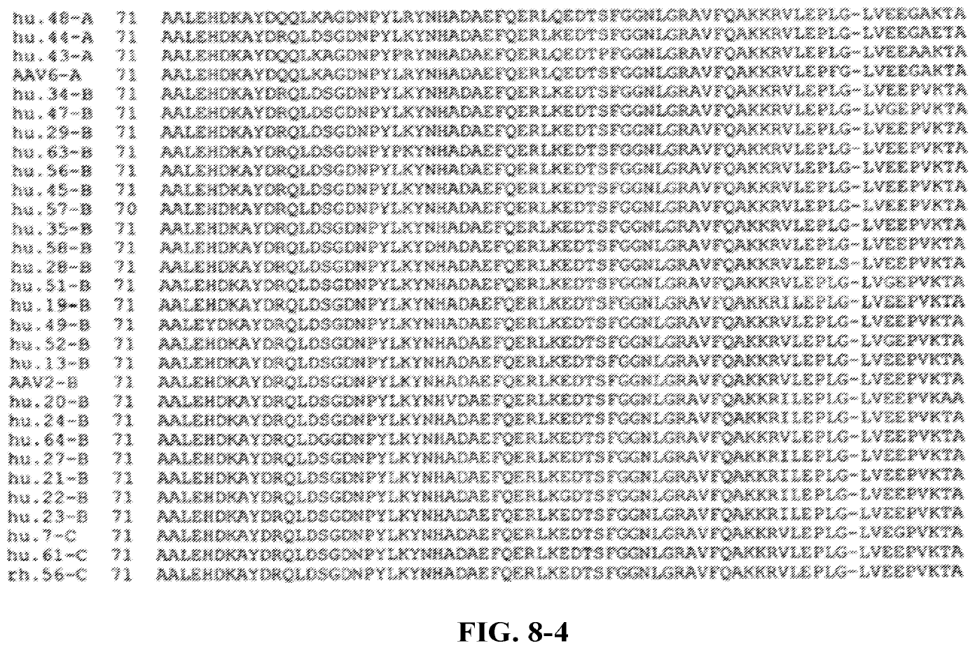

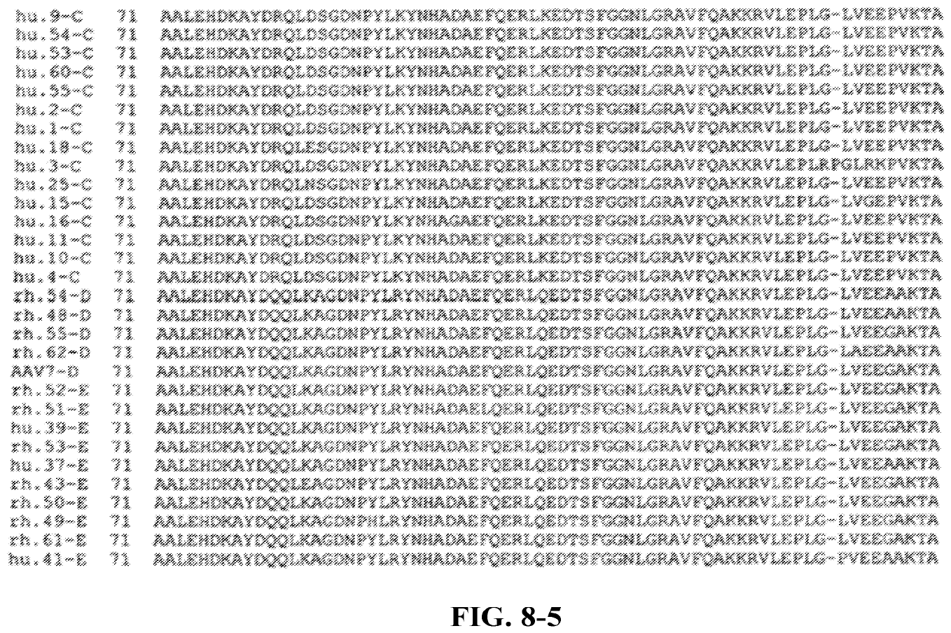

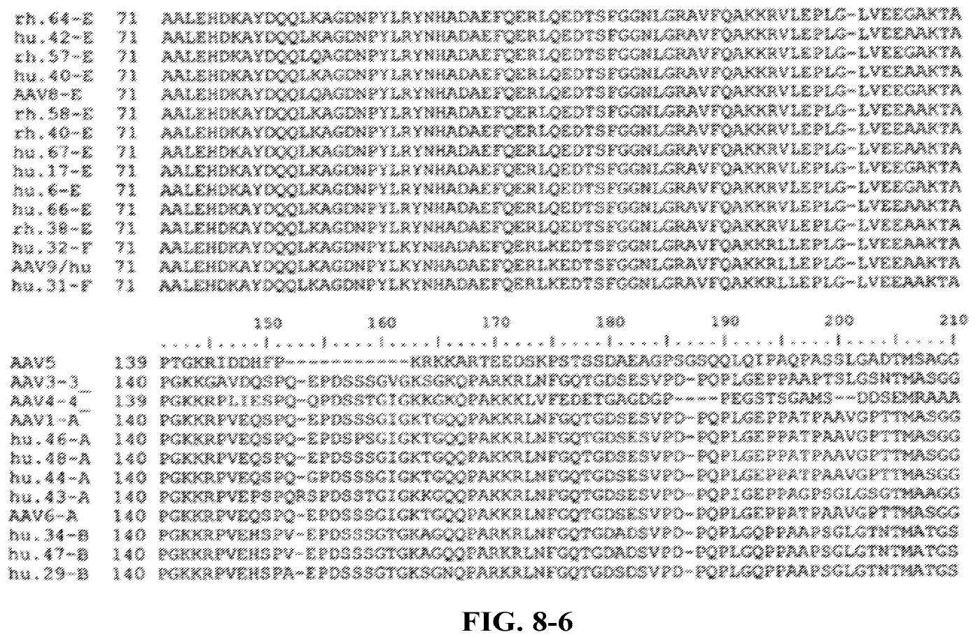

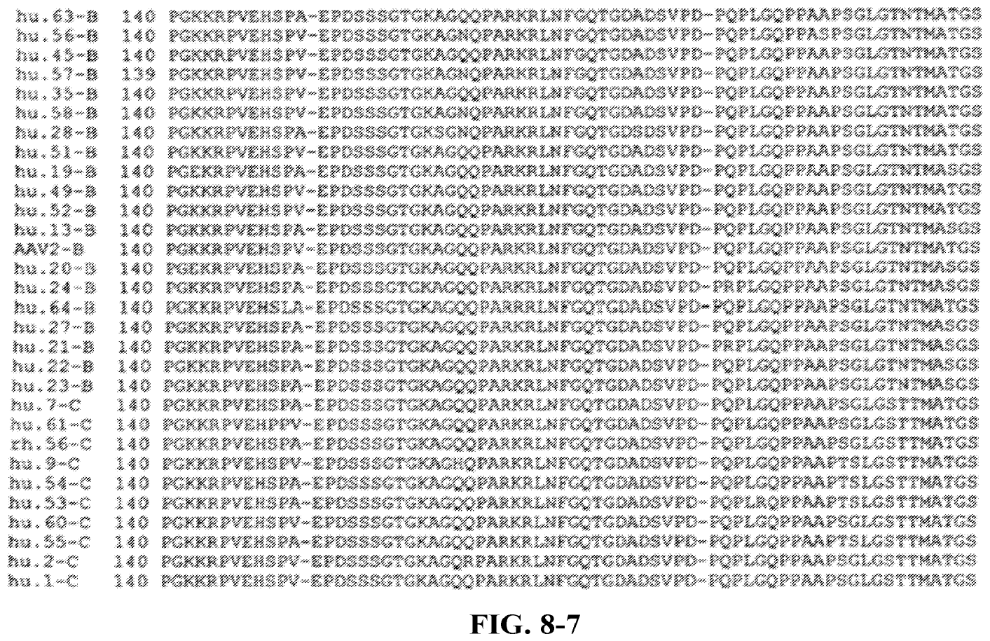

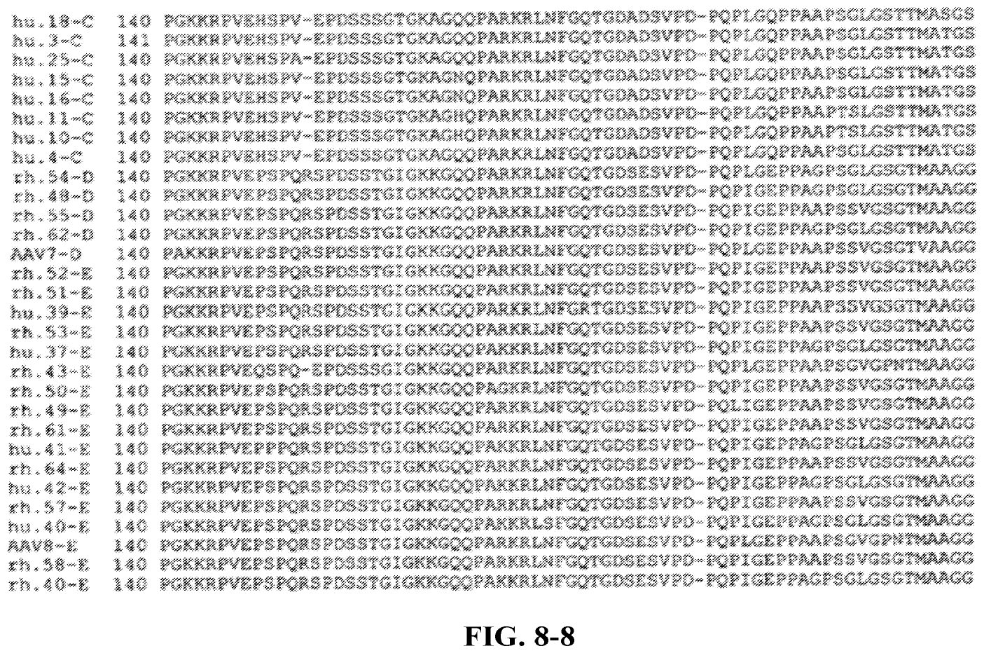

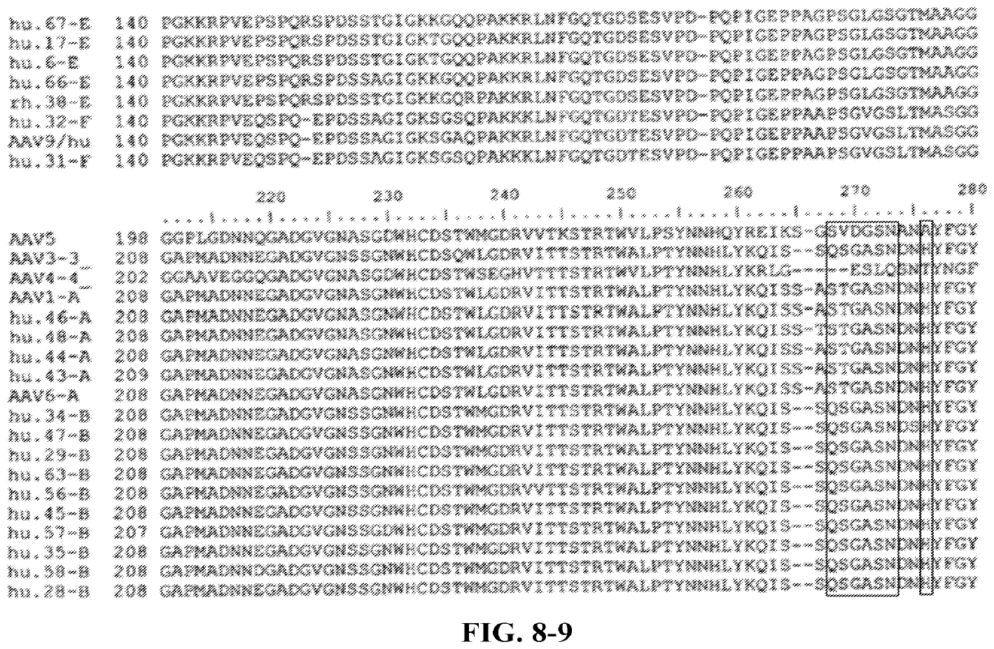

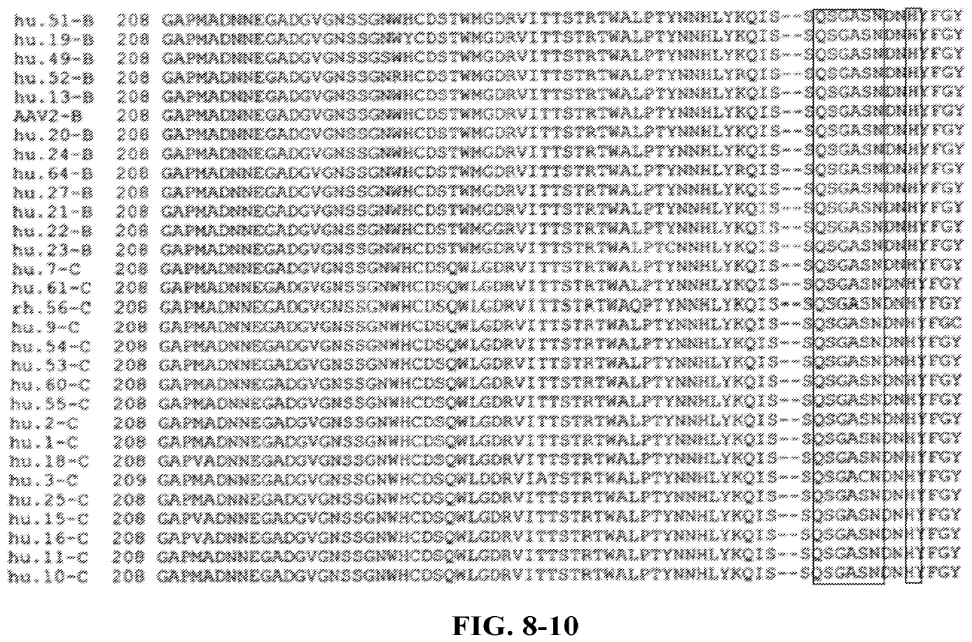

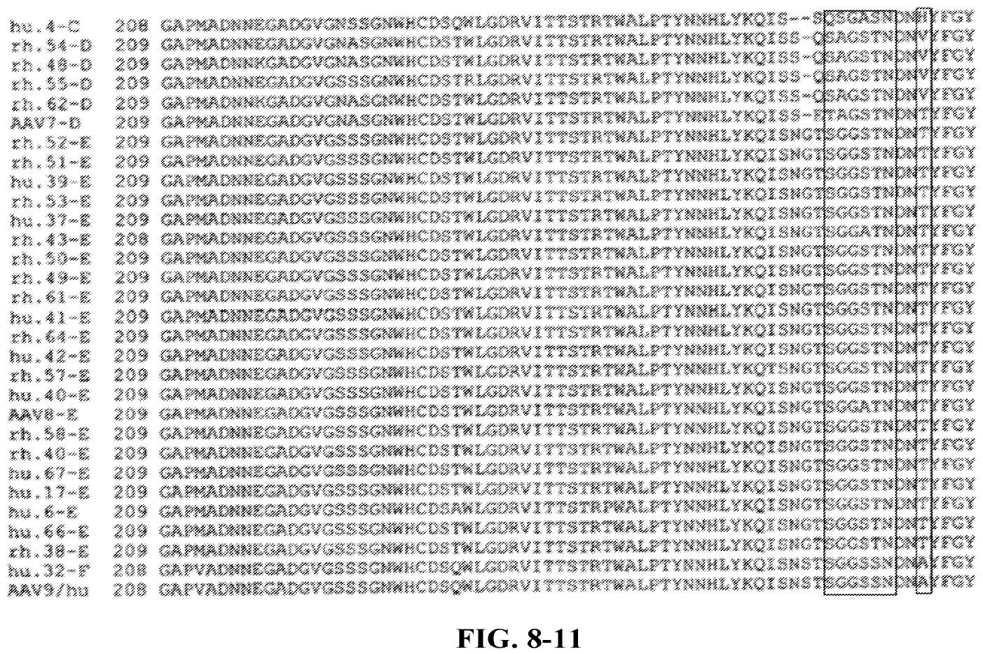

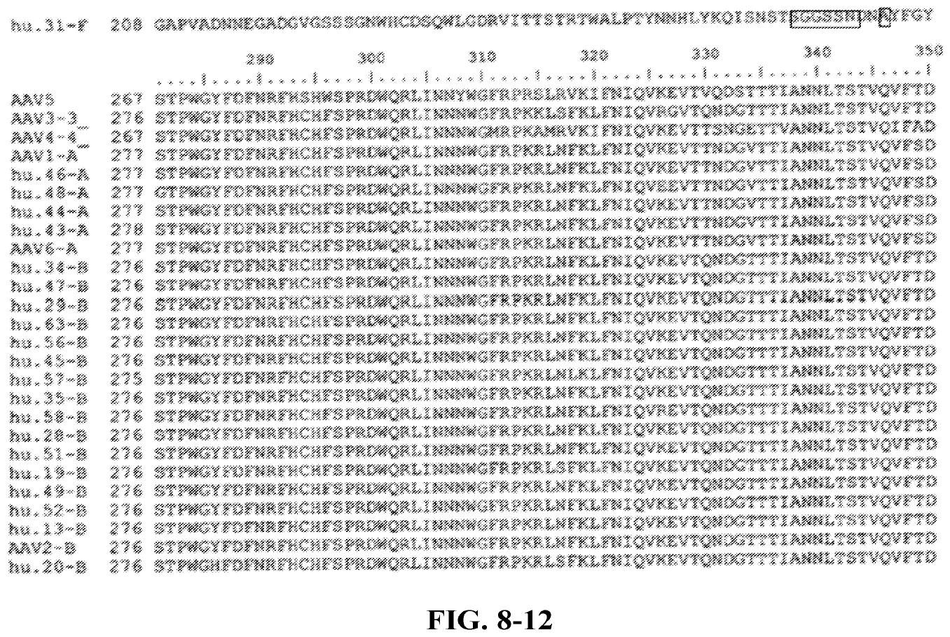

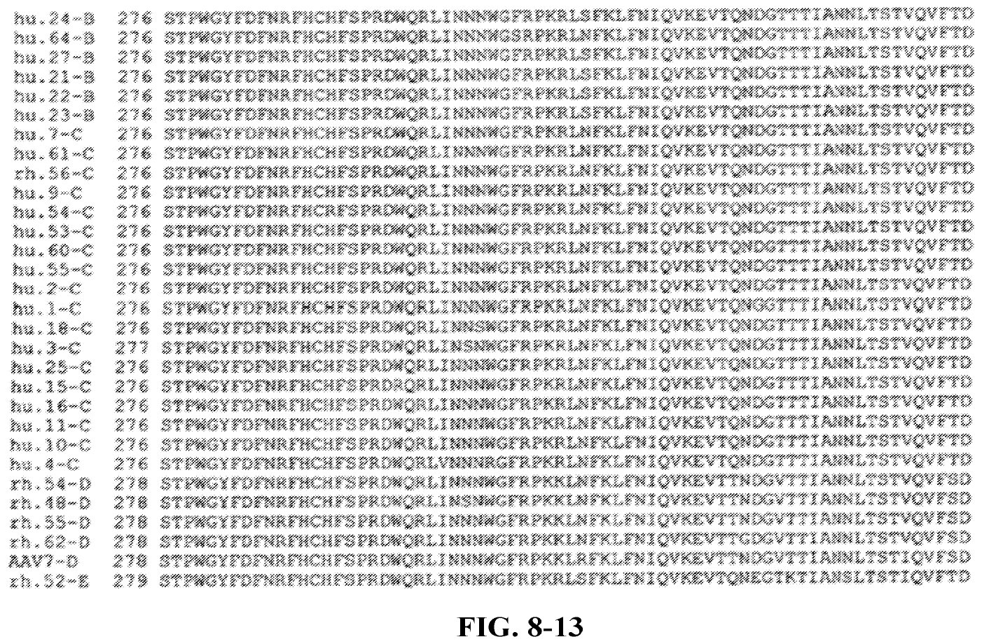

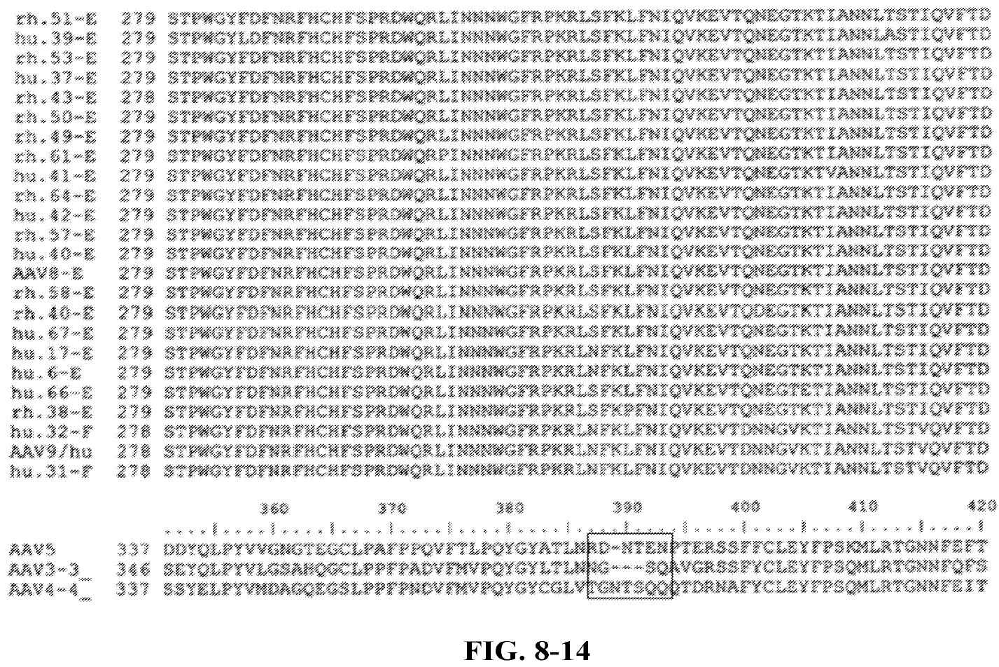

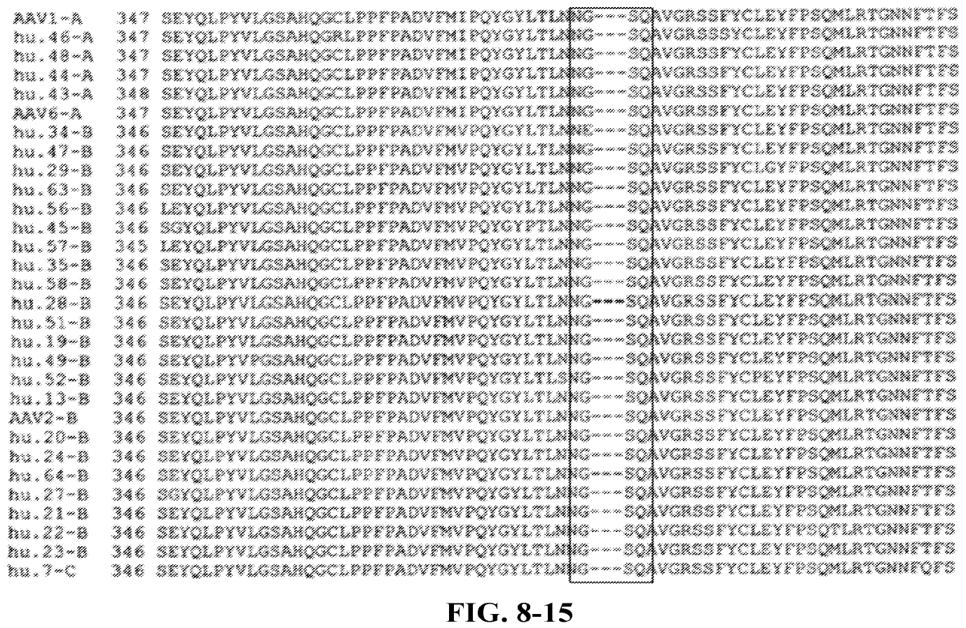

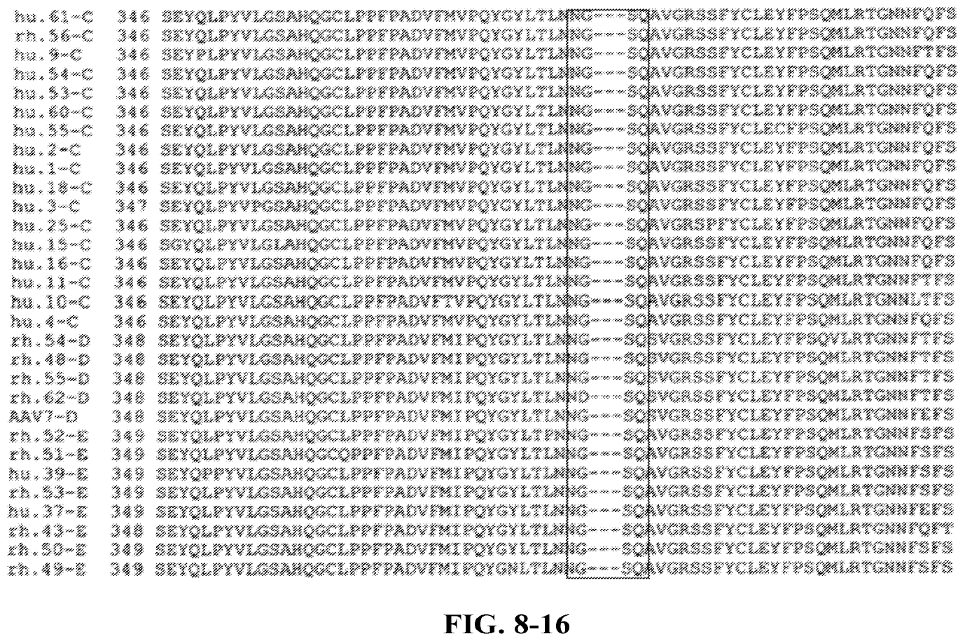

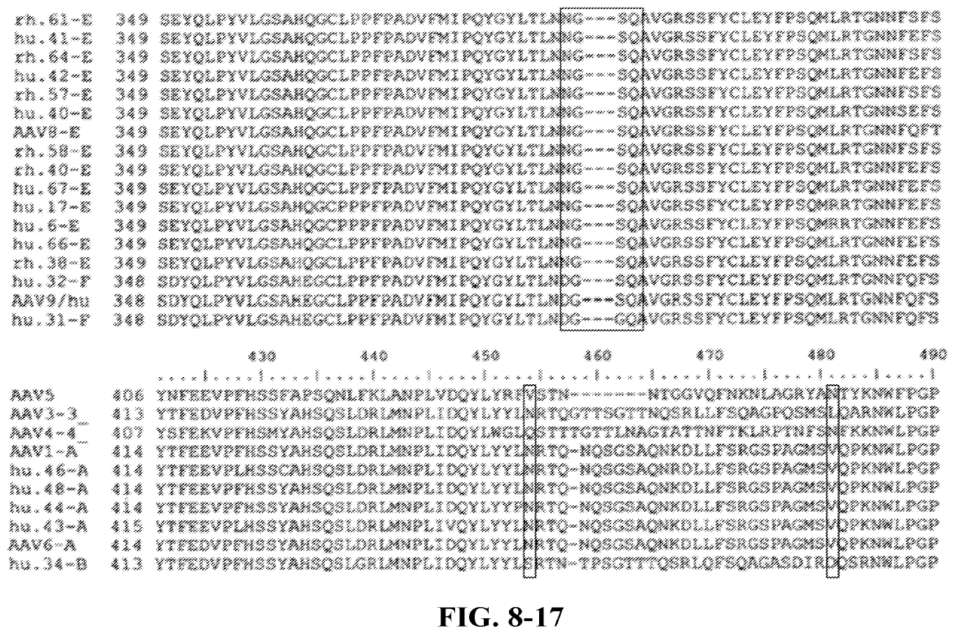

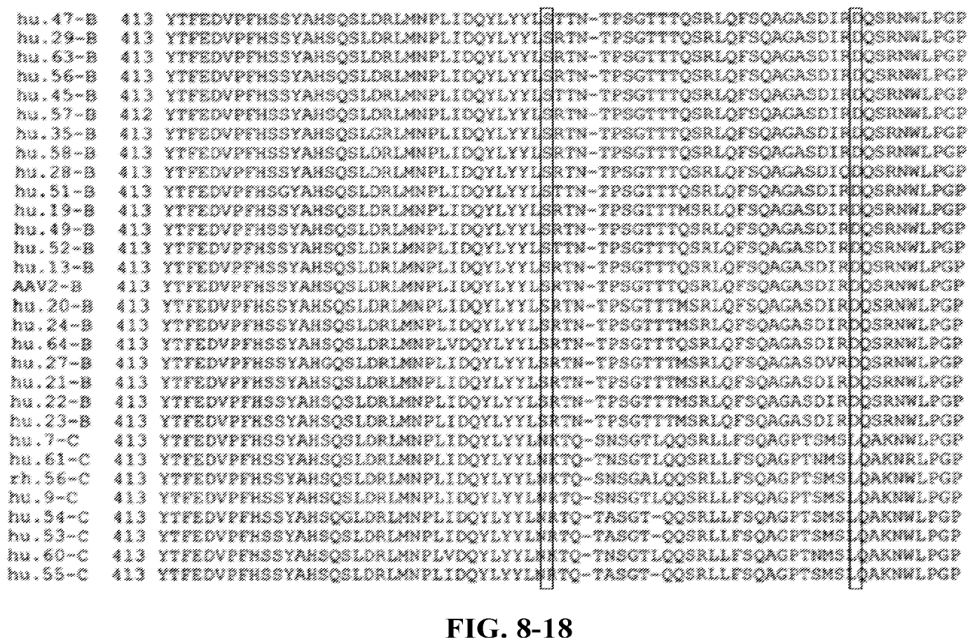

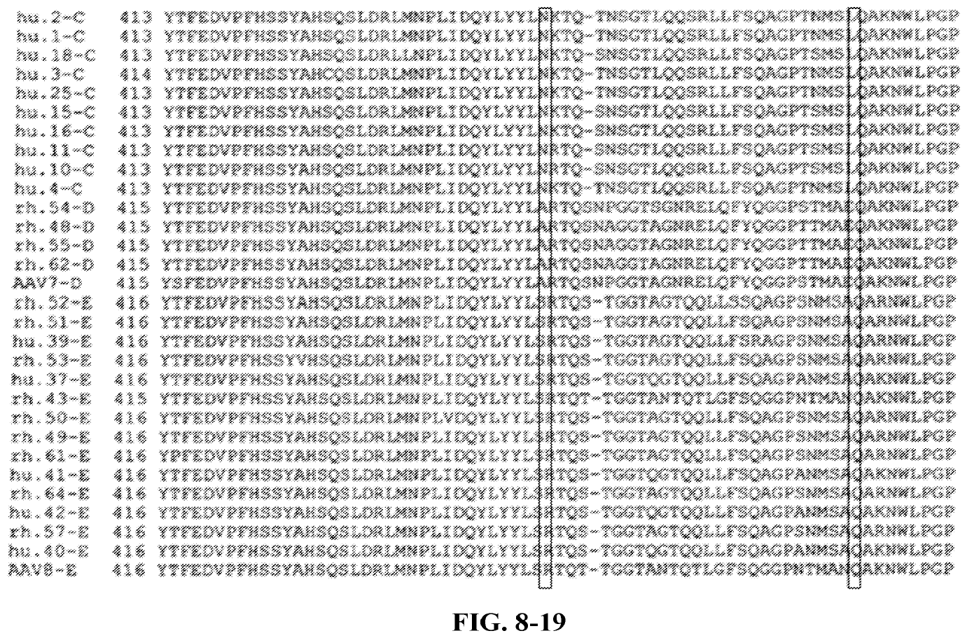

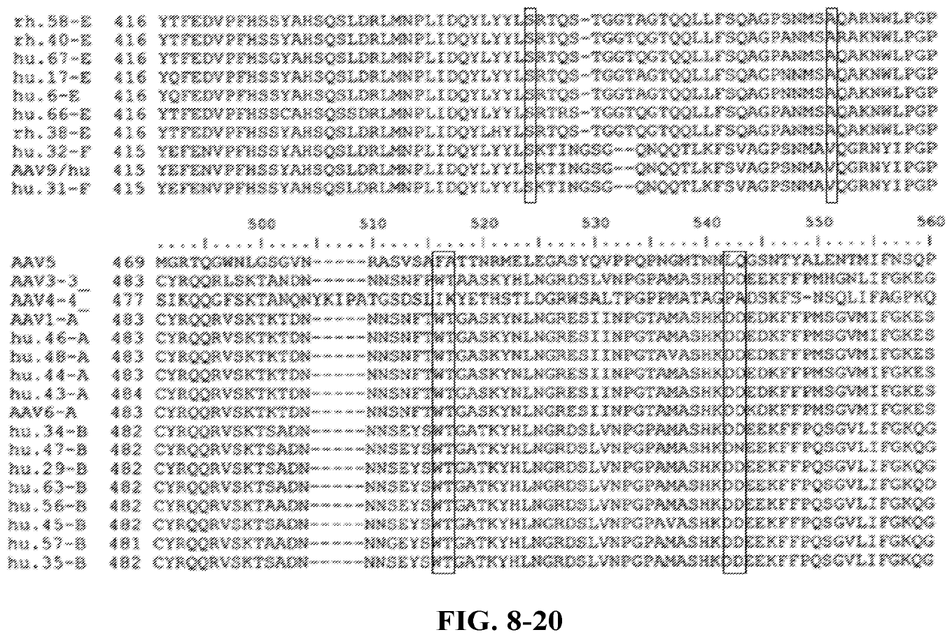

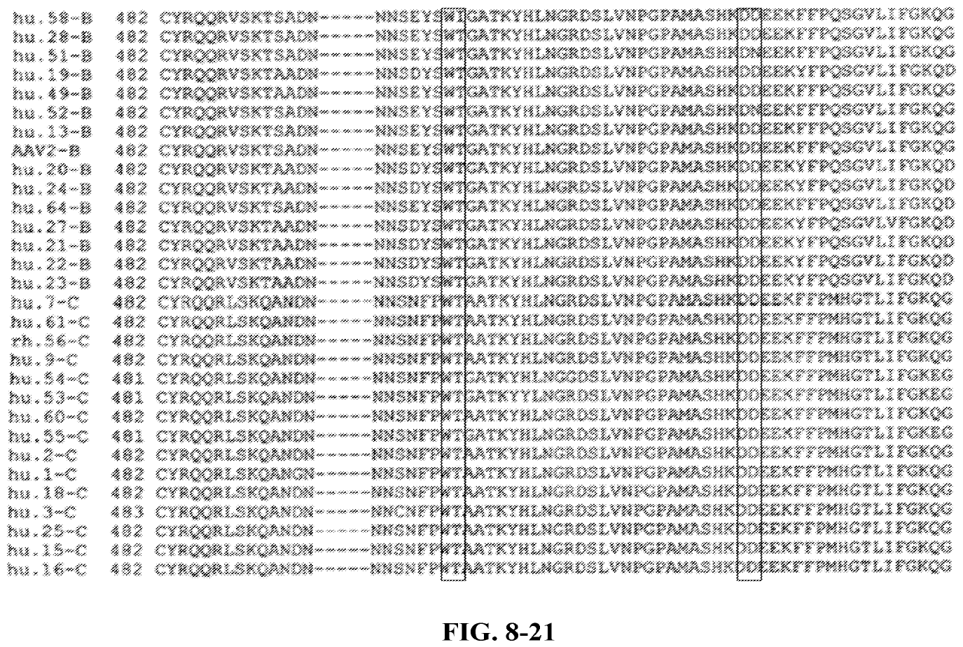

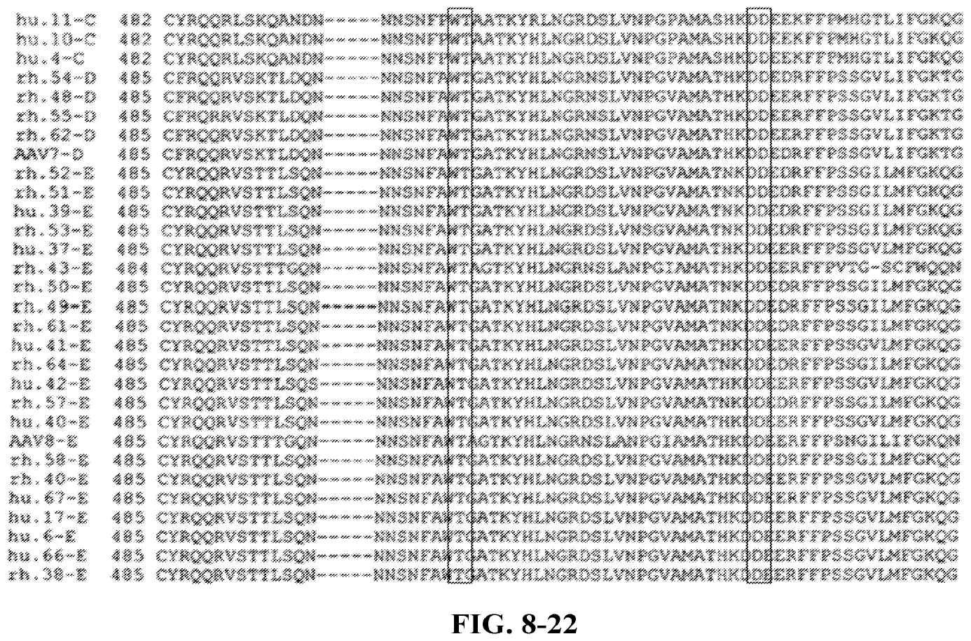

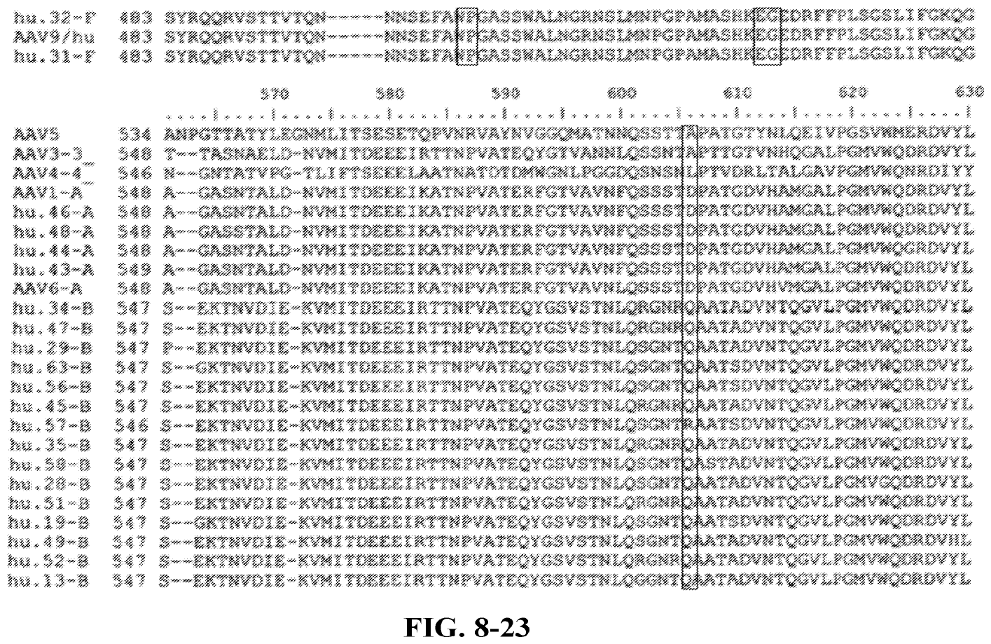

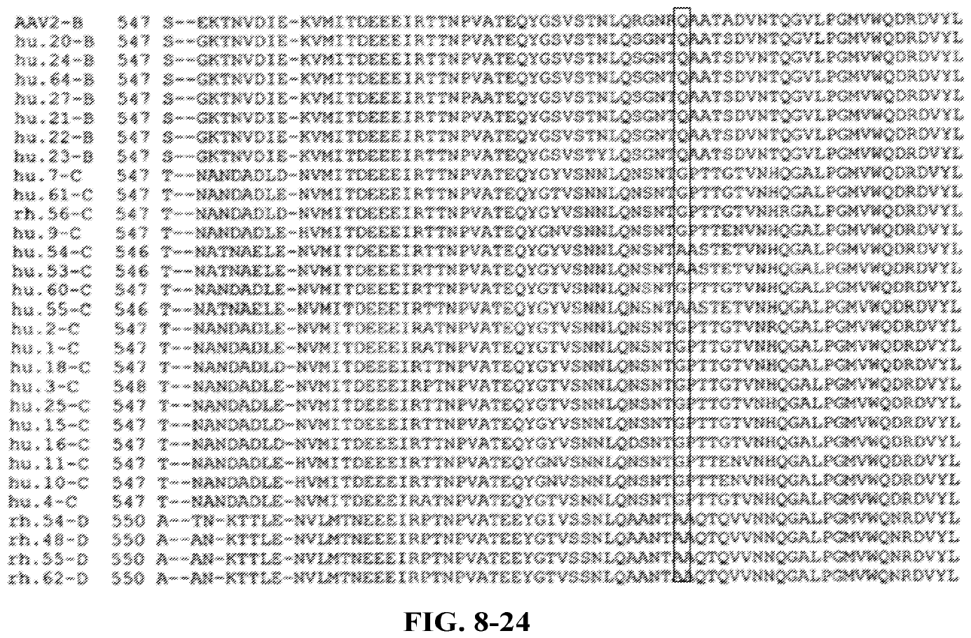

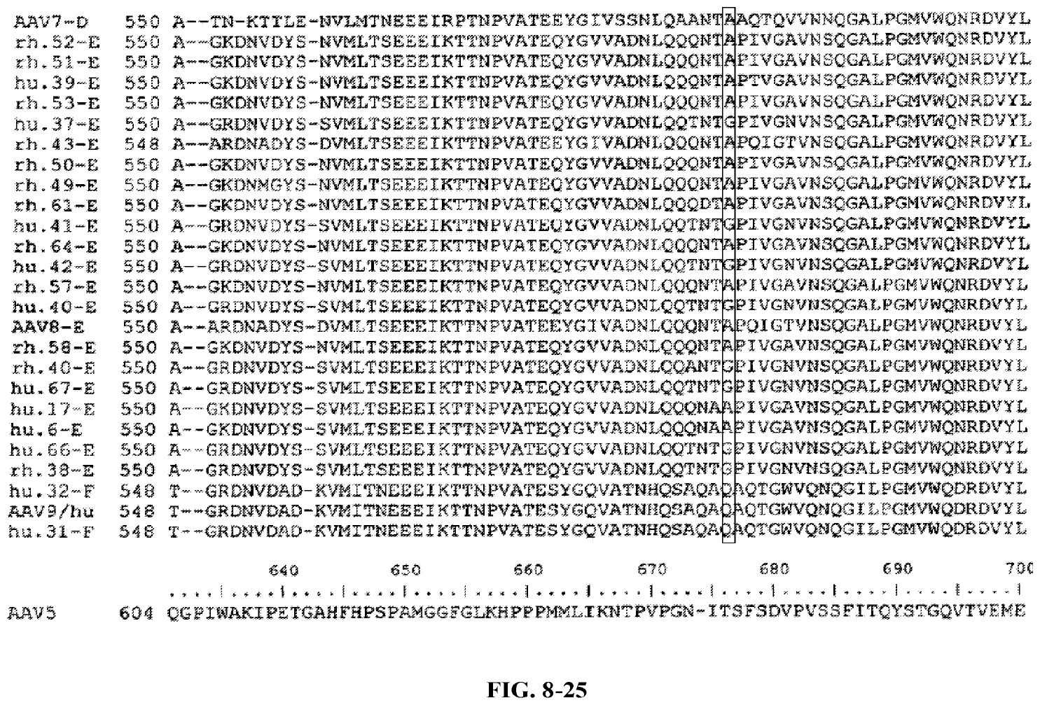

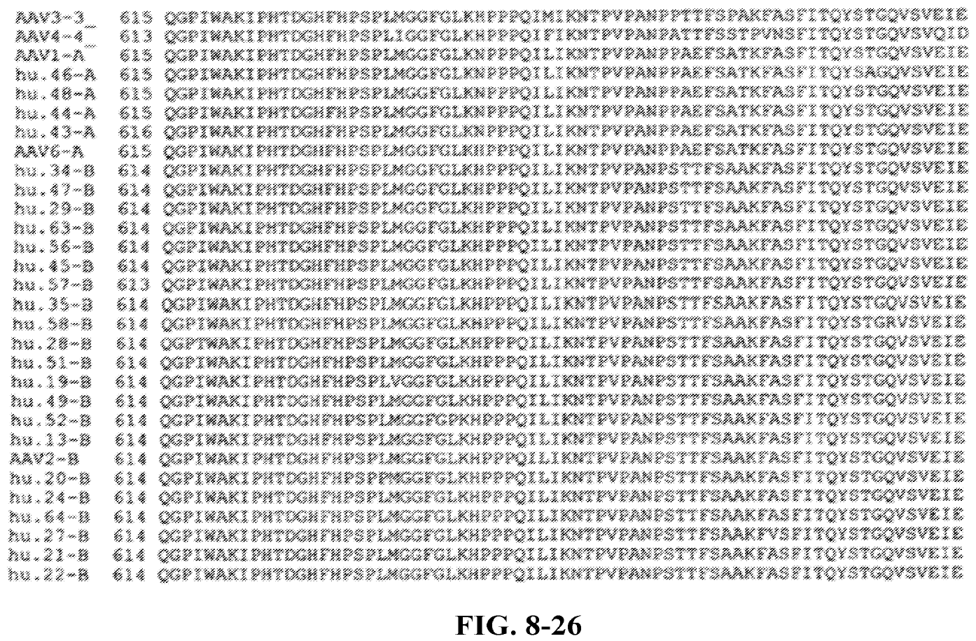

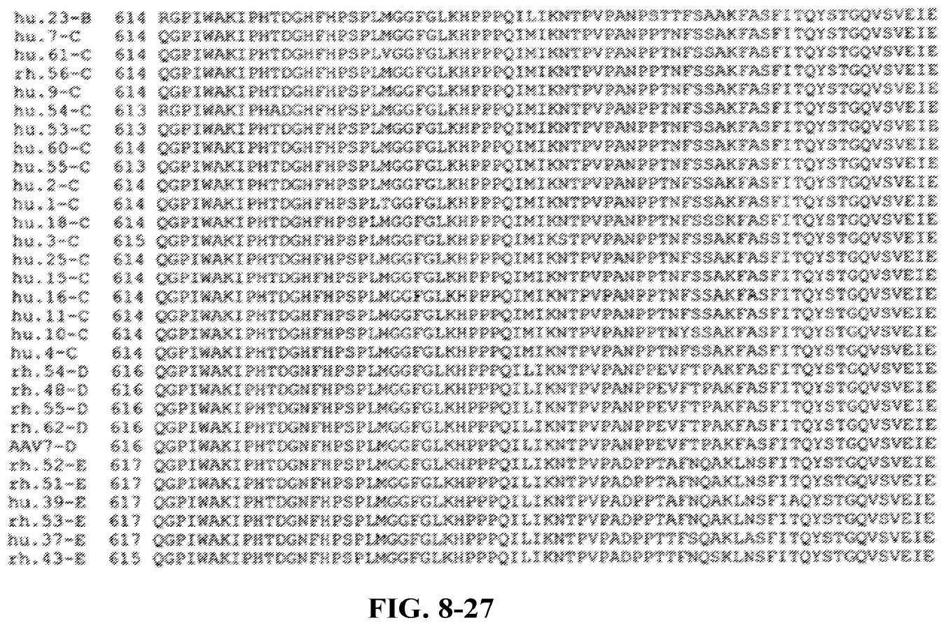

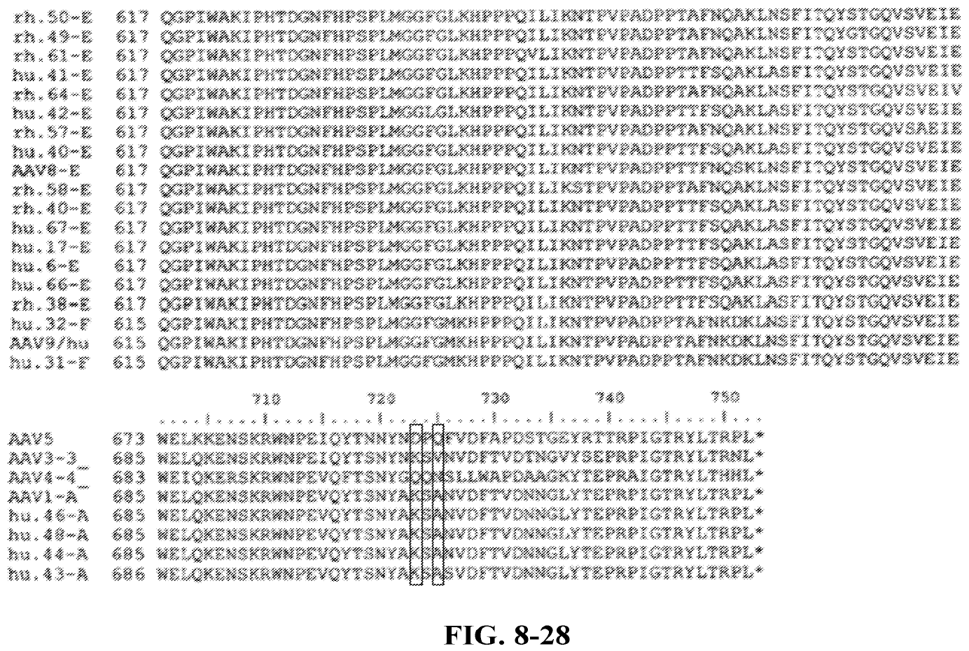

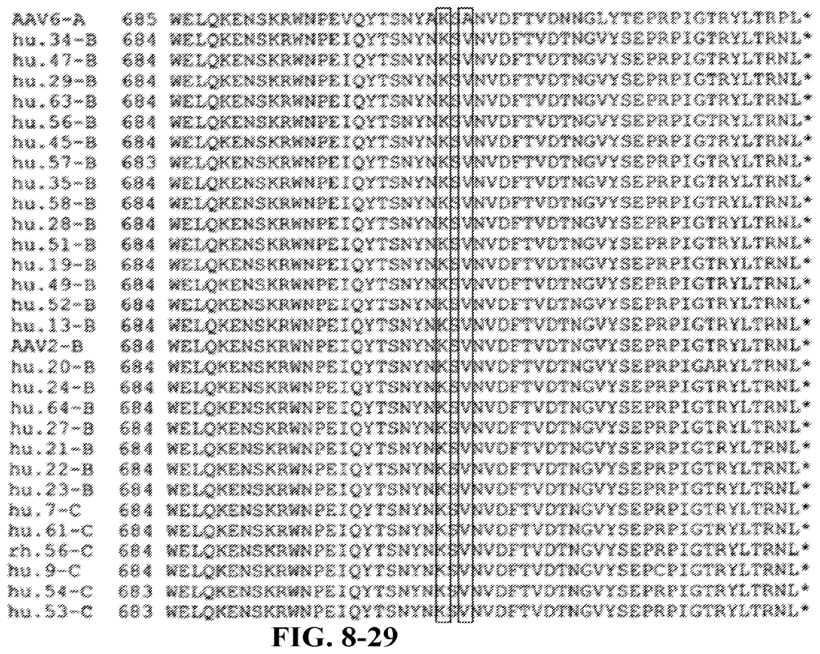

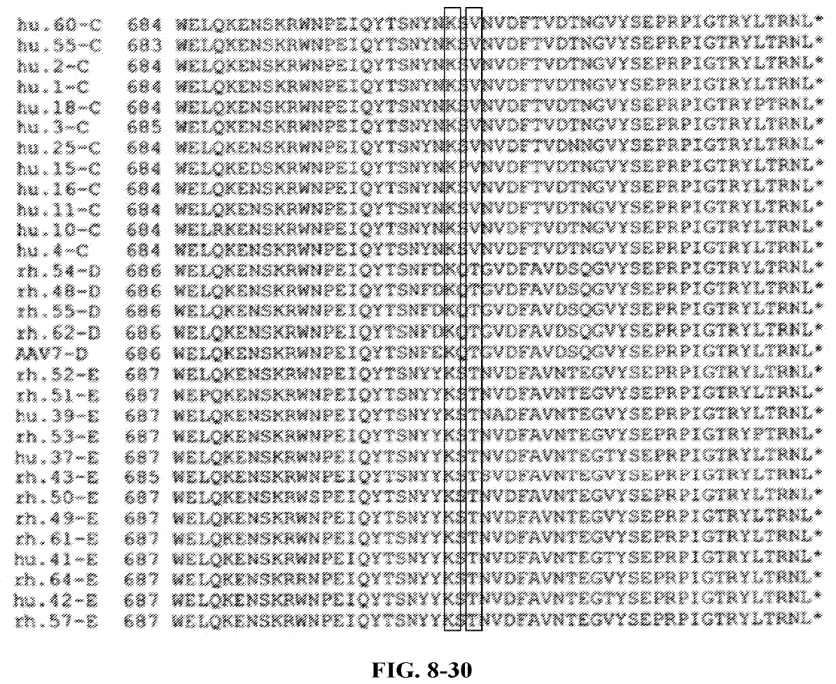

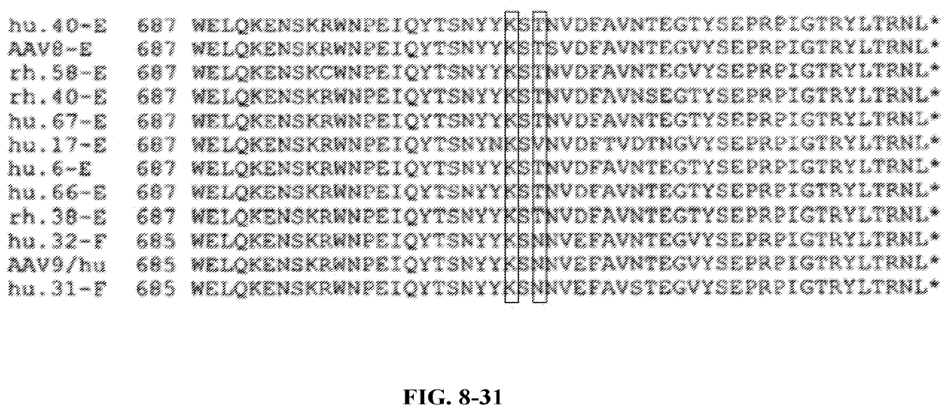

### DESCRIPTION AND LOCATION OF SEQUENCES

| SEQ ID NO: | VP1 capsid serotype: | Location: |
|---|---|---|
| 1 | AAV2 | FIG. 2, 7 |
| 2 | rh.58-E | FIG. 8 |
| 3 | rh.40-E | FIG. 8 |
| 4 | AAV1 | FIG. 7 |
| 5 | AAV6 | FIG. 7 |
| 6 | AAV3 | FIG. 7 |
| 7 | AAV LK03 | FIG. 7 |
| 8 | AAV7 | FIG. 7 |
| 9 | AAV8 | FIG. 7 |
| 10 | hu.37 | FIG. 7 |
| 11 | rh.10 | FIG. 7 |
| 12 | AAV9 | FIG. 7 |
| 13 | hu.68 | FIG. 7 |
| 14 | AAV10 | FIG. 7 |
| 15 | AAV5 | FIG. 7, 8 |
| 16 | AAV3-3 | FIG. 8 |
| 17 | AAV4-4 | FIG. 8 |
| 18 | AAV1-A | FIG. 8 |
| 19 | hu.46-A | FIG. 8 |
| 20 | hu.48-A | FIG. 8 |
| 21 | hu.44-A | FIG. 8 |
| 22 | hu.43-A | FIG. 8 |
| 23 | AAV6-A | FIG. 8 |
| 24 | hu.34-B | FIG. 8 |
| 25 | hu.47-B | FIG. 8 |
| 26 | hu.29-B | FIG. 8 |
| 27 | rh.63-B | FIG. 8 |
| 28 | hu.56-B | FIG. 8 |
| 29 | hu.45-B | FIG. 8 |
| 30 | rh.57-B | FIG. 8 |
| 31 | rh.35-B | FIG. 8 |
| 32 | rh.58-B | FIG. 8 |
| 33 | rh.28-B | FIG. 8 |
| 34 | rh.51-B | FIG. 8 |
| 35 | rh.19-B | FIG. 8 |
| 36 | rh.49-B | FIG. 8 |
| 37 | rh.52-B | FIG. 8 |
| 38 | rh.13-B | FIG. 8 |
| 39 | AAV2-B | FIG. 8 |
| 40 | rh.20-B | FIG. 8 |
| 41 | rh.24-B | FIG. 8 |
| 42 | rh.64-B | FIG. 8 |
| 43 | hu.27-B | FIG. 8 |
| 44 | hu.21-B | FIG. 8 |
| 45 | hu.22-B | FIG. 8 |
| 46 | hu.23-B | FIG. 8 |
| 47 | hu.7-C | FIG. 8 |
| 48 | hu.61-C | FIG. 8 |
| 49 | rh.56-C | FIG. 8 |
| 50 | hu.9-C | FIG. 8 |
| 51 | hu.54-C | FIG. 8 |
| 52 | hu.53-C | FIG. 8 |
| 53 | hu.60-C | FIG. 8 |
| 54 | hu.55-C | FIG. 8 |
| 55 | hu.2-C | FIG. 8 |
| 56 | hu.1-C | FIG. 8 |
| 57 | hu.18-C | FIG. 8 |
| 58 | hu.3-C | FIG. 8 |
| 59 | hu.25.C | FIG. 8 |
| 60 | hu.15-C | FIG. 8 |
| 61 | hu.16-C | FIG. 8 |
| 62 | hu.11-C | FIG. 8 |
| 63 | hu.10-C | FIG. 8 |
| 64 | hu.4-C | FIG. 8 |
| 65 | rh.54-D | FIG. 8 |
| 66 | rh.48-D | FIG. 8 |
| 67 | rh.55-D | FIG. 8 |
| 68 | rh.62-D | FIG. 8 |
| 69 | AAV7-D | FIG. 8 |
| 70 | rh.52-E | FIG. 8 |
| 71 | rh.51-E | FIG. 8 |
| 72 | hu.39-E | FIG. 8 |
| 73 | rh.53-E | FIG. 8 |
| 74 | hu.37-E | FIG. 8 |
| 75 | rh.43-E | FIG. 8 |
| 76 | rh.50-E | FIG. 8 |
| 77 | rh.49-E | FIG. 8 |
| 78 | rh.61-E | FIG. 8 |
| 79 | hu.41-E | FIG. 8 |
| 80 | rh.64-E | FIG. 8 |
| 81 | hu.42-E | FIG. 8 |
| 82 | rh.57-E | FIG. 8 |
| 83 | rh.40-E | FIG. 8 |
| 84 | hu.67-E | FIG. 8 |
| 85 | hu.17-E | FIG. 8 |
| 86 | hu.6-E | FIG. 8 |
| 87 | hu.66-E | FIG. 8 |
| 88 | rh.38-E | FIG. 8 |
| 89 | hu.32-F | FIG. 8 |
| 90 | AAV9/hu | FIG. 8 |
| 91 | hu.31-F | FIG. 8 |
| 92 | Anc80 | FIG. 9 |
| 93 | Anc81 | FIG. 9 |
| 94 | Anc82 | FIG. 9 |
| 95 | Anc83 | FIG. 9 |
| 96 | Anc84 | FIG. 9 |
| 97 | Anc94 | FIG. 9 |
| 98 | Anc113 | FIG. 9 |
| 99 | Anc126 | FIG. 2, 9 |
| 100 | Anc127 | FIG. 2, 9 |
| 101 | Anc80L27 | FIG. 9 |
| 102 | Anc80L59 | FIG. 9 |
| 103 | Anc80L60 | FIG. 9 |
| 104 | Anc80L62 | FIG. 9 |
| 105 | Anc80L65 | FIG. 9 |
| 106 | Anc80L33 | FIG. 9 |
| 107 | Anc80L36 | FIG. 9 |
| 108 | Anc80L44 | FIG. 9 |
| 109 | Anc80L1 | FIG. 9 |
| 110 | Anc110 | FIG. 9 |
| 111 | Anc80DI | FIG. 9 |
| 112 | AAV2 and modifications thereof | Appendix |
| 113 | AAV1 and modifications thereof | Appendix |
| 114 | AAV6 and modifications thereof | Appendix |
| 115 | AAV3 and modifications thereof | Appendix |
| 116 | AAV LK03 and modifications thereof | Appendix |
| 117 | AAV7 and modifications thereof | Appendix |
| 118 | AAV8 and modifications thereof | Appendix |
| 119 | AAV hu.37 and modifications thereof | Appendix |
| 120 | AAV rh.10 and modifications thereof | Appendix |
| 121 | AA9 and modifications thereof | Appendix |
| 122 | hu.68 and modifications thereof | Appendix |
| 123 | AAV10 and modifications thereof | Appendix |
| 124 | AA5 and modifications thereof | Appendix |
| 125 | AAV2 and modifications thereof | Appendix |
| 126 | AAV1 and modifications thereof | Appendix |
| 127 | AAV6 and modifications thereof | Appendix |
| 128 | AAV3 and modifications thereof | Appendix |
| 129 | AAV LK03 and modifications thereof | Appendix |
| 130 | AAV7 and modifications thereof | Appendix |
| 131 | AAV8 and modifications thereof | Appendix |
| 132 | AAV hu.37 and modifications thereof | Appendix |
| 133 | AAV rh.10 and modifications thereof | Appendix |
| 134 | AA9 and modifications thereof | Appendix |
| 135 | hu.68 and modifications thereof | Appendix |
| 136 | AAV10 and modifications thereof | Appendix |
| 137 | AA5 and modifications thereof | Appendix |
| 138 | Anc80 scaffold | Appendix |
| 139 | Anc81 scaffold | Appendix |
| 140 | Anc82 scaffold | Appendix |
| 141 | Anc83 scaffold | Appendix |
| 142 | Anc84 scaffold | Appendix |
| 143 | Anc94 scaffold | Appendix |
| 144 | Anc113 scaffold | Appendix |
| 145 | Anc126 scaffold | Appendix |
| 146 | Anc127 scaffold | Appendix |
| 147 | Anc80 scaffold nucleic acid | Appendix |
| 148 | Anc81 scaffold nucleic acid | Appendix |
| 149 | Anc110 scaffold nucleic acid | Appendix |
| 150 | Anc126 scaffold nucleic acid | Appendix |
| 151 | Anc127 scaffold nucleic acid | Appendix |
| 152 | AAV2 nucleic acid | Appendix |
| 153 | AAV8-E | FIG. 8 |

### DETAILED DESCRIPTION

The current disclosure indicates that, through various points of interaction between AAV and the AAVR, the bio-distribution of the AAV (e.g., the amount of vector uptake and transduction) to and into cells in the liver and expression of an expressible nucleic acid such as a transgene in target cells can be modulated (e.g., decreased or increased), while maintaining, or even increasing, transduction of cells in other organs and peripheral tissues, such as muscle. This disclosure provides guidance on how to alter AAV capsid protein sequences to modulate the AAVR-AAV interaction in such a way that preserves the ability of AAV to engage with and bind to the AAV receptor for it to functionally enter and transduce a cell, yet changes its affinity, avidity, binding, and dissociation constants, and/or the receptor-ligand kinetics, in a manner sufficient to modify transduction, thus altering the bio-distribution of AAV to cells in the liver and in other organs and tissues, e.g., muscle tissue, within the body following in vivo administration. Based on this disclosure, the sequence of an AAV can be altered to modulate the bio-distribution of the AAV, e.g., by modulating transduction by an AAV of liver cells, without inhibiting binding of the AAV to the liver and other cells, within a patient or subject.

### Adeno Associated Virus (AAV)

Gene transfer, either for experimental or therapeutic purposes, relies upon a vector or vector system to shuttle genetic information into target cells. The vector or vector system is considered the major determinant of efficiency, specificity, host response, pharmacology, and longevity of the gene transfer reaction. Currently, the most efficient and effective way to accomplish gene transfer is through the use of vectors or vector systems based on viruses that have been made replication-defective. Some of the vectors that have shown success as gene therapy vehicles are based on adeno-associated viruses (AAVs).

Viral polypeptides can be assembled into a virus particle using, for example, a packaging host cell. The components of a virus particle (e.g., rep sequences, cap sequences, inverted terminal repeat (ITR) sequences) can be introduced into a packaging host cell using one or more viral vectors as described herein. Once assembled, virus particles can be screened for their ability to target the liver. Methods of determining the ability of an AAV to target the liver are described herein (see, for instance, Example 3).

In addition, virus particles as described herein can be screened for any number of other features or phenotypes (e.g., the ability to replicate; gene transfer properties; receptor binding ability; and/or seroprevalence in a population). In addition, methods of determining whether a virus particle binds to its receptor is known in the art, and such methods can be performed in vitro or in vivo.

Virus particles can be purified, if desired, using routine methods. As used herein, “purified” virus particles refer to virus particles that are removed from components in the mixture in which they were made such as, but not limited to, viral components (e.g., rep sequences, cap sequences), packaging host cells, and partially- or incompletely-assembled virus particles.

### AAV Liver Toggle

Previously, a particular position within the AAV VP1 capsid protein was identified that, when toggled between two different amino acids (glycine and alanine) at that position, quantitatively altered liver uptake and expression following intravenous injection of AAV in mice, non-human primates, and mice with humanized livers, as well as co-cultures of human hepatocyte (see, for example, WO 2019/217911, incorporated herein by reference in its entirety). This observation was made initially using an Anc80 AAV variant library; Anc80 is a predicted ancestral AAV scaffold sequence (see, for example, WO 2015/054653, incorporated herein by reference in its entirety). This observation is significant, because this particular residue change (G to A, or A to G) is one of the most conservative amino acid substitutions possible, yet such a conservative change still imparts very distinct liver-on/liver-off toggling.

Further work extended the relevance of this conserved toggle to other AAV viruses including natural AAV variants, AAV3B and AAV9, in which the natural variant was in “liver off” or “liver on” position, respectively. Based on this work, each liver state could be converted into the opposite state by making the indicated amino-acid substitution (see, for example, WO 2019/217911). In addition, data on the liver toggle “off” variants of Anc80 and AAV9 demonstrated that, while liver targeting is substantially reduced, uptake in non-liver tissues such as muscle is preserved quantitatively or, in certain cases, increased.

It would be appreciated by a skilled artisan that identifying and changing one or more of the “liver toggle” sequences described herein (e.g., to change an AAV from a liver-on to a liver-off, or vice versa) requires that the context of a sequence, sometimes due to the context of a resulting structural feature, be preserved. For at least that reason, the numbering of the positions referred to herein is relative to the sequence of the AAV2 VP1 protein, which is shown in SEQ ID NO:1. It would be understood, however, that any AAV, whether naturally occurring such as AAV1, AAV2, AAV3, AAV5, AAV6, AAV7, AAV8, AAV9, rh.10, hu.37, LK-03, AAV5, AAV10, and Hu68, or modified or variant such as AAV ShH10, and AAV-DJ, as well as variants such as the Anc80 AAV variant library (see, for example, WO 2015/054653) Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; Anc80DI, could be used as an unmodified sequence, i.e., reference sequence, although the numerical position may change from those referred to herein if a different reference sequence is used.

The context of a sequence, or the position of one or more amino acids in one sequence relative to another, typically is determined using a sequence alignment algorithm (e.g., Altschul et al., 1997, Nucleic Acids Res., 25:3389 3402 as incorporated into BLAST (basic local alignment search tool) programs, available at ncbi.nlm.nih.gov on the World Wide Web). BLAST or similar algorithms can be used to align two sequences (e.g., to identify the residue at a “corresponding” position, even if the two sequences differ, for example, in length), to identify motifs or consensus sequences, and/or to determine percent sequence identity between two or more sequences (nucleic acid or amino acid).

As used herein, “default parameters” used when comparing two sequences are the default parameters using the BLAST algorithm (Version BLAST+2.10.1) as implemented at blast.ncbi.nlm.nih.gov on the World Wide Web on Sep. 9, 2020. For aligning protein sequences, the default parameters are BLASTP: parameters automatically adjusted for short input sequences; expect threshold: 10; word size: 3; max matches in a query range: 0; matrix: BLOSUM62; gap costs: existence 11, extension 1; compositional adjustments: conditional compositional score matrix adjustment; and no filters or masks). For aligning nucleic acid sequences, the default parameters are BLASTN: parameters automatically adjusted for short input sequences; expect threshold: 10; word size: 28; max matches in a query range: 0; match/mismatch scores: 1,-2; gap costs: linear; filter: low complexity regions; and mask: for lookup table only.

For example, the alignments shown in FIGS. 7 and 8 of VP1 capsid proteins from a number of different AAV serotypes, or a similarly produced alignment, sometimes referred to as a “pile-up,” is one method that can be used to identify a position of an amino acid relative to, for example, AAV2 or any other unmodified AAV sequence.

### The AAV-AAVR Interaction

It has previously been shown that the majority of AAV serotypes, except AAV4 and rh.32.33, require, and thus are dependent on, the AAV receptor (AAVR) for cellular transduction (Dudek et al., 2018, 1 Virol., 92(7) pii: e02213-17). The current disclosure describes the specific changes that can be made to the AAV capsid protein sequence to modulate the AAVR-AAV interaction by altering binding (e.g., affinity and/or avidity) to provide a modified “off” rate for this interaction. These specific changes to the AAV capsid protein sequence when used in vivo dramatically alter the liver uptake of “liver on” vectors versus “liver off” vectors, suggesting that reduced affinity AAVR-AAV interactions limit the binding and eventual uptake of liver toggle off vectors in the context of vectors circulating in blood and passing through the liver.

The data described herein further indicate that, in non-liver tissues, particularly those tissues compartmentalized from the vasculature (e.g., musculature), uptake and transduction of AAV is preserved, and, in certain embodiments, increases for vectors that are liver-de-targeted. While not wishing to be bound by theory, it is believed that this may be because in those non-liver tissues, the AAVR-AAV binding affinity is less impactful to transduction than in the absence of circulation, so the AAV resides in proximity to the tissue for a longer duration, reducing the influence of the binding kinetics on eventual tissue targeting. Again, while not wishing to be bound by theory, the increased level of transduction of liver de-targeted AAVs that is observed in certain non-liver tissues may additionally and/or alternatively be due to the increased bio-distribution, since less AAV would be depleted by liver uptake.

AAVR appears to be abundantly expressed in most tissues and those levels appear to be relatively similar across most tissues. Therefore, contrary to early predictions, the abundance of AAVR expression has not been directly predictive of AAV's tissue tropism. Instead, based on the data disclosed herein, AAV tissue tropism is influenced primarily by altered AAV sequence variants, including AAV sequence variants that retain dependence on AAVR. This further suggests that the structure of AAV can influence binding to AAVR and/or the engagement of cellular co-factors involved in binding to AAVR.

### Methods of Modifying the Bio-Distribution of AAV

Changes can be introduced into an AAV nucleic acid molecule, leading to changes in the amino acid sequence of the encoded polypeptide(s). For example, changes can be introduced into nucleic acid coding sequences using mutagenesis (e.g., site-directed mutagenesis, PCR-mediated mutagenesis, CRISPR/Cas9 or other site-specific endonuclease-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

As described herein, positions Q263, S264, G265, A266, S267, N268, H271, N382, G383, S384, Q385, S446, R471, W502, T503, D528, D529, Q589, K706, or V708 in a capsid protein (numbered relative to the AAV2 VP1 capsid sequence (SEQ ID NO:1)) are involved in the binding to AAVR, and any one or more of those positions can be changed from their original amino acid, e.g., wild type or naturally occurring amino acid at that position, or the amino acid that is present at that position within a variant AAV, to modify (but not inhibit) binding between the modified AAV capsid protein and an AAV receptor (AAVR) on a liver cell in a manner that alters the AAV's ability to transduce a liver cell, thus altering the tropism or bio-distribution to the liver cells when the AAV is administered to a subject or patient and enters the circulation. Thus, changes are made in one or more of the residues at the indicated positions in a manner that results in an altered tropism of the modified (e.g., non-naturally occurring) AAV, but does not inhibit binding of the AAV to the AAVR of a given cell, which permits the liver de-targeted AAVs to bind to and transduce other types of cells in the body.

Specifically, at least positions 446, 471, and/or 708 within the VP1 capsid protein of AAV (numbered relative to the AAV2 VP1 capsid sequence (SEQ ID NO:1)) can be changed from their original, or wild type, sequence. As demonstrated in the Examples below, bio-distribution to the liver cells can be increased when the AAV capsid contains an R at position 446, an A at position 471, and/or a T at position 708, whereas bio-distribution to the liver cells can be decreased when the AAV capsid contains an S at position 446, an S at position 471, and/or an A at position 708 (all numbered relative to AAV2 (SEQ ID NO:1)). In some embodiments, a VP1 protein contains one or more of the following changes: S446N, S446R, R471A, R471S, V708T, or V708A (numbered relative to the AAV2 capsid sequence (SEQ ID NO:1)).

Specifically, at least positions 446, 471, and/or 708 within the VP1 capsid protein of AAV (numbered relative to the AAV2 VP1 capsid sequence (SEQ ID NO:1)) can be changed from their original, or wild type, sequence. As demonstrated in the Examples below, bio-distribution to the liver cells can be increased when the AAV capsid contains an R at position 446, an A at position 471, and/or a T at position 708, whereas bio-distribution to the liver cells can be decreased when the AAV capsid contains an S at position 446, an S at position 471, and/or an A at position 708 (all numbered relative to AAV2 (SEQ ID NO:1)). In some embodiments, a VP1 protein contains one or more of the following changes: S446N, S446R, R471A, R471S, V708T, or V708A (numbered relative to the AAV2 capsid sequence (SEQ ID NO:1)).

Specifically, at least positions 266, 271, and/or 446 within the VP1 capsid protein of AAV (numbered relative to the AAV2 VP1 capsid sequence (SEQ ID NO:1)) can be changed from their original, or wild type, sequence. Bio-distribution to the liver cells can be altered when the AAV capsid contains, for example, an A or a G at position 266, an H or T at position 271, and/or a S, N or R at position 446 (all numbered relative to AAV2 (SEQ ID NO:1)). In some embodiments, a VP1 protein contains one or more of the following changes: A266G, H271T, R446A, or R446S (numbered relative to the AAV2 capsid sequence (SEQ ID NO:1)).

Specifically, at least positions 471, 589, and/or 708 within the VP1 capsid protein of AAV (numbered relative to the AAV2 VP1 capsid sequence (SEQ ID NO:1)) can be changed from their original, or wild type, sequence. Bio-distribution to the liver cells can be altered when the AAV capsid contains, for example, an R, A or S at position 471, a Q or an A at position 589, and/or a V, T or A at position 708 (all numbered relative to AAV2 (SEQ ID NO:1)). In some embodiments, a VP1 protein contains one or more of the following changes: R471A, R471S, Q589A, V708T, or V708A (numbered relative to the AAV2 capsid sequence (SEQ ID NO:1)).

Specifically, at least positions 266, 446, and/or 589 within the VP1 capsid protein of AAV (numbered relative to the AAV2 VP1 capsid sequence (SEQ ID NO:1)) can be changed from their original, or wild type, sequence. As demonstrated in the Examples below, bio-distribution to the liver cells can be altered when the AAV capsid contains an A or a G at position 266, a S, N or R at position 446, and/or a Q or A at position 589 (all numbered relative to AAV2 (SEQ ID NO:1)). In some embodiments, a VP1 protein contains one or more of the following changes: A266G, S446N, S446R, or Q589R (numbered relative to the AAV2 capsid sequence (SEQ ID NO:1)).

Specifically, at least positions 271, 446, and/or 471 within the VP1 capsid protein of AAV (numbered relative to the AAV2 VP1 capsid sequence (SEQ ID NO:1)) can be changed from their original, or wild type, sequence. Bio-distribution to the liver cells can be altered when the AAV capsid contains an H or a T at position 271, an S, N or R at position 446, and/or an R, A or S at position 471 (all numbered relative to AAV2 (SEQ ID NO:1)). In some embodiments, a VP1 protein contains one or more of the following changes: H271T, S446N, S446R, R471A or R471S (numbered relative to the AAV2 capsid sequence (SEQ ID NO:1)).

Nucleic acids can be obtained or produced using any number of methods including, without limitation, chemical synthesis, recombinant nucleic acid technology, and/or the polymerase chain reaction (PCR). General PCR techniques are described, for example in PCR Primer: A Laboratory Manual (Dieffenbach & Dveksler, Eds., Cold Spring Harbor Laboratory Press, 1995), and recombinant nucleic acid techniques include, for example, restriction enzyme digestion and ligation. See, for example, Sambrook et al. (1989, Molecular Cloning; a laboratory manual, Cold Spring Harbor Laboratory Press, Cold Spring Harbor, NY).

Vectors containing nucleic acid molecules that encode polypeptides also are provided. Vectors, including expression vectors, are commercially available or can be produced by recombinant technology. A vector containing a nucleic acid molecule can have one or more elements for expression operably linked to such a nucleic acid molecule, and further can include sequences such as those encoding a selectable marker (e.g., an antibiotic resistance gene), and/or those that can be used in purification of a polypeptide (e.g., 6×His tag). Elements for expression include nucleic acid sequences that direct and regulate expression of nucleic acid coding sequences. One example of an expression element is a promoter sequence (e.g., CMV or other suitable viral promoters such as, without limitation, p5, p19, and p40). Expression elements also can include one or more of introns, enhancer sequences, response elements, or inducible elements that modulate expression of a nucleic acid molecule. Expression elements can be of bacterial, yeast, insect, mammalian, or viral origin and vectors can contain a combination of expression elements from different origins. As used herein, operably linked means that elements for expression are positioned in a vector relative to a coding sequence in such a way as to direct or regulate expression of the coding sequence.

A nucleic acid molecule, e.g., a nucleic acid molecule in a vector (e.g., an expression vector or a viral vector) can be introduced into a host cell. The term “host cell” refers not only to the particular cell(s) into which the nucleic acid molecule has been introduced, but also to the progeny or potential progeny of such a cell. Many suitable host cells are known to those skilled in the art; host cells can be prokaryotic cells (e.g., E. coli) or eukaryotic cells (e.g., yeast cells, insect cells, plant cells, mammalian cells). Representative host cells can include, without limitation, A549, WEHI, 3T3, 10T1/2, BHK, MDCK, COS 1, COS 7, BSC 1, BSC 40, BMT 10, VERO, WI38, HeLa, 293 cells, Saos, C2C12, L cells, HT1080, HepG2 and primary fibroblast, hepatocyte, and myoblast cells derived from mammals including human, monkey, mouse, rat, rabbit, and hamster. Methods for introducing nucleic acid molecules into host cells are well known in the art and include, without limitation, calcium phosphate precipitation, electroporation, heat shock, lipofection, microinjection, and viral-mediated nucleic acid transfer (e.g., transduction).

### Toggle Sites

The present disclosure provides specific residues of AAV capsid proteins that can be modified to change targeting in vivo when rAAV is administered to a mammalian subject. Modification of the specific residues can alter transduction of target cells and/or transgene expression in target cells. The specific residues are referred to as toggle sites.

The toggle sites comprise amino acid residues involved in interactions between AAV capsid protein and AAVR, specifically at Q263, S264, G265, A266, S267, N268, H271, N382, G383, S384, Q385, S446, R471, W502, T503, D528, D529, Q589, K706, and V708, wherein the amino acid position is numbered relative to SEQ ID NO: 1 (AAV2 VP1). FIG. 7, FIG. 8 and FIG. 9 show the respective toggle sites highlighted on the amino acid sequence of various unmodified AAV VP1 capsid proteins.

Target-specific tropism of AAVs can be altered by changing one or more amino acids at the toggle sites. A modified capsid protein (e.g., modified VP1 capsid) provided herein includes one or more amino acid difference in the toggle sites—Q263, S264, G265, A266, S267, N268, H271, N382, G383, S384, Q385, S446, R471, W502, T503, D528, D529, Q589, K706, and V708—compared to an unmodified capsid protein.

In some embodiments, the modification is introduced at selected toggle sites consisting of S446, R471 and V708. In some embodiments, the modification is not at A266.

Modification of the toggle sites can alter biodistribution of the modified rAAV. In some embodiments, the modification increases transduction of target cells compared to an unmodified rAAV. In some embodiments, the modification increases expression of an expressible polynucleotide delivered by the modified rAAV compared to an unmodified rAAV in target cells. In some embodiments, the modification decreases transduction of target cells compared to an unmodified rAAV. In some embodiments, the modification decreases expression of an expressible polynucleotide delivered by the modified rAAV compared to an unmodified rAAV in target cells. In some embodiments, the target is liver and the target cells are hepatocytes. In some embodiments, the target is not liver.

### Modified AAV Capsid Protein

Modified capsid proteins of the present disclosure comprise means for altering rAAV biodistribution following administration to a mammalian subject as compared to biodistribution of an unmodified rAAV comprising VP1, VP2, and VP3 capsid proteins having amino acid sequences identical to those of the modified rAAV except for said means. rAAVs comprising the modified capsid proteins are referred to as modified rAAVs.

The means can alter the biodistribution when administered locally or systemically. In some embodiments, the means alter biodistribution when intravenously infused.

In some embodiments, the means for altering rAAV biodistribution change interaction of the modified rAAV with AAVR expressed on the mammalian subject's cells. For example, the means for altering rAAV biodistribution reduce or increase interaction of the modified rAAV with AAVR. In some embodiments, the means change binding affinity or binding stability between the modified AAV capsid protein and AAVR compared to an unmodified AAV capsid protein and AAVR. In some embodiments, the means comprise changes in the interaction interface between AAV capsid protein (VP1, VP2, or VP3 capsid protein) and AAVR.

In some embodiments, the means for altering rAAV biodistribution comprise a presence or absence of certain amino acid residues at one or more positions selected from the group consisting of Q263, S264, G265, A266, S267, N268, H271, N382, G383, S384, Q385, S446, R471, W502, T503, D528, D529, Q589, K706, and V708, wherein the amino acid position is numbered relative to SEQ ID NO: 1 (AAV2 VP1). In some embodiments, more than one amino acids can be substituted, inserted, and/or deleted at one or more positions selected from the group consisting of Q263, S264, G265, A266, S267, N268, H271, N382, G383, S384, Q385, S446, R471, W502, T503, D528, D529, Q589, K706, and V708 to introduce the means for altering rAAV biodistribution.

In some embodiments, the means for altering rAAV biodistribution comprises one or more amino acid substitution, insertion and/or deletion at one or more of the toggle sites—Q263, S264, G265, A266, S267, N268, H271, N382, G383, S384, Q385, S446, R471, W502, T503, D528, D529, Q589, K706, and V708. The amino acid substitution, insertion and/or deletion can change one or more original amino acid residues at a toggle site to one or more different amino acid residues that occur at the same toggle site of a different AAV capsid as provided in Tables 1A, 1B, 1C and 2. For example, the Q263 toggle site (relative to AAV2) can be changed from Q to any of A, E, T or G. The Q264 toggle site of AAV1 can be changed from S to any of G, T, A or V. Toggle sites where one or more amino acid substitution, insertion and/or deletion can be introduced are highlighted with boxes in FIG. 7 and FIG. 8.

In some embodiments, a modified VP1 capsid protein has a sequence shown in SEQ ID NO: 112-137. In some embodiments, a modified VP1 capsid protein has a sequence having at least 95%, 96%, 97%, 98%, 99%, or 95.5% identity to one of the sequences shown in SEQ ID NO: 112-137.

In some embodiments, a modified VP1 capsid protein has one of the sequences shown in SEQ ID NO: 2-100, 138-146 or 153 with one or more amino acid substitutions, insertions, and/or deletions at one of the toggle sites—Q263, S264, G265, A266, S267, N268, H271, N382, G383, S384, Q385, S446, R471, W502, T503, D528, D529, Q589, K706, and V708, wherein the amino acid position is numbered relative to SEQ ID NO: 1 (AAV2 VP1). In some embodiments, a modified VP1 capsid protein has one of the sequences shown in SEQ ID NO: 2-100, 138-146 or 153 with one or more amino acid substitutions, insertions, and/or deletions at one of the selected toggle sites—S446, R471 and V708, wherein the amino acid position is numbered relative to SEQ ID NO: 1 (AAV2 VP1).

TABLE 1A

| Position | AAV2 | AAV1 | AAV6 | AAV3 | AAVLK03 | AAV7 | AAV8 | AAVhu.37 | AAVrh.10 | AAV9 | AAVhu.68 | AAV10 | AAV5 |
|---|---|---|---|---|---|---|---|---|---|---|---|---|---|
| 263 | Q | A | A | Q | Q | E | T | T | T | T | T | T | G |
| 264 | S | S | S | S | S | T | S | S | S | S | S | S | S |
| 265 | G | T | T | G | G | A | G | G | G | G | G | G | V |
| 266 | A | G | G | A | A | G | G | G | G | G | G | G | D |
| 267 | S | S | S | S | S | T | T | T | T | S | S | T | S |
| 268 | N | N | N | N | N | N | N | N | N | N | N | N | N |
| 271 | H | H | H | H | H | T | T | T | T | A | A | T | A |
| 382 | N | N | N | N | N | N | N | N | N | D | D | N | N |
| 383 | G | G | G | G | G | G | G | G | G | G | G | G | T |
| 384 | S | S | S | S | S | S | S | S | S | S | S | S | E |
| 385 | Q | Q | Q | Q | Q | Q | Q | Q | Q | Q | Q | Q | N |
| 446 | S | N | N | N | N | A | S | S | S | S | S | S | V |
| 471 | R | S | S | S | S | A | A | S | S | A | A | S | A |
| 502 | W | W | W | W | W | W | W | W | W | W | W | W | F |
| 503 | T | T | T | T | T | T | T | T | T | P | P | T | A |
| 528 | D | D | D | D | D | D | D | D | D | E | E | D | L |
| 529 | D | D | D | D | D | D | D | D | D | G | G | D | Q |
| 589 | Q | D | D | A | A | A | A | G | A | Q | Q | G | A |
| 706 | K | K | K | K | K | K | K | K | K | K | K | K | D |
| 708 | V | A | A | V | V | T | T | T | T | N | N | T | Q |

TABLE 1B

| Position | AAV2 | Anc80 | Anc81 | Anc82 | Anc83 | Anc84 | Anc94 | Anc110 | Anc113 | Anc126 | Anc127 |
|---|---|---|---|---|---|---|---|---|---|---|---|
| 263 | Q | Q | Q | T | T | T | T | T | Q | Q | Q |
| 264 | S | S | S | S | S | S | S | S | S | S | S |
| 265 | G | G | G | G | G | G | G | G | A | G | G |
| 266 | A | A | G | G | G | G | G | G | G | A | A |
| 267 | S | S | T | T | T | T | T | T | T | S | S |
| 268 | N | N | N | N | N | N | N | N | N | N | N |
| 271 | H | T | T | T | T | T | T | T | T | H | H |
| 382 | N | N | N | N | N | N | N | N | N | N | N |
| 383 | G | G | G | G | G | G | G | G | G | G | G |
| 384 | S | S | S | S | S | S | S | S | S | S | S |
| 385 | Q | Q | Q | Q | Q | Q | Q | Q | Q | Q | Q |
| 446 | S | S | S | S | S | S | S | S | A | S | S |
| 471 | R | A | A | A | A | S | S | A | A | A | A |
| 502 | W | W | W | W | W | W | W | W | W | W | W |
| 503 | T | T | T | T | T | T | T | T | T | T | T |
| 528 | D | D | D | D | D | D | D | D | D | D | D |
| 529 | D | D | D | D | D | D | D | D | D | D | D |
| 589 | Q | A | A | A | A | A | A | Q | A | A | A |
| 706 | K | K | K | K | K | K | K | K | K | K | K |
| 708 | V | T | T | T | T | T | T | T | T | A | V |

TABLE 1C

| Position | AAV2 | Anc80L27 | Anc80L59 | Anc80L60 | Anc80L62 | Anc80L65 | Anc80L33 | Anc80L36 | Anc80L44 | Anc80L1 |
|---|---|---|---|---|---|---|---|---|---|---|
| 263 | Q | Q | Q | Q | Q | Q | Q | Q | Q | Q |
| 264 | S | S | S | S | S | S | S | S | S | S |
| 265 | G | G | G | G | G | G | G | G | G | G |
| 266 | A | G | A | G | G | G | G | G | G | A |
| 267 | S | T | T | T | T | T | T | T | T | T |
| 268 | N | N | N | N | N | N | N | N | N | N |
| 271 | H | T | T | T | T | T | T | T | T | T |
| 382 | N | N | N | N | N | N | N | N | N | N |
| 383 | G | G | G | G | G | G | G | G | G | G |
| 384 | S | S | S | S | S | S | S | S | S | S |
| 385 | Q | Q | Q | Q | Q | Q | Q | Q | Q | Q |
| 446 | S | S | S | S | S | S | S | S | S | S |
| 471 | R | A | A | A | A | A | A | A | A | A |
| 502 | W | W | W | W | W | W | W | W | W | W |
| 503 | T | T | T | T | T | T | T | T | T | T |
| 528 | D | D | D | D | D | D | D | D | D | D |
| 529 | D | D | D | D | D | D | D | D | D | D |
| 589 | Q | A | A | A | A | A | A | A | A | A |
| 706 | K | K | K | K | K | K | K | K | K | K |
| 708 | V | T | T | T | T | T | T | T | T | T |

In some embodiments the means for altering rAAV biodistribution comprises one or more amino acid substitutions, insertions, and/or deletions at more than one of the toggle sites. In some embodiments the means for altering rAAV biodistribution comprises one or more amino acid substitution, insertion, and/or deletion at two of the toggle sites. In some embodiments the means for altering rAAV biodistribution comprises one or more amino acid substitution, insertion, and/or deletion at three of the toggle sites. In some embodiments the means for altering rAAV biodistribution comprises one or more amino acid substitution, insertion, and/or deletion at four, five, six, seven, eight, nine, ten, eleven, twelve, thirteen, fourteen, fifteen, sixteen, seventeen, eighteen, nineteen or twenty of the toggle sites. In some embodiments, the means for altering rAAV biodistribution comprises one or more amino acid substitution, insertion, and/or deletion at A2666 and one or more additional amino acid substitutions, insertion and/or deletion at other toggle sites—Q263, S264, G265, S267, N268, H271, N382, G383, S384, Q385, S446, R471, W502, T503, D528, D529, Q589, K706, and V708. In some embodiments, the means for altering rAAV biodistribution does not comprise an amino acid substitution at A266.

In some embodiments, the means for altering rAAV biodistribution comprises one or more amino acid substitutions at the selected toggle sites—S446, R471 and V708. In some embodiments, the means for altering rAAV biodistribution comprises two amino acid substitutions or three amino acid substitutions at the selected toggle sites. In some embodiments, the modified VP1 capsid protein comprises one, two or three amino acid residues selected from the group consisting of 446R, 471A and 708T. In some embodiments, the modified VP1 capsid protein comprises one, two or three amino acid residues selected from the group consisting of 446S, 471S and 708A.

In some embodiments, a modified AAV capsid protein differs from the unmodified AAV capsid protein having greatest sequence identity to the modified AAV capsid protein VP1 protein, as aligned using default parameters. In some embodiments, a modified AAV capsid protein differs from the unmodified AAV capsid protein with greatest sequence identity only at the toggle sites, Q263, S264, G265, A266, S267, N268, H271, N382, G383, S384, Q385, S446, R471, W502, T503, D528, D529, Q589, K706, and V708, wherein the amino acid position is numbered relative to SEQ ID NO: 1 (AAV2 VP1). In some embodiments, a modified AAV capsid protein differs from the unmodified AAV capsid protein with greatest sequence identity only at the selected toggle sites, S446, R471 and V708, wherein the amino acid position is numbered relative to SEQ ID NO: 1 (AAV2 VP1).

In some embodiments, a modified AAV capsid protein differs from the unmodified rAAV capsid protein with greatest sequence identity at the toggle sites as well as outside of the toggle sites. In some embodiments, a modified AAV capsid protein has 95%, 96%, 97%, 98%, 99% or 99.5% sequence identity to the unmodified AAV capsid protein with greatest sequence identity.

In one aspect, the present disclosure provides a modified polynucleotide encoding a modified AAV capsid protein. The modified polynucleotide encoding a modified AAV capsid protein can have one or more nucleotide substitution, insertion or deletion at one or more positions selected from the group consisting of (787-789), (790-792), (793-795), (796-798), (799-801), (802-804), (811-813), (1144-1146), (1150-1152), (1153-1155), (1336-1338), (1411-1413), (1504-1506), (1507-1509), (1582-1584), (1585-1587), (1765-1767), (2116-2118), or (2122-2124) compared to an unmodified polynucleotide encoding an unmodified AAV capsid, wherein the nucleotide position is numbered relative to SEQ ID NO: 152 (AAV2 VP1). The nucleotide substitution, insertion or deletion can introduce one or more amino acid substitution, insertion and/or deletion at one or more of the toggle sites—Q263, S264, G265, A266, S267, N268, H271, N382, G383, S384, Q385, S446, R471, W502, T503, D528, D529, Q589, K706, and V708 to the capsid protein encoded by the polynucleotide.

In some embodiments, the modified polynucleotide comprises one of the sequences shown in SEQ ID NO: 147-151 with one or more nucleotide substitution, insertion or deletion at one or more positions selected from the group consisting of (787-789), (790-792), (793-795), (796-798), (799-801), (802-804), (811-813), (1144-1146), (1150-1152), (1153-1155), (1336-1338), (1411-1413), (1504-1506), (1507-1509), (1582-1584), (1585-1587), (1765-1767), (2116-2118), or (2122-2124), wherein the nucleotide position is numbered relative to SEQ ID NO: 152 (AAV2 VP1).

In one aspect, the present disclosure provides a modified polynucleotide encoding a modified AAV capsid protein. The modified polynucleotide encoding a modified AAV capsid protein can have one or more nucleotide substitution, insertion or deletion at one or more positions selected from the group consisting of (1336-1338), (1411-1413) or (2122-2124) compared to an unmodified polynucleotide encoding an unmodified AAV capsid, wherein the nucleotide position is numbered relative to SEQ ID NO: 152 (AAV2 VP1). The nucleotide substitution, insertion or deletion can introduce one or more amino acid substitution, insertion and/or deletion at one or more of the selected toggle sites—S446, R471 and V708 to the capsid protein encoded by the polynucleotide. The nucleotide substitution, insertion or deletion can introduce one or more amino acid substitution, insertion and/or deletion at one or more of the selected toggle sites—A266, S446, R471 and V708.

In some embodiments, the modified polynucleotide comprises one of the sequences shown in SEQ ID NO: 147-151 with one or more nucleotide substitution, insertion or deletion at one or more positions selected from the group consisting of (1336-1338), (1411-1413) or (2122-2124), wherein the nucleotide position is numbered relative to SEQ ID NO: 152 (AAV2 VP1).

In another aspect, the present disclosure provides a vector comprising the modified polynucleotide encoding a modified AAV capsid protein described herein. In some embodiments, the vector is a plasmid.

Modified Recombinant AAV (Modified rAAV)

The present disclosure further provides modified rAAVs comprising a modified AAV capsid protein (VP1, VP2 or VP3 capsid protein) disclosed herein and a recombinant nucleic acid vector.

In some embodiments, the modified rAAV achieves higher transduction of liver following administration to a mammalian subject as compared to an unmodified rAAV comprising an unmodified VP1 capsid protein having greatest sequence identity to the modified VP1 administered by the same route of administration. In some embodiments, the modified rAAV achieves higher expression in liver of an expressible polynucleotide within the recombinant nucleic acid genome following administration to a mammalian subject as compared to expression of the expressible polynucleotide administered in an unmodified rAAV comprising a VP1 capsid protein having greatest sequence identity to the modified VP1, administered by the same route of administration.

In some embodiments, the modified rAAV achieves lower transduction of liver following administration to a mammalian subject as compared to an unmodified rAAV comprising a VP1 capsid protein having greatest sequence identity to the modified VP1, administered by the same route of administration. In some embodiments, the modified rAAV achieves lower expression in liver of an expressible polynucleotide within the recombinant nucleic acid genome following administration to a mammalian subject as compared to the expressible polynucleotide administered in an unmodified rAAV comprising a VP1 capsid protein having greatest sequence identity to the modified VP1 administered by the same route of administration.

In some embodiments, the modified rAAV achieves higher transduction of an organ outside of liver following administration to a mammalian subject as compared to an unmodified rAAV comprising an unmodified VP1 capsid protein having greatest sequence identity to the modified VP1, administered by the same route of administration. In some embodiments, the modified rAAV achieves higher expression in an organ outside of liver of an expressible polynucleotide within the recombinant nucleic acid genome as compared to the expressible polynucleotide delivered in an unmodified rAAV comprising an unmodified VP1 capsid protein having greatest sequence identity to the modified VP1 administered by the same route of administration.

In some embodiments, the modified rAAV achieves lower transduction of an organ outside of liver of an expressible polynucleotide within the recombinant nucleic acid genome as compared to the expressible polynucleotide delivered in an unmodified rAAV comprising an unmodified VP1 capsid protein having greatest sequence identity to the modified VP1 administered by the same route of administration. In some embodiments, the modified rAAV achieves lower expression in an organ outside of liver of an expressible polynucleotide within the recombinant nucleic acid genome following administration to a mammalian subject as compared to the expressible polynucleotide administered in an unmodified rAAV comprising a VP1 capsid protein having greatest sequence identity to the modified VP1 administered by the same route of administration.

In some embodiments, the modified rAAV has reduced interaction with AAVR expressed on the mammalian subject's cells as compared to an unmodified rAAV comprising an unmodified VP1 capsid protein having greatest sequence identity to the modified VP1 administered by the same route of administration. In some embodiments, the modified rAAV has greater interaction with AAVR expressed on the mammalian subject's cells as compared to an unmodified rAAV comprising an unmodified VP1 capsid protein having greatest sequence identity to the modified VP1 delivered by the same route of administration.

In some embodiments, the modified rAAV has less liver toxicity than an unmodified rAAV comprising an unmodified VP1 capsid protein having greatest sequence identity to the modified VP1 administered by the same route of administration and in the same dose.

Pharmaceutical Composition Comprising Modified rAAV

In one aspect, the present disclosure provides a pharmaceutical composition comprising a modified rAAV of the present disclosure and a pharmaceutically acceptable carrier. The modified rAAV comprises a modified AAV capsid protein as described herein and a recombinant nucleic acid vector containing an expressible polynucleotide.

The pharmaceutical composition can be used to deliver the recombinant nucleic acid vector to a target within a mammalian subject. When the pharmaceutical composition is administered, the modified rAAV can achieve a higher transduction of target cells following administration to a mammalian subject as compared to an unmodified rAAV comprising an unmodified VP1 capsid protein having greatest sequence identity to the modified VP1 administered by the same route of administration and in the same dose. In some embodiments, the modified rAAV achieves higher expression in target cells of an expressible polynucleotide within the recombinant nucleic acid genome following administration to a mammalian subject as compared to the expressible polynucleotide administered in an unmodified rAAV comprising a VP1 capsid protein having greatest sequence identity to the modified VP1 administered by the same route of administration and in the same dose.

Targeting of modified rAAVs can be tested in an experimental animal by measuring rAAV transduction or expression of an expressible polynucleotide. In some embodiments, targeting is measured in a non-human primate (NHP), mice, rats, birds, rabbits, guinea pigs, hamsters, farm animals (including pigs and sheep), dogs, or cats.

Targeting of modified rAAVs can be measured after systemic or local administration of rAAVs. In some embodiments, targeting of modified rAAVs is measured after intravenous infusion of rAAVs.

In some embodiments, the modified rAAV achieves 2-fold, 3-fold, 4-fold, 5-fold, 10-fold, 20-fold, 30-fold, 40-fold, 50-fold, 100-fold, 200-fold, 500-fold, 750-fold, 1000-fold, or 2500-fold reduction in gene transfer to the liver or in viral infection or transgene expression in liver cells after a first administration into a mammalian subject compared to an unmodified rAAV. In some embodiments, the modified rAAV achieves 2-fold, 3-fold, 4-fold, 5-fold, 10-fold, 20-fold, 30-fold, 40-fold, 50-fold, 100-fold, 200-fold, 500-fold, 750-fold, 1000-fold, or 2500-fold reduction in gene transfer to the liver or in viral infection or transgene expression in liver cells after a first administration into a rhesus macaque.

In some embodiments, the modified rAAV achieves at least 10-fold increase in gene transfer to the liver or in viral infection or transgene expression in liver cells after a first administration into a mammalian subject compared to an unmodified rAAV. In some embodiments, the modified rAAV achieves at least 10-fold increase in gene transfer to the liver or in viral infection or transgene expression in liver cells after a first administration into a rhesus macaque.

In some embodiments, the modified rAAV achieves at least 10-fold reduction in gene transfer to the liver or in viral infection or transgene expression in liver cells after a first administration into a mammalian subject compared to an unmodified rAAV. In some embodiments, the modified rAAV achieves at least 10-fold reduction in gene transfer to the liver or in viral infection or transgene expression in liver cells after a first administration into a rhesus macaque.

In some embodiments, the modified rAAV achieves at least 10-fold increase in gene transfer to the liver or in viral infection or transgene expression in liver cells after a first administration into a mammalian subject compared to an unmodified rAAV. In some embodiments, the modified rAAV achieves at least 10-fold increase in gene transfer to the liver or in viral infection or transgene expression in liver cells after a first administration into a rhesus macaque.

In some embodiments, the modified rAAV achieves at least 100-fold reduction in gene transfer to the liver or in viral infection or transgene expression in liver cells after a first administration into a mammalian subject compared to an unmodified rAAV. In some embodiments, the modified rAAV achieves at least 100-fold reduction in gene transfer to the liver or in viral infection or transgene expression in liver cells after a first administration into a rhesus macaque.

In some embodiments, the modified rAAV achieves at least 100-fold increase in gene transfer to the liver or in viral infection or transgene expression in liver cells after a first administration into a mammalian subject compared to an unmodified rAAV. In some embodiments, the modified rAAV achieves at least 100-fold increase in gene transfer to the liver or in viral infection or transgene expression in liver cells after a first administration into a rhesus macaque.

In some embodiments, the modified rAAV achieves at least 1000-fold reduction in gene transfer to the liver or in viral infection or transgene expression in liver cells after a first administration into a mammalian subject compared to an unmodified rAAV. In some embodiments, the modified rAAV achieves at least 1000-fold reduction in gene transfer to the liver or in viral infection or transgene expression in liver cells after a first administration into a rhesus macaque.

In some embodiments, the modified rAAV achieves at least 1000-fold increase in gene transfer to the liver or in viral infection or transgene expression in liver cells after a first administration into a mammalian subject compared to an unmodified rAAV. In some embodiments, the modified rAAV achieves at least 1000-fold increase in gene transfer to the liver or in viral infection or transgene expression in liver cells after a first administration into a rhesus macaque.

### Methods of Using Liver-Toggled Viruses

A non-naturally occurring, modified AAV virus as described herein (e.g., in which the VP1 capsid protein sequence is changed or engineered to exhibit the desired bio-distribution (e.g., liver-on or liver off)) can be used in a number of research and/or therapeutic applications. For example, a liver-on or liver-off virus can be used in human or animal medicine for gene therapy (e.g., in a vector or vector system for gene transfer) or for vaccination (e.g., for antigen presentation). More specifically, a liver-on or liver-off virus can be used for gene addition, gene augmentation, genetic delivery of a polypeptide therapeutic, genetic vaccination, gene silencing, genome editing, gene therapy, RNAi delivery, cDNA delivery, mRNA delivery, miRNA delivery, miRNA sponging, genetic immunization, optogenetic gene therapy, transgenesis, DNA vaccination, or DNA immunization of liver cells or non-liver cells.

A non-naturally occurring, modified AAV in which the sequence of the capsid protein has been changed to alter the bio-distribution of the AAV (e.g., the liver bio-distribution) can include a transgene (in cis or trans configuration with other viral sequences). A transgene can be, for example, a reporter gene (e.g., beta-lactamase, beta-galactosidase (LacZ), alkaline phosphatase, thymidine kinase, green fluorescent polypeptide (GFP), chloramphenicol acetyltransferase (CAT), or luciferase, or fusion polypeptides that include an antigen tag domain such as hemagglutinin or Myc), or a therapeutic gene (e.g., genes encoding hormones or receptors thereof, growth factors or receptors thereof, differentiation factors or receptors thereof, immune system regulators (e.g., cytokines and interleukins) or receptors thereof, enzymes, RNAs (e.g., inhibitory RNAs or catalytic RNAs), or target antigens (e.g., oncogenic antigens, autoimmune antigens)).

The particular transgene will be selected depending, at least in part, on the particular disease or deficiency being treated. Simply by way of example, gene transfer or gene therapy can be applied to the treatment of hemophilia, retinitis pigmentosa, cystic fibrosis, leber congenital amaurosis, lysosomal storage disorders, inborn errors of metabolism (e.g., inborn errors of amino acid metabolism including phenylketonuria, inborn errors of organic acid metabolism including propionic acidemia, inborn errors of fatty acid metabolism including medium-chain acyl-CoA dehydrogenase deficiency (MCAD)), cancer, achromatopsia, cone-rod dystrophies, macular degenerations (e.g., age-related macular degeneration), lipopolypeptide lipase deficiency, familial hypercholesterolemia, spinal muscular atrophy, Duchenne's muscular dystrophy, Alzheimer's disease, Parkinson's disease, obesity, inflammatory bowel disorder, diabetes, congestive heart failure, hypercholesterolemia, hearing loss, coronary heart disease, familial renal amyloidosis, Marfan's syndrome, fatal familial insomnia, Creutzfeldt-Jakob disease, sickle-cell disease, Huntington's disease, fronto-temporal lobar degeneration, Usher syndrome, lactose intolerance, lipid storage disorders (e.g., Niemann-Pick disease, type C), Batten disease, choroideremia, glycogen storage disease type II (Pompe disease), ataxia telangiectasia (Louis-Bar syndrome), congenital hypothyroidism, severe combined immunodeficiency (SCID), and/or amyotrophic lateral sclerosis (ALS). A transgene also can be, for example, an immunogen that is useful for immunizing a subject (e.g., a human, an animal (e.g., a companion animal, a farm animal, an endangered animal). For example, immunogens can be obtained from an organism (e.g., a pathogenic organism) or an immunogenic portion or component thereof (e.g., a toxin polypeptide or a by-product thereof). By way of example, pathogenic organisms from which immunogenic polypeptides can be obtained include viruses (e.g., picornavirus, enteroviruses, orthomyxovirus, reovirus, retrovirus), prokaryotes (e.g., Pneumococci, Staphylococci, Listeria, Pseudomonas), and eukaryotes (e.g., amebiasis, malaria, leishmaniasis, nematodes). It would be understood that the methods described herein and compositions produced by such methods are not to be limited by any particular transgene.

### Administration of AAV

A liver-on or a liver-off AAV vector, usually suspended in a physiologically compatible carrier, can be administered to a subject (e.g., a human or non-human mammal). Suitable carriers include saline, which may be formulated with a variety of buffering solutions (e.g., phosphate buffered saline), lactose, sucrose, calcium phosphate, gelatin, dextran, agar, pectin, and water. A viral vector typically is administered in sufficient amounts to transduce or infect the desired cells and to provide sufficient levels of gene transfer and expression to provide a therapeutic benefit without undue adverse effects. Conventional and pharmaceutically acceptable routes of administration include, but are not limited to, direct delivery to an organ such as, for example, the liver or lung, orally, intranasally, intratracheally, intrathecally, by inhalation, intravenously, intramuscularly, intraocularly, subcutaneously, intradermally, transmucosally, or by other routes of administration. Routes of administration can be combined, if desired.

The dose of a viral vector administered to a subject will depend primarily on factors such as the condition being treated, and the age, weight, and health of the subject. For example, a therapeutically effective dosage of a viral vector to be administered to a human subject generally is in the range of from about 0.1 ml to about 10 ml of a solution containing concentrations of from about 1×10e1 to 1×10e12 genome copies (GCs) of viruses (e.g., about 1×10e3 to 1×10e9 GCs). Transduction and/or expression of a transgene can be monitored at various time points following administration by DNA, RNA, or protein assays. In some instances, the levels of expression of the transgene can be monitored to determine the frequency and/or amount of dosage. Dosage regimens similar to those described for therapeutic purposes also may be utilized for immunization.

A second “corresponding” subject (e.g., mammalian subject), as used herein, refers to a subject that is the same type (e.g., species and, where applicable, breed or strain) as the first subject, and does not substantially differ from the first subject in AAV transduction.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—the Anc AAV Capsid Libraries

Libraries of the AAV capsid were previously generated (referred to as Anc AAVs) based on ancestral sequence reconstruction. Anc AAVs approximate a putative ancestral state of an AAV phylogeny. This work was based on recreating ancestors along a putative phylogeny of the majority of known primate AAVs including AAV1, AAV2, AAV3, AAV6, AAV7, AAV8, and AAV9 (but not AAV4 or AAV5). This process inferred a probability score (posterior probability) to each of the 20 amino acids at each of the positions along the AAV capsid protein VP1. These approaches were described in Zinn et al. (2015, Cell Reports, 12(6):1056-68) and WO 2015/054653, and were used to generate two Anc libraries, referred to as Anc126 and Anc127.

These Anc126 and Anc127 variant libraries were constructed using molecular cloning and DNA synthesis in a pooled format, i.e., all variants of a particular library were parallel synthetized in the same receptacle. The design of this library plasmid was such that it encodes the viral capsid sequence within the AAV vector genome. In addition, the design of these libraries includes a short DNA identifier or barcode that allows for efficient and high throughput next generation sequencing (NGS) using short read NGS platforms such as those sold by Illumina. In the experiments outlined herein, each barcode identified a single AAV capsid variant and each capsid variant was identified by a unique barcode.

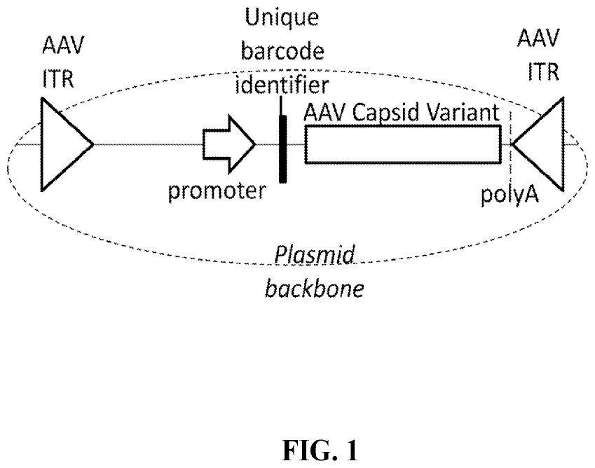

FIG. 1 illustrates a minimal design of a library construct for AAV variants within pooled barcoded library. Elements of an AAV library plasmid construct are AAV Inverted Terminal Repeat (ITR), one or more promoter(s) or poly adenylation signal(s) (polyA), which can be from AAV or heterologous to AAV, an AAV capsid variant open reading frame (ORF), and a barcode identifier for the capsid. It would be understood that variations on the construct shown in FIG. 1 are possible, such as the presence of the barcode outside of the expression cassette yet within the ITRs, one promoter driving the capsid gene and another a transcript with the barcode, and/or various elements for promoter(s) and polyA signal(s).

This pooled plasmid library was used to generate a viral vector library by transfection into HEK293 cells together with an adenoviral helper gene plasmid construct and a plasmid containing the AAV rep expression cassette. Importantly, this transfection was performed under low plasmid concentration conditions to maximize the degree of “self-packaging,” i.e., the packaging of a viral genome for a particular capsid within the capsid. The viral library was next assessed for its diversity using Illumina NGS with a focused sequencing of the barcoding region. This data provided a count for each barcode identified within the NGS sample of the viral library preparation and, thereby, gave a relative representation of the barcode (and hence capsid variant) diversity. NGS sequencing of the barcode indicated that both of the Anc126 and Anc127 libraries were sufficiently diverse and representative across the relevant positions of sequence variation.

### Example 2—Summary of Sequence Diversity Across the AAVR Footprint

FIG. 2 is an alignment of Anc126 (SEQ ID NO:99) and Anc127 (SEQ ID NO:100) capsid protein sequences relative to the AAV2 VP1 capsid protein sequence (SEQ ID NO:1) using the MUSCLE algorithm (Edgar, R. C. (2004) MUSCLE: multiple sequence alignment with high accuracy and high throughput Nucleic Acids Res. 32(5):1792-1797) with the following parameters: distance measure kmer6_6, clustering method UPGMB, tree rooting method pseudo, distance weighting CLUSTALW, anchor spacing 32, open gap penalty −1. The residues predicted to be located within the AAVR footprint are boxed. Additionally, Table 2 below provides each of the predicted AAVR footprint residues in AAV2 and the corresponding residue in Anc80, Anc126, and Anc127, with dots (“.”) representing conserved amino acids. Positions at which alternate residues were observed are shown in Table 2, indicating that these sites of variation were ambiguous in the Anc library.

TABLE 2
The residue and positions on the AAV VP1 capsid protein
predicted to be involved in AAVR-AAV binding

| Position | AAV2 | Anc80 | Anc126 | Anc127 |
|---|---|---|---|---|
| 263 | Q | . | . | . |
| 264 | S | . | . | . |
| 265 | G | . | . | . |
| 266 | A | A/G | . | . |
| 267 | S | . | . | . |
| 268 | N | . | . | . |
| 271 | H | T | . | . |
| 382 | N | . | . | . |
| 383 | G | . | . | . |
| 384 | S | . | . | . |
| 385 | Q | . | . | . |
| 446 | S | . | N/S | R/S |
| 471 | R | A | A/S | A/S |
| 502 | W | . | . | . |
| 503 | T | . | . | . |
| 528 | D | . | . | . |
| 529 | D | . | . | . |
| 589 | Q | A | A | A |
| 706 | K | . | . | . |
| 708 | V | T | T/A | . |

### Example 3—Identifying Sequences Associated with Liver Uptake

The AAV126 and AAV127 variant libraries were injected into C57Bl/6 mice via an intravenous route at a dose of 6.2e10 GCs total to interrogate the impact of the variant sites within the library on liver tropism, especially those sites identified in Table 2, which correspond to the AAVR footprint. Animals were sacrificed and tissues were harvested at 28 days following injection. Tissues were subjected to DNA isolation, after which the DNA samples were amplified using PCR with primers flanking the barcode sequence. PCR amplicons underwent a second amplification to incorporate Illumina NGS indexes. These NGS samples were subsequently sequenced using Illumina NGS instrumentation MiSeq® or NovaSeq®. Data was subsequently analyzed for each variant to represent enrichment of barcodes in liver as compared to the input injected virus using the barcode counts of the liver versus those in the input viral preparation. A quantitative readout of the liver tissue was obtained, which indicated the extent of liver distribution of each member of the respective library.

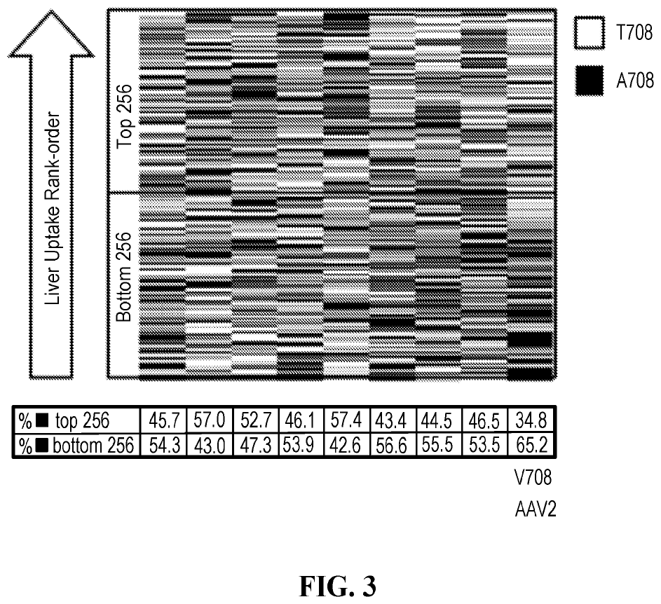

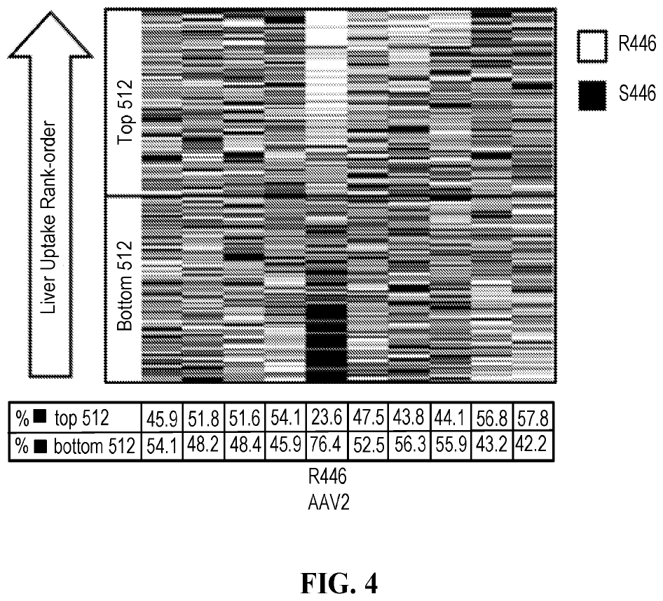

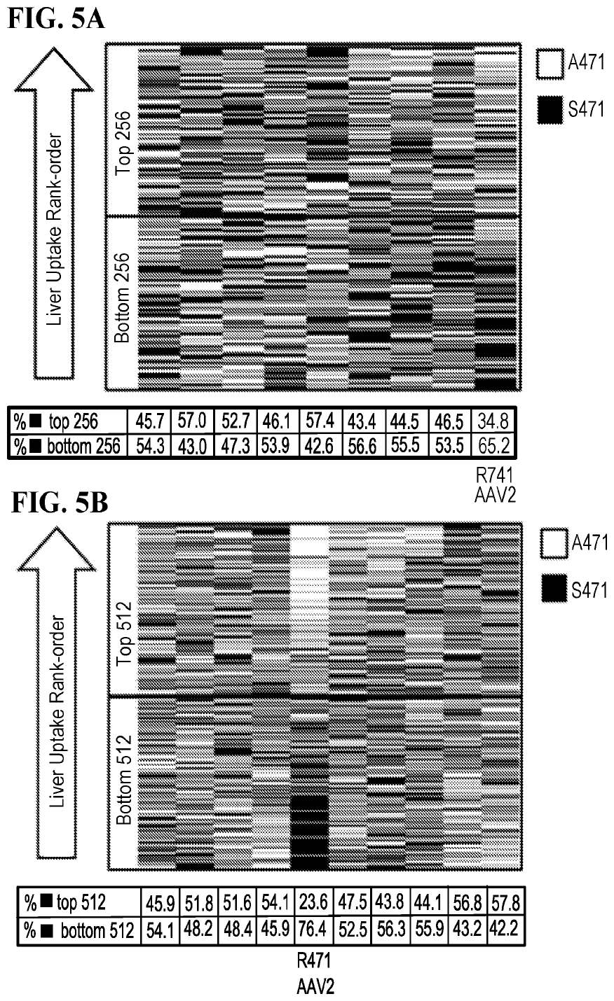

FIG. 3 and FIG. 4 represent the relative performance data in the liver for Anc126 and Anc127 members, respectively, as does FIGS. 5A and 5B. The analysis provided here, in the form of graphs referred to as a “fingerprint plots,” illustrates the performance for all members of the Anc126 or Anc127 vector libraries in rank order from top to bottom. In addition, columns represent the sites of variation within the Anc126 or Anc127 library design. Each site of variation is ambiguous, i.e., has two different residues that can be encoded in that position.

Data in FIG. 3 indicates that position P9, which corresponds to residue 708 in AAV2 (SEQ ID NO:1), is the only position in this list that overlaps with the AAVR footprint and that demonstrated an enrichment of white variants (a T at position 708) versus black variants (an A at position 708) in the liver. The black variants were underrepresented in the liver (34.8% of the top 256 performing variants), indicating that the T in position 708 of Anc126 enhanced liver targeting versus an A in the same position, which reduced liver targeting. Thus, A708 variants were less efficient in liver uptake compared to T708 variants.

Similarly, data in FIG. 4 indicates that position P5, which corresponds to residue 446 in AAV2 (SEQ ID NO:1), abuts the AAVR footprint and is one of two positions in this list that demonstrated an enrichment of white variants (R446) versus black variants (S446) in the liver. The white variants were underrepresented in the liver (23.6% of the top 512 performing variants), indicating that an R in position 446 of Anc127 enhanced liver targeting versus an S in the same position, which reduced liver targeting.

The data in FIGS. 5A and 5B indicate that position P8 in Anc126 and P7 in Anc127, which both align with residue 471 of AAV2 (SEQ ID NO:1), exhibit only a faint effect with changes at position 471, with viruses having an A471 sequence being slightly more likely to be liver targeted as compared to viruses having an S471 sequence. Since neither the white variants (A471) nor the black variants (S471) strongly determined liver enrichment, not every residue of the predicted AAVR footprint, when conservatively altered, significantly modulates the AAVR-AAV interaction. In addition, an A to S change is a relatively modest change, which also may lead to a less appreciable effect.

Collectively, these data support the discovery that modulation of AAV-AAVR binding alters the uptake by the liver and, by extension, other tissues. Specifically, this analysis indicates that positions 446, 471, and 708 within the AAVR footprint appear to be involved in the bio-distribution of AAV to, or away from, the liver.

### Example 4—Use of an Exemplary Liver Toggle in Non-Human Primates

The AAVAnc80, Anc81, Anc110, and Anc126 variant libraries were injected into the cerebrospinal fluid of two female rhesus macaques (m. mulatta) via an intracisternal route at a dose of 7.75e10 GC/kg to interrogate the impact of the variant sites within the library on CNS, systemic escape, and liver tropism. Animals were sacrificed and tissues were harvested at 7 days following injection. Tissues were subjected to DNA isolation, after which the DNA samples were amplified using PCR with primers flanking the barcode sequence. PCR amplicons underwent a second amplification to incorporate Illumina NGS indexes. These NGS samples were sequenced using Illumina NGS instrumentation MiSeq® or NovaSeq®. Data was subsequently analyzed for each variant to represent enrichment of barcodes in liver as compared to the input injected virus using the barcode counts of the liver versus those in the input viral preparation. A quantitative readout of the liver tissue was obtained, which indicated the extent of liver distribution of each member of the respective library.

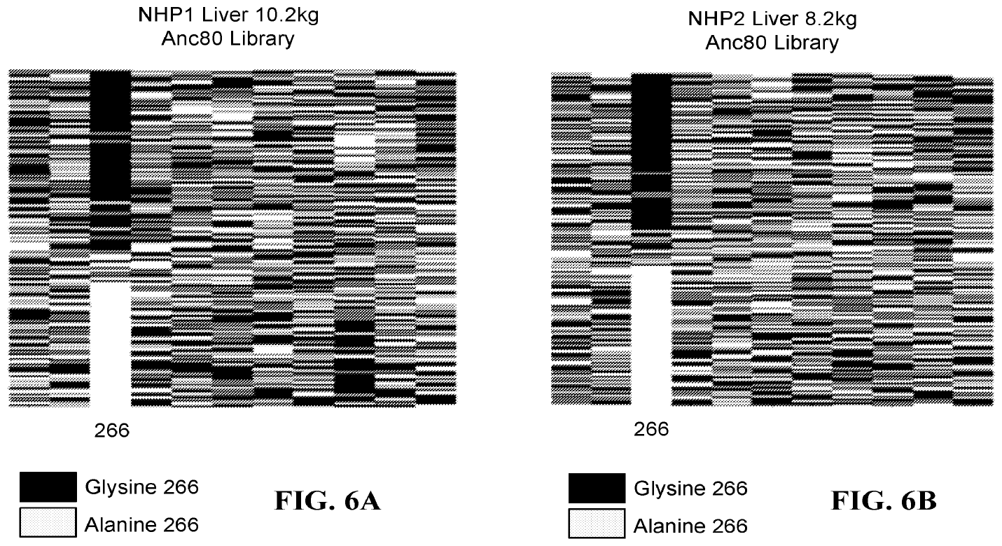

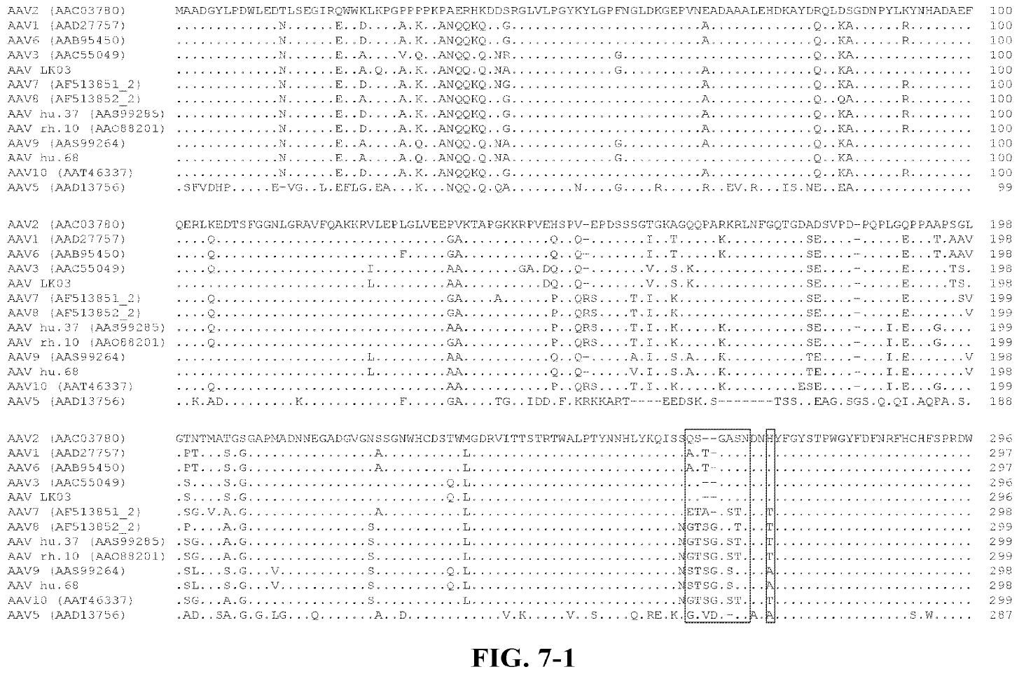

FIGS. 6A and 6B represent the relative performance data in the liver for Anc80 library. The analysis provided here, in the form of graphs referred to as a “fingerprint plots,” illustrates the performance for all members of the Anc80 vector library in rank order from top to bottom. In addition, columns represent the sites of variation within the Anc80 library design. Each site of variation is ambiguous, i.e., has two different residues that can be encoded in that position.

Data in FIGS. 6A and 6B indicate that position P3, which corresponds to residue 266 in AAV2 (SEQ ID NO:1), is the only position in this list that overlaps with the AAVR footprint and that demonstrated an enrichment of black variants (a G at position 266) versus white variants (an A at position 266) in the liver. The white variants were underrepresented in the liver (6.7% of the top 1024 performing variants), indicating that the G in position 266 of Anc80 enhanced liver targeting versus an A in the same position, which reduced liver targeting. Thus, A266 variants were less efficient in liver uptake compared to G266 variants.

Collectively, these data support the discovery that modulation of AAV-AAVR binding alters the uptake by the liver and, by extension, other tissues. Specifically, this analysis indicates that positions 266, 446, 471, and 708 within the AAVR footprint appear to be involved in the bio-distribution of AAV to, or away from, the liver.

### Other Embodiments

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

APPENDIX
X(#) refers to one or more amino acid residues, as indicated, for
the given toggle site.
(VP1 capsid protein of AAV2 and modifications thereof)
SEQ ID NO: 112
MAADGYLPDWLEDTLSEGIRQWWKLKPGPPPPKPAERHKDDSRGLVLPGYKYLGPENGLDKGEPV
NEADAAALEHDKAYDRQLDSGDNPYLKYNHADAEFQERLKEDTSFGGNLGRAVFQAKKRVLEPLG
LVEEPVKTAPGKKRPVEHSPVEPDSSSGTGKAGQQPARKRLNFGQTGDADSVPDPQPLGQPPAAP
SGLGTNTMATGSGAPMADNNEGADGVGNSSGNWHCDSTWMGDRVITTSTRTWALPTYNNHLYKQI
SSX1X2X3X4X5X6DNX7YFGYSTPWGYFDENRFHCHESPRDWQRLINNNWGFRPKRLNFKLFNI
QVKEVTQNDGTTTIANNLTSTVQVFTDSEYQLPYVLGSAHQGCLPPFPADVFMVPQYGYLTLNX8
X9X10X11AVGRSSFYCLEYFPSQMLRTGNNFTFSYTFEDVPFHSSYAHSQSLDRLMNPLIDQYL
YYLX12RTNTPSGTTTQSRLQFSQAGASDIX13DQSRNWLPGPCYRQQRVSKTSADNNNSEYSX1
4X15GATKYHLNGRDSLVNPGPAMASHKX16X17EEKFFPQSGVLIFGKQGSEKTNVDIEKVMIT
DEEEIRTTNPVATEQYGSVSTNLQRGNRX18AATADVNTQGVLPGMVWQDRDVYLQGPIWAKIPH
TDGHFHPSPLMGGFGLKHPPPQILIKNTPVPANPSTTFSAAKFASFITQYSTGQVSVEIEWELQK
ENSKRWNPEIQYTSNYNX19SX20NVDFTVDINGVYSEPRPIGTRYLTRNL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AAV1 and modifications thereof)
SEQ ID NO: 113
AADGYLPDWLEDNLSEGIREWWDLKPGAPKPKANQQKQDDGRGLVLPGYKYLGPENGLDKGEPVN
AADAAALEHDKAYDQQLKAGDNPYLRYNHADAEFQERLQEDTSFGGNLGRAVEQAKKRVLEPLGL
VEEGAKTAPGKKRPVEQSPQEPDSSSGIGKTGQQPAKKRLNFGQTGDSESVPDPQPLGEPPATPA
AVGPTTMASGGGAPMADNNEGADGVGNASGNWHCDSTWLGDRVITTSTRTWALPTYNNHLYKQIS
SX1X2X3X4X5X6DNX7YFGYSTPWGYFDFNRFHCHFSPRDWQRLINNNWGFRPKRLNFKLENIQ
VKEVTTNDGVTTIANNLTSTVQVESDSEYQLPYVLGSAHQGCLPPFPADVEMIPQYGYLTLNX8X
9X10X11AVGRSSFYCLEYFPSQMLRTGNNFTESYTFEEVPFHSSYAHSQSLDRLMNPLIDQYLY
YLX12RTQNQSGSAQNKDLLFSRGSPAGMX13VQPKNWLPGPCYRQQRVSKTKTDNNNSNFTX14
X15GASKYNLNGRESIINPGTAMASHKX16X17EDKFFPMSGVMIFGKESAGASNTALDNVMITD
EEEIKATNPVATERFGTVAVNFQSSSTX18PATGDVHAMGALPGMVWQDRDVYLQGPIWAKIPHT
DGHFHPSPLMGGFGLKNPPPQILIKNTPVPANPPAEFSATKFASFITQYSTGQVSVEIEWELQKE
NSKRWNPEVQYTSNYAX19SX20NVDFTVDNNGLYTEPRPIGTRYLTRPL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AAV6 and modifications thereof)
SEQ ID NO: 114
MAADGYLPDWLEDNLSEGIREWWDLKPGAPKPKANQQKQDDGRGLVLPGYKYLGPENGLDKGEPV
NAADAAALEHDKAYDQQLKAGDNPYLRYNHADAEFQERLQEDTSFGGNLGRAVFQAKKRVLEPFG
LVEEGAKTAPGKKRPVEQSPQEPDSSSGIGKTGQQPAKKRLNFGQTGDSESVPDPQPLGEPPATP
AAVGPTTMASGGGAPMADNNEGADGVGNASGNWHCDSTWLGDRVITTSTRTWALPTYNNHLYKQI
SSX1X2X3X4X5X6DNX7YFGYSTPWGYFDENRFHCHESPRDWQRLINNNWGFRPKRLNEKLENI
QVKEVTTNDGVTTIANNLTSTVQVFSDSEYQLPYVLGSAHQGCLPPFPADVEMIPQYGYLTLNX8
X9X10X11AVGRSSFYCLEYFPSQMLRTGNNFTFSYTFEDVPFHSSYAHSQSLDRLMNPLIDQYL
YYLX12RTQNQSGSAQNKDLLFSRGSPAGMX13VQPKNWLPGPCYRQQRVSKTKTDNNNSNFTX1
4X15GASKYNLNGRESIINPGTAMASHKX16X17KDKFFPMSGVMIFGKESAGASNTALDNVMIT
DEEEIKATNPVATERFGTVAVNLQSSSTX18PATGDVHVMGALPGMVWQDRDVYLQGPIWAKIPH
TDGHFHPSPLMGGFGLKHPPPQILIKNTPVPANPPAEFSATKFASFITQYSTGQVSVEIEWELQK
ENSKRWNPEVQYTSNYAX19SX20NVDFTVDNNGLYTEPRPIGTRYLTRPL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AAV3 and modifications thereof)
SEQ ID NO: 115
MAADGYLPDWLEDNLSEGIREWWALKPGVPQPKANQQHQDNRRGLVLPGYKYLGPGNGLDKGEPV
NEADAAALEHDKAYDQQLKAGDNPYLKYNHADAEFQERLQEDTSEGGNLGRAVFQAKKRILEPLG
LVEEAAKTAPGKKGAVDQSPQEPDSSSGVGKSGKQPARKRLNFGQTGDSESVPDPQPLGEPPAAP
TSLGSNTMASGGGAPMADNNEGADGVGNSSGNWHCDSQWLGDRVITTSTRTWALPTYNNHLYKQI
SSX1X2X3X4X5X6DNX7YFGYSTPWGYFDENRFHCHESPRDWQRLINNNWGFRPKKLSFKLENI
QVRGVTQNDGTTTIANNLTSTVQVFTDSEYQLPYVLGSAHQGCLPPFPADVEMVPQYGYLTLNX8
X9X10X11AVGRSSFYCLEYFPSQMLRTGNNFQFSYTFEDVPFHSSYAHSQSLDRLMNPLIDQYL
YYLX12RTQGTTSGTTNQSRLLFSQAGPQSMX13LQARNWLPGPCYRQQRLSKTANDNNNSNFPX
14X15AASKYHLNGRDSLVNPGPAMASHKX16X17EEKFFPMHGNLIFGKEGTTASNAELDNVMI
TDEEEIRTTNPVATEQYGTVANNLQSSNTX18PTTGTVNHQGALPGMVWQDRDVYLQGPIWAKIP
HTDGHFHPSPLMGGFGLKHPPPQIMIKNTPVPANPPTTFSPAKFASFITQYSTGQVSVEIEWELQ
KENSKRWNPEIQYTSNYNX19SX20NVDFTVDINGVYSEPRPIGTRYLTRNL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AAV LK03 and modifications thereof)
SEQ ID NO: 116
MAADGYLPDWLEDNLSEGIREWWALQPGAPKPKANQQHQDNARGLVLPGYKYLGPGNGLDKGEPV
NAADAAALEHDKAYDQQLKAGDNPYLKYNHADAEFQERLKEDTSFGGNLGRAVEQAKKRLLEPLG
LVEEAAKTAPGKKRPVDQSPQEPDSSSGVGKSGKQPARKRLNFGQTGDSESVPDPQPLGEPPAAP
TSLGSNTMASGGGAPMADNNEGADGVGNSSGNWHCDSQWLGDRVITTSTRTWALPTYNNHLYKQI
SSX1X2X3X4X5X6DNX7YFGYSTPWGYFDENRFHCHFSPRDWQRLINNNWGERPKKLSFKLENI
QVKEVTQNDGTTTIANNLTSTVQVFTDSEYQLPYVLGSAHQGCLPPFPADVEMVPQYGYLTLNX8
X9X10X11AVGRSSFYCLEYFPSQMLRTGNNFQFSYTFEDVPFHSSYAHSQSLDRLMNPLIDQYL
YYLX12RTQGTTSGTTNQSRLLFSQAGPQSMX13LQARNWLPGPCYRQQRLSKTANDNNNSNFPX
14X15AASKYHLNGRDSLVNPGPAMASHKX16X17EEKFFPMHGNLIFGKEGTTASNAELDNVMI
TDEEEIRTTNPVATEQYGTVANNLQSSNTX18PTTRTVNDQGALPGMVWQDRDVYLQGPIWAKIP
HTDGHFHPSPLMGGFGLKHPPPQIMIKNTPVPANPPTTFSPAKFASFITQYSTGQVSVEIEWELQ
KENSKRWNPEIQYTSNYNX19SX20NVDETVDINGVYSEPRPIGTRYLTRPL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AAV7 and modifications thereof)
SEQ ID NO: 117
MAADGYLPDWLEDNLSEGIREWWDLKPGAPKPKANQQKQDNGRGLVLPGYKYLGPENGLDKGEPV
NAADAAALEHDKAYDQQLKAGDNPYLRYNHADAEFQERLQEDTSFGGNLGRAVFQAKKRVLEPLG
LVEEGAKTAPAKKRPVEPSPQRSPDSSTGIGKKGQQPARKRLNFGQTGDSESVPDPQPLGEPPAA
PSSVGSGTVAAGGGAPMADNNEGADGVGNASGNWHCDSTWLGDRVITTSTRTWALPTYNNHLYKQ
ISSX1X2X3X4X5X6DNX7YFGYSTPWGYFDENRFHCHFSPRDWORLINNNWGFRPKKLRFKLEN
IQVKEVTTNDGVTTIANNLTSTIQVFSDSEYQLPYVLGSAHQGCLPPFPADVFMIPQYGYLTLNX
8X9X10X11SVGRSSFYCLEYFPSQMLRTGNNFEFSYSFEDVPFHSSYAHSQSLDRLMNPLIDQY
LYYLX12RTQSNPGGTAGNRELQFYQGGPSTMX13EQAKNWLPGPCFRQQRVSKTLDQNNNSNFA
X14X15GATKYHLNGRNSLVNPGVAMATHKX16X17EDRFFPSSGVLIFGKTGATNKTTLENVLM
TNEEEIRPTNPVATEEYGIVSSNLQAANTX18AQTQVVNNQGALPGMVWQNRDVYLQGPIWAKIP
HTDGNFHPSPLMGGFGLKHPPPQILIKNTPVPANPPEVETPAKFASFITQYSTGQVSVEIEWELQ
KENSKRWNPEIQYTSNFEX19QX20GVDFAVDSQGVYSEPRPIGTRYLTRNL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AAV8 and modifications thereof)
SEQ ID NO: 118
AADGYLPDWLEDNLSEGIREWWALKPGAPKPKANQQKODDGRGLVLPGYKYLGPENGLDKGEPVN
AADAAALEHDKAYDQQLQAGDNPYLRYNHADAEFQERLQEDTSFGGNLGRAVEQAKKRVLEPLGL
VEEGAKTAPGKKRPVEPSPQRSPDSSTGIGKKGQQPARKRLNFGQTGDSESVPDPQPLGEPPAAP
SGVGPNTMAAGGGAPMADNNEGADGVGSSSGNWHCDSTWLGDRVITTSTRTWALPTYNNHLYKQI
SNX1X2X3X4X5X6DNX7YFGYSTPWGYFDENRFHCHFSPRDWORLINNNWGFRPKRLSFKLENI
QVKEVTQNEGTKTIANNLTSTIQVFTDSEYQLPYVLGSAHQGCLPPFPADVEMIPQYGYLTLNX8
X9X10X11AVGRSSFYCLEYFPSQMLRTGNNFQFTYTFEDVPFHSSYAHSQSLDRLMNPLIDQYL
YYLX12RTQTTGGTANTQTLGFSQGGPNTMX13NQAKNWLPGPCYRQQRVSTTTGQNNNSNFAX1
4X15AGTKYHLNGRNSLANPGIAMATHKX16X17EERFFPSNGILIFGKQNAARDNADYSDVMLT
SEEEIKTTNPVATEEYGIVADNLQQQNTX18PQIGTVNSQGALPGMVWQNRDVYLQGPIWAKIPH
TDGNFHPSPLMGGFGLKHPPPQILIKNTPVPADPPTTENQSKLNSFITQYSTGQVSVEIEWELQK
ENSKRWNPEIQYTSNYYX19SX20SVDFAVNTEGVYSEPRPIGTRYLTRNL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AAV hu. 37 and modifications thereof)
SEQ ID NO: 119
MAADGYLPDWLEDNLSEGIREWWDLKPGAPKPKANQQKQDDGRGLVLPGYKYLGPENGLDKGEPV
NAADAAALEHDKAYDQQLKAGDNPYLRYNHADAEFQERLQEDTSEGGNLGRAVFQAKKRVLEPLG
LVEEAAKTAPGKKRPVEPSPQRSPDSSTGIGKKGQQPAKKRLNFGQTGDSESVPDPQPIGEPPAG
PSGLGSGTMAAGGGAPMADNNEGADGVGSSSGNWHCDSTWLGDRVITTSTRTWALPTYNNHLYKQ
ISNX1X2X3X4X5X6DNX7YFGYSTPWGYFDENRFHCHFSPRDWORLINNNWGFRPKRLSFKLEN
IQVKEVTQNEGTKTIANNLTSTIQVFTDSEYQLPYVLGSAHQGCLPPFPADVEMIPQYGYLTLNX
8X9X10X11AVGRSSFYCLEYFPSQMLRTGNNFEFSYTFEDVPFHSSYAHSQSLDRLMNPLIDQY
LYYLX12RTQSTGGTQGTQQLLFSQAGPANMX13AQAKNWLPGPCYRQQRVSTTLSQNNNSNFAX
14X15GATKYHLNGRDSLVNPGVAMATHKX16X17EERFFPSSGVLMFGKQGAGRDNVDYSSVML
TSEEEIKTTNPVATEQYGVVADNLQQTNTX18PIVGNVNSQGALPGMVWQNRDVYLQGPIWAKIP
HTDGNFHPSPLMGGFGLKHPPPQILIKNTPVPADPPTTESQAKLASFITQYSTGQVSVEIEWELQ
KENSKRWNPEIQYTSNYYX19SX20NVDFAVNTEGTYSEPRPIGTRYLTRNL
X1 = Q/A/E/T/G; X2 = G/T/A/V/S; X3 = G/T/A/V; X4-A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AAV rh. 10 and modifications thereof)
SEQ ID NO: 120
MAADGYLPDWLEDNLSEGIREWWDLKPGAPKPKANQQKQDDGRGLVLPGYKYLGPENGLDKGEPV
NAADAAALEHDKAYDQQLKAGDNPYLRYNHADAEFQERLQEDTSFGGNLGRAVFQAKKRVLEPLG
LVEEGAKTAPGKKRPVEPSPQRSPDSSTGIGKKGQQPAKKRLNFGQTGDSESVPDPQPIGEPPAG
PSGLGSGTMAAGGGAPMADNNEGADGVGSSSGNWHCDSTWLGDRVITTSTRTWALPTYNNHLYKQ
ISNX1X2X3X4X5X6DNX7YFGYSTPWGYFDENRFHCHESPRDWQRLINNNWGFRPKRLNFKLEN
IQVKEVTQNEGTKTIANNLTSTIQVFTDSEYQLPYVLGSAHQGCLPPFPADVEMIPQYGYLTLNX
8X9X10X11AVGRSSFYCLEYFPSQMLRTGNNFEFSYQFEDVPFHSSYAHSQSLDRLMNPLIDQY
LYYLX12RTQSTGGTAGTQQLLFSQAGPNNMX13AQAKNWLPGPCYRQQRVSTTLSONNNSNFAX
14X15GATKYHLNGRDSLVNPGVAMATHKX16X17EERFFPSSGVLMFGKQGAGKDNVDYSSVML
TSEEEIKTTNPVATEQYGVVADNLQQQNAX18PIVGAVNSQGALPGMVWQNRDVYLQGPIWAKIP
HTDGNFHPSPLMGGFGLKHPPPQILIKNTPVPADPPTTESQAKLASFITQYSTGQVSVEIEWELQ
KENSKRWNPEIQYTSNYYX19SX20NVDFAVNTDGTYSEPRPIGTRYLTRNL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AA9 and modifications thereof)
SEQ ID NO: 121
MAADGYLPDWLEDNLSEGIREWWALKPGAPQPKANQQHQDNARGLVLPGYKYLGPGNGLDKGEPV
NAADAAALEHDKAYDQQLKAGDNPYLKYNHADAEFQERLKEDTSFGGNLGRAVFQAKKRLLEPLG
LVEEAAKTAPGKKRPVEQSPQEPDSSAGIGKSGAQPAKKRLNFGQTGDTESVPDPQPIGEPPAAP
SGVGSLTMASGGGAPVADNNEGADGVGSSSGNWHCDSQWLGDRVITTSTRTWALPTYNNHLYKQI
SNX1X2X3X4X5X6DNX7YFGYSTPWGYFDENRFHCHFSPRDWQRLINNNWGFRPKRLNEKLENI
QVKEVTDNNGVKTIANNLTSTVQVFTDSDYQLPYVLGSAHEGCLPPFPADVFMIPQYGYLTLNX8
X9X10X11AVGRSSFYCLEYFPSQMLRTGNNFQFSYEFENVPFHSSYAHSQSLDRLMNPLIDQYL
YYLX12KTINGSGQNQQTLKFSVAGPSNMX13VQGRNYIPGPSYRQQRVSTTVTQNNNSEFAX14
X15GASSWALNGRNSLMNPGPAMASHKX16X17EDRFFPLSGSLIFGKQGTGRDNVDADKVMITN
EEEIKTTNPVATESYGQVATNHQSAQAX18AQTGWVQNQGILPGMVWQDRDVYLQGPIWAKIPHT
DGNFHPSPLMGGFGMKHPPPQILIKNTPVPADPPTAFNKDKLNSFITQYSTGQVSVEIEWELQKE
NSKRWNPEIQYTSNYYX19SX20NVEFAVNTEGVYSEPRPIGTRYLTRNL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of hu68 and modifications thereof)
SEQ ID NO: 122
MAADGYLPDWLEDNLSEGIREWWALKPGAPQPKANQQHQDNARGLVLPGYKYLGPGNGLDKGEPV
NEADAAALEHDKAYDQQLKAGDNPYLKYNHADAEFQERLKEDTSFGGNLGRAVFQAKKRLLEPLG
LVEEAAKTAPGKKRPVEQSPQEPDSSVGIGKSGAQPAKKRLNFGQTGDTESVPDPQPIGEPPAAP
SGVGSLTMASGGGAPVADNNEGADGVGSSSGNWHCDSQWLGDRVITTSTRTWALPTYNNHLYKQI
SNX1X2X3X4X5X6DNX7YFGYSTPWGYFDENRFHCHFSPRDWQRLINNNWGFRPKRLNFKLENI
QVKEVTDNNGVKTIANNLTSTVQVFTDSDYQLPYVLGSAHEGCLPPFPADVEMIPQYGYLTLNX8
X9X10X11AVGRSSFYCLEYFPSQMLRTGNNFQFSYEFENVPFHSSYAHSQSLDRLMNPLIDQYL
YYLX12KTINGSGQNQQTLKFSVAGPSNMX13VQGRNYIPGPSYRQQRVSTTVTQNNNSEFAX14
X15GASSWALNGRNSLMNPGPAMASHKX16X17EDRFFPLSGSLIFGKQGTGRDNVDADKVMITN
EEEIKTTNPVATESYGQVATNHQSAQAX18AQTGWVQNQGILPGMVWQDRDVYLQGPIWAKIPHT
DGNFHPSPLMGGFGMKHPPPQILIKNTPVPADPPTAFNKDKLNSFITQYSTGQVSVEIEWELQKE
NSKRWNPEIQYTSNYYX19SX2ONVEFAVNTEGVYSEPRPIGTRYLTRNL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AAV10 and modifications thereof)
SEQ ID NO: 123
MAADGYLPDWLEDNLSEGIREWWDLKPGAPKPKANQQKQDDGRGLVLPGYKYLGPENGLDKGEPV
NAADAAALEHDKAYDQQLKAGDNPYLRYNHADAEFQERLQEDTSEGGNLGRAVEQAKKRVLEPLG
LVEEAAKTAPGKKRPVEPSPQRSPDSSTGIGKKGQQPAKKRLNFGQTGESESVPDPQPIGEPPAG
PSGLGSGTMAAGGGAPMADNNEGADGVGSSSGNWHCDSTWLGDRVITTSTRTWALPTYNNHLYKQ
ISNX1X2X3X4X5X6DNX7YFGYSTPWGYFDENRFHCHESPRDWQRLINNNWGFRPKRLSFKLEN
IQVKEVTQNEGTKTIANNLTSTIQVFTDSEYQLPYVLGSAHQGCLPPFPADVEMIPQYGYLTLNX
8X9X10X11AVGRSSFYCLEYFPSQMLRTGNNFEFSYTFEDVPFHSSYAHSQSLDRLMNPLIDQY
LYYLX12RTQSTGGTQGTQQLLFSQAGPANMX13AQAKNWLPGPCYRQQRVSTTLSQNNNSNFAX
14X15GATKYHLNGRDSLVNPGVAMATHKX16X17EERFFPSSGVLMFGKQGAGRDNVDYSSVML
TSEEEIKTTNPVATEQYGVVADNLQQANTX18PIVGNVNSQGALPGMVWQNRDVYLQGPIWAKIP
HTDGNFHPSPLMGGFGLKHPPPQILIKNTPVPADPPTTESQAKLASFITQYSTGQVSVEIEWELQ
KENSKRWNPEIQYTSNYYX19SX20NVDFAVNTEGTYSEPRPIGTRYLTRNL
X1 = Q/A/E/T/G; X2 = G/T/A/V/S; X3 = G/T/A/V; X4-A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid protein of AA5 and modifications thereof)
SEQ ID NO: 124
MSFVDHPPDWLEEVGEGLREFLGLEAGPPKPKPNQQHQDQARGLVLPGYNYLGPGNGLDRGEPVN
RADEVAREHDISYNEQLEAGDNPYLKYNHADAEFQEKLADDTSFGGNLGKAVFQAKKRVLEPFGL
VEEGAKTAPTGKRIDDHFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNNHQYREIKSX1X2X3X4
X5X6ANX7YFGYSTPWGYFDENRFHSHWSPRDWQRLINNYWGERPRSLRVKIFNIQVKEVTVQDS
TTTIANNLTSTVQVFTDDDYQLPYVVGNGTEGCLPAFPPQVFTLPQYGYATLNRDX8X9X10X11
PTERSSFFCLEYFPSKMLRTGNNFEFTYNFEEVPFHSSFAPSQNLEKLANPLVDQYLYRFX12ST
NNTGGVQFNKNLAGRYX13NTYKNWFPGPMGRTQGWNLGSGVNRASVSAX14X15TTNRMELEGA
SYQVPPQPNGMTNNX16X17GSNTYALENTMIFNSQPANPGTTATYLEGNMLITSESETQPVNRV
AYNVGGQMATNNQSSTTX18PATGTYNLQEIVPGSVWMERDVYLQGPIWAKIPETGAHFHPSPAM
GGFGLKHPPPMMLIKNTPVPGNITSFSDVPVSSFITQYSTGQVTVEMEWELKKENSKRWNPEIQY
TNNYNX19PX20FVDFAPDSTGEYRTTRPIGTRYLTRPL
X1 = Q/A/E/T/G; X2 = G/T/A/V/S; X3 = G/T/A/V; X4 = A/G/D; X5 = S/T; X6 = N;
X7 = H/T/A; X8 = N/D; X9 = G/T; X10 = S/E; X11 = Q/N; X12 = S/N/A/V/R;
X13 = R/S/A; X14 = W/F; X15 = T/P/A; X16 = D/E/L; X17 = D/G/Q; X18 = Q/D/A/G;
X19 = K/D; X20 = V/A/T/N/Q
(VP1 capsid proteins of AAV2 and modifications thereof)
SEQ ID NO: 125
MAADGYLPDWLEDTLSEGIRQWWKLKPGPPPPKPAERHKDDSRGLVLPGYKYLGPENGLDKGEPV
NEADAAALEHDKAYDRQLDSGDNPYLKYNHADAEFQERLKEDTSFGGNLGRAVFQAKKRVLEPLG
LVEEPVKTAPGKKRPVEHSPVEPDSSSGTGKAGQQPARKRLNFGQTGDADSVPDPQPLGQPPAAP
SGLGTNTMATGSGAPMADNNEGADGVGNSSGNWHCDSTWMGDRVITTSTRTWALPTYNNHLYKQI
SSQSGASNDNHYFGYSTPWGYFDFNRFHCHFSPRDWORLINNNWGFRPKRLNFKLENIQVKEVTQ
NDGTTTIANNLTSTVQVFTDSEYQLPYVLGSAHQGCLPPFPADVFMVPQYGYLTLNNGSQAVGRS
SFYCLEYFPSQMLRTGNNFTFSYTFEDVPFHSSYAHSQSLDRLMNPLIDQYLYYLX1RTNTPSGT
TTQSRLQFSQAGASDIX2DQSRNWLPGPCYRQQRVSKTSADNNNSEYSWTGATKYHLNGRDSLVN
PGPAMASHKDDEEKFFPQSGVLIFGKQGSEKTNVDIEKVMITDEEEIRTTNPVATEQYGSVSTNL
QRGNRQAATADVNTQGVLPGMVWQDRDVYLQGPIWAKIPHTDGHFHPSPLMGGFGLKHPPPQILI
KNTPVPANPSTTESAAKFASFITQYSTGQVSVEIEWELQKENSKRWNPEIQYTSNYNKSX3NVDF
TVDTNGVYSEPRPIGTRYLTRNL
X1 = R/S; X2 = A/R/S; X3 = T/V/A
(VP1 capsid proteins of AAV1 and modifications thereof)
SEQ ID NO: 126
AADGYLPDWLEDNLSEGIREWWDLKPGAPKPKANQQKQDDGRGLVLPGYKYLGPENGLDKGEPVN
AADAAALEHDKAYDQQLKAGDNPYLRYNHADAEFQERLQEDTSFGGNLGRAVEQAKKRVLEPLGL
VEEGAKTAPGKKRPVEQSPQEPDSSSGIGKTGQQPAKKRLNFGQTGDSESVPDPOPLGEPPATPA
AVGPTTMASGGGAPMADNNEGADGVGNASGNWHCDSTWLGDRVITTSTRTWALPTYNNHLYKQIS
SASTGASNDNHYFGYSTPWGYFDENRFHCHESPRDWQRLINNNWGFRPKRLNEKLFNIQVKEVTT
NDGVTTIANNLTSTVQVESDSEYQLPYVLGSAHQGCLPPFPADVEMIPQYGYLTLNNGSQAVGRS
SFYCLEYFPSQMLRTGNNFTFSYTFEEVPFHSSYAHSQSLDRLMNPLIDQYLYYLX1RTQNQSGS
AQNKDLLFSRGSPAGMX2VQPKNWLPGPCYRQQRVSKTKTDNNNSNFTWTGASKYNLNGRESIIN
PGTAMASHKDDEDKFFPMSGVMIFGKESAGASNTALDNVMITDEEEIKATNPVATERFGTVAVNF
QSSSTDPATGDVHAMGALPGMVWQDRDVYLQGPIWAKIPHTDGHFHPSPLMGGFGLKNPPPQILI
KNTPVPANPPAEFSATKFASFITQYSTGQVSVEIEWELQKENSKRWNPEVQYTSNYAKSX3NVDF
TVDNNGLYTEPRPIGTRYLTRPL
X1 = R/S; X2 = A/R/S; X3 = T/V/A
(VP1 capsid proteins of AAV6 and modifications thereof)
SEQ ID NO: 127
MAADGYLPDWLEDNLSEGIREWWDLKPGAPKPKANQQKQDDGRGLVLPGYKYLGPENGLDKGEPV
NAADAAALEHDKAYDQQLKAGDNPYLRYNHADAEFQERLQEDTSFGGNLGRAVFQAKKRVLEPFG
LVEEGAKTAPGKKRPVEQSPQEPDSSSGIGKTGQQPAKKRLNFGQTGDSESVPDPQPLGEPPATP
AAVGPTTMASGGGAPMADNNEGADGVGNASGNWHCDSTWLGDRVITTSTRTWALPTYNNHLYKQI
SSASTGASNDNHYFGYSTPWGYEDENRFHCHESPRDWQRLINNNWGFRPKRLNFKLFNIQVKEVT
TNDGVTTIANNLTSTVQVESDSEYQLPYVLGSAHQGCLPPFPADVEMIPQYGYLTLNNGSQAVGR
SSFYCLEYFPSQMLRTGNNFTFSYTFEDVPFHSSYAHSQSLDRLMNPLIDQYLYYLX1RTQNQSG
SAQNKDLLFSRGSPAGMX2VQPKNWLPGPCYRQQRVSKTKTDNNNSNFTWTGASKYNLNGRESII
NPGTAMASHKDDKDKFFPMSGVMIFGKESAGASNTALDNVMITDEEEIKATNPVATERFGTVAVN
LQSSSTDPATGDVHVMGALPGMVWQDRDVYLQGPIWAKIPHTDGHFHPSPLMGGFGLKHPPPQIL
IKNTPVPANPPAEFSATKFASFITQYSTGQVSVEIEWELQKENSKRWNPEVQYTSNYAKSX3NVD
FTVDNNGLYTEPRPIGTRYLTRPL
X1 = R/S; X2 = A/R/S; X3 = T/V/A
(VP1 capsid proteins of AAV3 and modifications thereof)
SEQ ID NO: 128
MAADGYLPDWLEDNLSEGIREWWALKPGVPQPKANQQHQDNRRGLVLPGYKYLGPGNGLDKGEPV
NEADAAALEHDKAYDQQLKAGDNPYLKYNHADAEFQERLQEDTSFGGNLGRAVEQAKKRILEPLG
LVEEAAKTAPGKKGAVDQSPQEPDSSSGVGKSGKQPARKRLNFGQTGDSESVPDPQPLGEPPAAP
TSLGSNTMASGGGAPMADNNEGADGVGNSSGNWHCDSQWLGDRVITTSTRTWALPTYNNHLYKQI
SSQSGASNDNHYFGYSTPWGYFDENRFHCHESPRDWQRLINNNWGFRPKKLSFKLENIQVRGVTQ
NDGTTTIANNLTSTVQVFTDSEYQLPYVLGSAHQGCLPPFPADVEMVPQYGYLTLNNGSQAVGRS
SFYCLEYFPSQMLRTGNNFQFSYTFEDVPFHSSYAHSQSLDRLMNPLIDQYLYYLX1RTQGTTSG
TTNQSRLLFSQAGPQSMX2LQARNWLPGPCYRQQRLSKTANDNNNSNFPWTAASKYHLNGRDSLV
NPGPAMASHKDDEEKFFPMHGNLIFGKEGTTASNAELDNVMITDEEEIRTTNPVATEQYGTVANN
LQSSNTAPTTGTVNHQGALPGMVWQDRDVYLQGPIWAKIPHTDGHFHPSPLMGGFGLKHPPPQIM
IKNTPVPANPPTTFSPAKFASFITQYSTGQVSVEIEWELQKENSKRWNPEIQYTSNYNKSX3NVD
FTVDTNGVYSEPRPIGTRYLTRNL
X1 = R/S; X2 = A/R/S; X3 = T/V/A
(VP1 capsid proteins of AAV LK03 and modifications thereof)
SEQ ID NO: 129
MAADGYLPDWLEDNLSEGIREWWALQPGAPKPKANQQHQDNARGLVLPGYKYLGPGNGLDKGEPV
NAADAAALEHDKAYDQQLKAGDNPYLKYNHADAEFQERLKEDTSFGGNLGRAVFQAKKRLLEPLG
LVEEAAKTAPGKKRPVDQSPQEPDSSSGVGKSGKQPARKRLNFGQTGDSESVPDPQPLGEPPAAP
TSLGSNTMASGGGAPMADNNEGADGVGNSSGNWHCDSQWLGDRVITTSTRTWALPTYNNHLYKQI
SSQSGASNDNHYFGYSTPWGYFDENRFHCHESPRDWQRLINNNWGFRPKKLSFKLENIQVKEVTQ
NDGTTTIANNLTSTVQVFTDSEYQLPYVLGSAHQGCLPPFPADVEMVPQYGYLTLNNGSQAVGRS
SFYCLEYFPSQMLRTGNNFQFSYTFEDVPFHSSYAHSQSLDRLMNPLIDQYLYYLX1RTQGTTSG
TTNQSRLLFSQAGPQSMX2LQARNWLPGPCYRQQRLSKTANDNNNSNFPWTAASKYHLNGRDSLV
NPGPAMASHKDDEEKFFPMHGNLIFGKEGTTASNAELDNVMITDEEEIRTTNPVATEQYGTVANN
LQSSNTAPTTRTVNDQGALPGMVWQDRDVYLQGPIWAKIPHTDGHFHPSPLMGGFGLKHPPPQIM
IKNTPVPANPPTTFSPAKFASFITQYSTGQVSVEIEWELQKENSKRWNPEIQYTSNYNKSX3NVD
FTVDTNGVYSEPRPIGTRYLTRPL
X1 = R/S; X2 = A/R/S; X3 = T/V/A
(VP1 capsid protein of AAV7 and modifications thereof)
SEQ ID NO: 130
MAADGYLPDWLEDNLSEGIREWWDLKPGAPKPKANQQKQDNGRGLVLPGYKYLGPENGLDKGEPV
NAADAAALEHDKAYDQQLKAGDNPYLRYNHADAEFQERLQEDTSEGGNLGRAVEQAKKRVLEPLG
LVEEGAKTAPAKKRPVEPSPQRSPDSSTGIGKKGQQPARKRLNFGQTGDSESVPDPQPLGEPPAA
PSSVGSGTVAAGGGAPMADNNEGADGVGNASGNWHCDSTWLGDRVITTSTRTWALPTYNNHLYKQ
ISSETAGSTNDNTYFGYSTPWGYFDFNRFHCHESPRDWQRLINNNWGERPKKLRFKLENIQVKEV
TTNDGVTTIANNLTSTIQVFSDSEYQLPYVLGSAHQGCLPPFPADVEMIPQYGYLTLNNGSQSVG
RSSFYCLEYFPSQMLRTGNNFEFSYSFEDVPFHSSYAHSQSLDRLMNPLIDQYLYYLX1RTQSNP
GGTAGNRELQFYQGGPSTMX2EQAKNWLPGPCFRQQRVSKTLDQNNNSNFAWTGATKYHLNGRNS
LVNPGVAMATHKDDEDRFFPSSGVLIFGKTGATNKTTLENVLMTNEEEIRPTNPVATEEYGIVSS
NLQAANTAAQTQVVNNQGALPGMVWQNRDVYLQGPIWAKIPHTDGNFHPSPLMGGFGLKHPPPQI
LIKNTPVPANPPEVFTPAKFASFITQYSTGQVSVEIEWELQKENSKRWNPEIQYTSNFEKQ
X3GVDFAVDSQGVYSEPRPIGTRYLTRNL
X1 = R/S; X2 = A/R/S; X3 = T/V/A
(VP1 capsid protein of AAV8 and modifications thereof)
SEQ ID NO: 131
AADGYLPDWLEDNLSEGIREWWALKPGAPKPKANQQKQDDGRGLVLPGYKYLGPENGLDKGEPVN
AADAAALEHDKAYDQQLQAGDNPYLRYNHADAEFQERLQEDTSFGGNLGRAVEQAKKRVLEPLGL
VEEGAKTAPGKKRPVEPSPQRSPDSSTGIGKKGQQPARKRLNFGQTGDSESVPDPQPLGEPPAAP
SGVGPNTMAAGGGAPMADNNEGADGVGSSSGNWHCDSTWLGDRVITTSTRTWALPTYNNHLYKQI
SNGTSGGATNDNTYFGYSTPWGYFDENRFHCHFSPRDWORLINNNWGFRPKRLSFKLFNIQVKEV
TQNEGTKTIANNLTSTIQVFTDSEYQLPYVLGSAHQGCLPPFPADVEMIPQYGYLTLNNGSQAVG
RSSFYCLEYFPSQMLRTGNNFQFTYTFEDVPFHSSYAHSQSLDRLMNPLIDQYLYYLX1RTQTTG
GTANTQTLGFSQGGPNTMX2NQAKNWLPGPCYRQQRVSTTTGQNNNSNFAWTAGTKYHLNGRNSL
ANPGIAMATHKDDEERFFPSNGILIFGKQNAARDNADYSDVMLTSEEEIKTTNPVATEEYGIVAD
NLQQQNTAPQIGTVNSQGALPGMVWQNRDVYLQGPIWAKIPHTDGNFHPSPLMGGFGLKHPPPQI
LIKNTPVPADPPTTENQSKLNSFITQYSTGQVSVEIEWELQKENSKRWNPEIQYTSNYYKSX3SV
DFAVNTEGVYSEPRPIGTRYLTRNL
X1 = R/S; X2 = A/R/S; X3 = T/V/A
(VP1 capsid protein of AAV hu. 37 and modifications thereof)
SEQ ID NO: 132
MAADGYLPDWLEDNLSEGIREWWDLKPGAPKPKANQQKQDDGRGLVLPGYKYLGPENGLDKGEPV
NAADAAALEHDKAYDQQLKAGDNPYLRYNHADAEFQERLQEDTSEGGNLGRAVEQAKKRVLEPLG
LVEEAAKTAPGKKRPVEPSPQRSPDSSTGIGKKGQQPAKKRLNFGQTGDSESVPDPQPIGEPPAG
PSGLGSGTMAAGGGAPMADNNEGADGVGSSSGNWHCDSTWLGDRVITTSTRTWALPTYNNHLYKQ
ISNGTSGGSTNDNTYFGYSTPWGYFDFNRFHCHFSPRDWQRLINNNWGFRPKRLSFKLFNIQVKE
VTQNEGTKTIANNLTSTIQVFTDSEYQLPYVLGSAHQGCLPPFPADVEMIPQYGYLTLNNGSQAV
GRSSFYCLEYFPSQMLRTGNNFEFSYTFEDVPFHSSYAHSQSLDRLMNPLIDQYLYYLX1RTQST
GGTQGTQQLLFSQAGPANMX2AQAKNWLPGPCYRQQRVSTTLSQNNNSNFAWTGATKYHLNGRDS
LVNPGVAMATHKDDEERFFPSSGVLMFGKQGAGRDNVDYSSVMLTSEEEIKTTNPVATEQYGVVA
DNLQQTNTGPIVGNVNSQGALPGMVWQNRDVYLQGPIWAKIPHTDGNFHPSPLMGGFGLKHPPPQ
ILIKNTPVPADPPTTESQAKLASFITQYSTGQVSVEIEWELQKENSKRWNPEIQYTSNYYKSX3N
VDFAVNTEGTYSEPRPIGTRYLTRNL
X1 = R/S; X2 = A/R/S; X3 = T/V/A
(VP1 capsid protein of AAV rh.10 and modifications thereof)
SEQ ID NO: 133
MAADGYLPDWLEDNLSEGIREWWDLKPGAPKPKANQQKQDDGRGLVLPGYKYLGPENGLDKGEPV
NAADAAALEHDKAYDQQLKAGDNPYLRYNHADAEFQERLQEDTSFGGNLGRAVFQAKKRVLEPLG
LVEEGAKTAPGKKRPVEPSPQRSPDSSTGIGKKGQQPAKKRLNFGQTGDSESVPDPQPIGEPPAG
PSGLGSGTMAAGGGAPMADNNEGADGVGSSSGNWHCDSTWLGDRVITTSTRTWALPTYNNHLYKQ
ISNGTSGGSTNDNTYFGYSTPWGYFDENRFHCHESPRDWQRLINNNWGFRPKRLNFKLENIQVKE
VTQNEGTKTIANNLTSTIQVFTDSEYQLPYVLGSAHQGCLPPFPADVEMIPQYGYLTLNNGSQAV
GRSSFYCLEYFPSQMLRTGNNFEFSYQFEDVPFHSSYAHSQSLDRLMNPLIDQYLYYLX1RTQST
GGTAGTQQLLFSQAGPNNMX2AQAKNWLPGPCYRQQRVSTTLSQNNNSNFAWTGATKYHLNGRDS
LVNPGVAMATHKDDEERFFPSSGVLMFGKQGAGKDNVDYSSVMLTSEEEIKTTNPVATEQYGVVA
DNLQQQNAAPIVGAVNSQGALPGMVWQNRDVYLQGPIWAKIPHTDGNFHPSPLMGGFGLKHPPPQ
ILIKNTPVPADPPTTESQAKLASFITQYSTGQVSVEIEWELQKENSKRWNPEIQYTSNYYKSX3N
VDFAVNTDGTYSEPRPIGTRYLTRNL
X1 = R/S; X2 = A/R/S; X3 = T/V/A
(VP1 capsid protein of AA9 and modifications thereof)
SEQ ID NO: 134
MAADGYLPDWLEDNLSEGIREWWALKPGAPQPKANQQHQDNARGLVLPGYKYLGPGNGLDKGEPV
NAADAAALEHDKAYDQQLKAGDNPYLKYNHADAEFQERLKEDTSFGGNLGRAVFQAKKRLLEPLG
LVEEAAKTAPGKKRPVEQSPQEPDSSAGIGKSGAQPAKKRLNFGQTGDTESVPDPQPIGEPPAAP
SGVGSLTMASGGGAPVADNNEGADGVGSSSGNWHCDSQWLGDRVITTSTRTWALPTYNNHLYKQI
SNSTSGGSSNDNAYFGYSTPWGYFDFNRFHCHFSPRDWORLINNNWGFRPKRLNFKLENIQVKEV
TDNNGVKTIANNLTSTVQVFTDSDYQLPYVLGSAHEGCLPPFPADVEMIPQYGYLTLNDGSQAVG
RSSFYCLEYFPSQMLRTGNNFQFSYEFENVPFHSSYAHSQSLDRLMNPLIDQYLYYLX1KTINGS
GQNQQTLKFSVAGPSNMX2VQGRNYIPGPSYRQQRVSTTVTQNNNSEFAWPGASSWALNGRNSLM
NPGPAMASHKEGEDRFFPLSGSLIFGKQGTGRDNVDADKVMITNEEEIKTTNPVATESYGQVATN
HQSAQAQAQTGWVQNQGILPGMVWQDRDVYLQGPIWAKIPHTDGNFHPSPLMGGFGMKHPPPQIL
IKNTPVPADPPTAFNKDKLNSFITQYSTGQVSVEIEWELQKENSKRWNPEIQYTSNYYKSX3NVE
FAVNTEGVYSEPRPIGTRYLTRNL
X1 = R/S; X2 = A/R/S; X3 = T/V/A
(VP1 capsid protein of hu68 and modifications thereof)
SEQ ID NO: 135
MAADGYLPDWLEDNLSEGIREWWALKPGAPQPKANQQHQDNARGLVLPGYKYLGPGNGLDKGEPV
NEADAAALEHDKAYDQQLKAGDNPYLKYNHADAEFQERLKEDTSFGGNLGRAVEQAKKRLLEPLG
LVEEAAKTAPGKKRPVEQSPQEPDSSVGIGKSGAQPAKKRLNFGQTGDTESVPDPQPIGEPPAAP
SGVGSLTMASGGGAPVADNNEGADGVGSSSGNWHCDSQWLGDRVITTSTRTWALPTYNNHLYKQI
SNSTSGGSSNDNAYFGYSTPWGYFDENRFHCHESPRDWORLINNNWGFRPKRLNEKLENIQVKEV
TDNNGVKTIANNLTSTVQVFTDSDYQLPYVLGSAHEGCLPPFPADVEMIPQYGYLTLNDGSQAVG
RSSFYCLEYFPSQMLRTGNNFQFSYEFENVPFHSSYAHSQSLDRLMNPLIDQYLYYLX1KTINGS
GQNQQTLKFSVAGPSNMX2VQGRNYIPGPSYRQQRVSTTVTQNNNSEFAWPGASSWALNGRNSLM
NPGPAMASHKEGEDRFFPLSGSLIFGKQGTGRDNVDADKVMITNEEEIKTTNPVATESYGQVATN
HQSAQAQAQTGWVQNQGILPGMVWQDRDVYLQGPIWAKIPHTDGNFHPSPLMGGFGMKHPPPQIL
IKNTPVPADPPTAFNKDKLNSFITQYSTGQVSVEIEWELQKENSKRWNPEIQYTSNYYKSX3NVE
FAVNTEGVYSEPRPIGTRYLTRNL
X1 = R/S; X2 = A/R/S; X3 = T/V/A
(VP1 capsid protein of AAV10 and modifications thereof)
SEQ ID NO: 136
MAADGYLPDWLEDNLSEGIREWWDLKPGAPKPKANQQKQDDGRGLVLPGYKYLGPENGLDKGEPV
NAADAAALEHDKAYDQQLKAGDNPYLRYNHADAEFQERLQEDTSEGGNLGRAVEQAKKRVLEPLG
LVEEAAKTAPGKKRPVEPSPQRSPDSSTGIGKKGQQPAKKRLNFGQTGESESVPDPQPIGEPPAG
PSGLGSGTMAAGGGAPMADNNEGADGVGSSSGNWHCDSTWLGDRVITTSTRTWALPTYNNHLYKQ
ISNGTSGGSTNDNTYFGYSTPWGYFDENRFHCHESPRDWQRLINNNWGFRPKRLSFKLENIQVKE
VTQNEGTKTIANNLTSTIQVFTDSEYQLPYVLGSAHQGCLPPFPADVEMIPQYGYLTLNNGSQAV
GRSSFYCLEYFPSQMLRTGNNFEFSYTFEDVPFHSSYAHSQSLDRLMNPLIDQYLYYLX1RTQST
GGTQGTQQLLFSQAGPANMX2AQAKNWLPGPCYRQQRVSTTLSQNNNSNFAWTGATKYHLNGRDS
LVNPGVAMATHKDDEERFFPSSGVLMFGKOGAGRDNVDYSSVMLTSEEEIKTTNPVATEQYGVVA
DNLQQANTGPIVGNVNSQGALPGMVWQNRDVYLQGPIWAKIPHTDGNFHPSPLMGGFGLKHPPPQ
ILIKNTPVPADPPTTESQAKLASFITQYSTGQVSVEIEWELQKENSKRWNPEIQYTSNYYKSX3N
VDFAVNTEGTYSEPRPIGTRYLTRNL
X1 = R/S; X2 = A/R/S; X3 = T/V/A
(VP1 capsid protein of AA5 and modifications thereof)
SEQ ID NO: 137
MSFVDHPPDWLEEVGEGLREFLGLEAGPPKPKPNQQHQDQARGLVLPGYNYLGPGNGLDRGEPVN
RADEVAREHDISYNEQLEAGDNPYLKYNHADAEFQEKLADDTSFGGNLGKAVFQAKKRVLEPFGL
VEEGAKTAPTGKRIDDHFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNNHQYREIKSGSVDGSNA
NAYFGYSTPWGYFDFNRFHSHWSPRDWQRLINNYWGERPRSLRVKIFNIQVKEVTVQDSTTTIAN
NLTSTVQVFTDDDYQLPYVVGNGTEGCLPAFPPQVFTLPQYGYATLNRDNTENPTERSSFFCLEY
FPSKMLRTGNNFEFTYNFEEVPFHSSFAPSQNLFKLANPLVDQYLYRFX1STNNTGGVQENKNLA
GRYX2NTYKNWFPGPMGRTQGWNLGSGVNRASVSAFATTNRMELEGASYQVPPQPNGMTNNLQGS
NTYALENTMIENSQPANPGTTATYLEGNMLITSESETQPVNRVAYNVGGQMATNNQSSTTAPATG
TYNLQEIVPGSVWMERDVYLQGPIWAKIPETGAHFHPSPAMGGFGLKHPPPMMLIKNTPVPGNIT
SFSDVPVSSFITQYSTGQVTVEMEWELKKENSKRWNPEIQYTNNYNDPX3FVDFAPDSTGEYRTT
RPIGTRYLTRPL
X1 = R/S; X2 = A/R/S; X3 = T/V/A
(Anc80 scaffold)
SEQ ID NO: 138
MAADGYLPDWLEDNLSEGIREWWDLKPGAPKPKANQQKQDDGRGLVLPGYKYLGPENGLD
KGEPVNAADAAALEHDKAYDQQLKAGDNPYLRYNHADAEFQERLQEDTSEGGNLGRAVEQ
AKKRVLEPLGLVEEGAKTAPGKKRPVEQSPQEPDSSSGIGKKGQQPAXKRLNFGQTGDSE
SVPDPQPLGEPPAAPSGVGSNTMAXGGGAPMADNNEGADGVGNASGNWHCDSTWLGDRVI
TTSTRTWALPTYNNHLYKQISSQSGXSTNDNTYFGYSTPWGYFDENRFHCHESPRDWORL
INNNWGFRPKXLNFKLFNIQVKEVTTNDGTTTIANNLTSTVQVFTDSEYQLPYVLGSAHQ
GCLPPFPADVEMIPQYGYLTLNNGSQAVGRSSFYCLEYFPSQMLRTGNNFXFSYTFEDVP
FHSSYAHSQSLDRLMNPLIDQYLYYLSRTQTTSGTAGNRXLQFSQAGPSSMANQAKNWLP
GPCYRQQRVSKTXNQNNNSNFAWTGATKYHLNGRDSLVNPGPAMATHKDDEDKFFPMSGV
LIFGKQGAGNSNVDLDNVMITXEEEIKTTNPVATEXYGTVATNLQSXNTAPATGTVNSQG
ALPGMVWQXRDVYLQGPIWAKIPHTDGHFHPSPLMGGFGLKHPPPQILIKNTPVPANPPT
TFSPAKFASFITQYSTGQVSVEIEWELQKENSKRWNPEIQYTSNYNKSTNVDFAVDINGV
YSEPRPIGTRYLTRNL
<222> (168) . . . (168)
<223> Lys or Arg
<222> (205) . . . (205)
<223> Ala or Ser
<222> (266) . . . (266)
<223> Ala or Gly
<222> (311) . . . (311)
<223> Arg or Lys
<222> (411) . . . (411)
<223> Glu or Gln
<222> (460) . . . (460)
<223> Thr or Glu
<222> (493) . . . (493)
<223> Ala or Thr
<222> (562) . . . (562)
<223> Ser or Asn
<222> (576) . . . (576)
<223> Gln or Glu
<222> (587) . . . (587)
<223> Ser or Ala
<222> (609) . . . (609)
<223> Asn or Asp
(Anc81 scaffold)
SEQ ID NO: 139
MAADGYLPDWLEDNLSEGIREWWDLKPGAPKPKANQQKQDDGRGLVLPGYKYLGPENGLD
KGEPVNAADAAALEHDKAYDQQLKAGDNPYLRYNHADAEFQERLQEDTSFGGNLGRAVEQ
AKKRVLEPLGLVEEGAKTAPGKKRPVEQSPQEPDSSXGIGKKGQQPAXKRLNFGQTGDSE
SVPDPQPLGEPPAAPSGVGSNTMAAGGGAPMADNNEGADGVGNASGNWHCDSTWLGDRVI
TTSTRTWALPTYNNHLYKQISXXQSGGSTNDNTYFGYSTPWGYFDENRFHCHESPRDWQR
LINNNWGFRPKXLNFKLFNIQVKEVTTNDGTTTIANNLTSTVQVFTDSEYQLPYVLGSAH
QGCLPPFPADVFMIPQYGYLTLNNGSQAVGRSSFYCLEYFPSQMLRTGNNEXFSYTFEDV
PFHSSYAHSQSLDRLMNPLIDQYLYYLSRTQTTGGTAGNXXLQFSQAGPSSMANQAKNWL
PGPCYRQQRVSKTTNQNNNSNFAWTGATKYHLNGRDSLVNPGVAMATHKDDEDREFPSSG
VLIFGKQGAGNXNVDXXNVMITXEEEIKTTNPVATEEYGXVATNLQSXNTAPQTGTVNSQ
GALPGMVWQNRDVYLQGPIWAKIPHTDGNFHPSPLMGGFGLKHPPPQILIKNTPVPANPP
TTFXPAKFASFITQYSTGQVSVEIEWELQKENSKRWNPEIQYTSNYNKSTNVDFAVDTEG
VYSEPRPIGTRYLTRNL
<222> (157) . . . (157)
<223> Thr or Ser
<222> (168) . . . (168)
<223> Lys or Arg
<222> (262) . . . (262)
<223> Asn or Ser
<222> (263) . . . (263)
<223> Ser or His
<222> (312) . . . (312)
<223> Arg or Lys
<222> (412) . . . (412)
<223> Glu or Gln
<222> (460) . . . (460)
<223> Arg or Gln
<222> (461) . . . (461)
<223> Thr or Glu
<222> (552) . . . (552)
<223> Asp or Ser
<222> (556) . . . (556)
<223> Leu or Tyr
<222> (557) . . . (557)
<223> Asp or Ser
<222> (563) . . . (563)
<223> Ser or Asn
<222> (580) . . . (580)
<223> Val or Ile
<222> (588) . . . (588)
<223> Asn or Ser
<222> (664) . . . (664)
<223> Ser or Thr
(Anc82 scaffold)
SEQ ID NO: 140
MAADGYLPDWLEDNLSEGIREWWDLKPGAPKPKANQQKQDDGRGLVLPGYKYLGPENGLD
KGEPVNAADAAALEHDKAYDQQLKAGDNPYLRYNHADAEFQERLQEDTSFGGNLGRAVEQ
AKKRVLEPLGLVEEGAKTAPGKKRPVEQSPQREPDSSXGIGKKGQQPAXKRLNFGQTGDS
ESVPDPQPLGEPPAAPSGVGSNTMAAGGGAPMADNNEGADGVGNSSGNWHCDSTWLGDRV
ITTSTRTWALPTYNNHLYKQISNGTSGGSTNDNTYFGYSTPWGYFDENRFHCHESPRDWQ
RLINNNWGFRPKRLNFKLFNIQVKEVTTNEGTKTIANNLTSTVQVFTDSEYQLPYVLGSA
HQGCLPPFPADVEMIPQYGYLTLNNGSQAVGRSSFYCLEYFPSQMLRTGNNFQFSYTFED
VPFHSSYAHSQSLDRLMNPLIDQYLYYLSRTQTTGGTAGTQTLQFSQAGPSSMANQAKNW
LPGPCYRQQRVSTTTNQNNNSNFAWTGATKYHLNGRDSLVNPGVAMATHKDDEDREFPSS
GVLIFGKQGAGNDNVDYSNVMITXEEEIKTTNPVATEEYGVVATNLQSANTAPQTGTVNS
QGALPGMVWQNRDVYLQGPIWAKIPHTDGNFHPSPLMGGFGLKHPPPQILIKNTPVPADP
PTTFNQAKLNSFITQYSTGQVSVEIEWELQKENSKRWNPEIQYTSNYYKSTNVDFAVNTE
GVYSEPRPIGTRYLTRNL
<222> (158) . . . (158)
<223> Thr or Ser
<222> (169) . . . (169)
<223> Lys or Arg
<222> (564) . . . (564)
<223> Ser or Asn
(Anc83 scaffold)
SEQ ID NO: 141
MAADGYLPDWLEDNLSEGIREWWDLKPGAPKPKANQQKQDDGRGLVLPGYKYLGPENGLD
KGEPVNAADAAALEHDKAYDQQLKAGDNPYLRYNHADAEFQERLQEDTSEGGNLGRAVEQ
AKKRVLEPLGLVEEGAKTAPGKKRPVEQSPQREPDSSXGIGKKGQQPAXKRLNFGOTGDS
ESVPDPQPLGEPPAAPSGVGSNTMAAGGGAPMADNNEGADGVGSSSGNWHCDSTWLGDRV
ITTSTRTWALPTYNNHLYKQISNGTSGGSTNDNTYFGYSTPWGYFDENRFHCHESPRDWQ
RLINNNWGFRPKRLXFKLENIQVKEVTQNEGTKTIANNLTSTIQVFTDSEYQLPYVLGSA
HQGCLPPFPADVEMIPQYGYLTLNNGSQAVGRSSFYCLEYFPSQMLRTGNNEXESYTFED
VPFHSSYAHSQSLDRLMNPLIDQYLYYLSRTQTTGGTAGTQTLQFSQAGPSXMANQAKNW
LPGPCYRQQRVSTTTSQNNNSNFAWTGATKYHLNGRDSLVNPGVAMATHKDDEXRFFPSS
GXLIFGKQGAGKDNVDYSNVMLTSEEEIKTTNPVATEEYGVVADNLQQQNTAPQXGTVNS
QGALPGMVWQNRDVYLQGPIWAKIPHTDGNFHPSPLMGGFGLKHPPPQILIKNTPVPADP
PTTFNQAKLNSFITQYSTGQVSVEIEWELQKENSKRWNPEIQYTSNYYKSTNVDFAVNTE
GVYSEPRPIGTRYLTRNL
<222> (158) . . . (158)
<223> Thr or Ser
<222> (169) . . . (169)
<223> Arg or Lys
<222> (315) . . . (315)
<223> Asn or Ser
<222> (413) . . . (413)
<223> Gln or Glu
<222> (472) . . . (472)
<223> Asn, Thr or Ser
<222> (534) . . . (534)
<223> Asp or Glu
<222> (542) . . . (542)
<223> Ile or Val
<222> (595) . . . (595)
<223> Ile or Val
(Anc84 scaffold)
SEQ ID NO: 142
MAADGYLPDWLEDNLSEGIREWWDLKPGAPKPKANQQKQDDGRGLVLPGYKYLGPENGLD
KGEPVNAADAAALEHDKAYDQQLKAGDNPYLRYNHADAEFQERLQEDTSFGGNLGRAVFQ
AKKRVLEPLGLVEEGAKTAPGKKRPVEPSPQRSPDSSTGIGKKGQQPAXKRLNFGQTGDS
ESVPDPQPIGEPPAAPSGVGSGTMAAGGGAPMADNNEGADGVGSSSGNWHCDSTWLGDRV
ITTSTRTWALPTYNNHLYKQISNGTSGGSTNDNTYFGYSTPWGYFDENRFHCHESPRDWQ
RLINNNWGFRPKRLXFKLENIQVKEVTQNEGTKTIANNLTSTIQVFTDSEYQLPYVLGSA
HQGCLPPFPADVFMIPQYGYLTLNNGSQAVGRSSFYCLEYFPSQMLRTGNNFEFSYTFED
VPFHSSYAHSQSLDRLMNPLIDQYLYYLSRTQSTGGTAGTQQLLFSQAGPSNMSAQAKNW
LPGPCYRQQRVSTTLSQNNNSNFAWTGATKYHLNGRDSLVNPGVAMATHKDDEXRFFPSS
GXLMFGKQGAGKDNVDYSNVMLTSEEEIKTTNPVATEQYGVVADNLQQONTAPIVGAVNS
QGALPGMVWQNRDVYLQGPIWAKIPHTDGNFHPSPLMGGFGLKHPPPQILIKNTPVPADP
PTTFNQAKLNSFITQYSTGQVSVEIEWELQKENSKRWNPEIQYTSNYYKSTNVDFAVNTE
GVYSEPRPIGTRYLTRNL
<222> (169) . . . (169)
<223> Arg or Lys
<222> (315) . . . (315)
<223> Asn or Ser
<222> (534) . . . (534)
<223> Asp or Glu
<222> (542) . . . (542)
<223> Ile or Val
(Anc94 scaffold)
SEQ ID NO: 143
MAADGYLPDWLEDNLSEGIREWWDLKPGAPKPKANQQKODDGRGLVLPGYKYLGPENGLD
KGEPVNAADAAALEHDKAYDQQLKAGDNPYLRYNHADAEFQERLQEDTSFGGNLGRAVFQ
AKKRVLEPLGLVEEGAKTAPGKKRPVEPSPQRSPDSSTGIGKKGQQPAKKRLNFGQTGDS
ESVPDPQPIGEPPAGPSGLGSGTMAAGGGAPMADNNEGADGVGSSSGNWHCDSTWLGDRV
ITTSTRTWALPTYNNHLYKQISNGTSGGSTNDNTYFGYSTPWGYFDENRFHCHESPRDWQ
RLINNNWGFRPKRLNFKLENIQVKEVTQNEGTKTIANNLTSTIQVFTDSEYQLPYVLGSA
HQGCLPPFPADVFMIPQYGYLTLNNGSQAVGRSSFYCLEYFPSQMLRTGNNFEFSYTFED
VPFHSSYAHSQSLDRLMNPLIDQYLYYLSRTQSTGGTAGTQQLLESQAGPXNMSAQAKNW
LPGPCYRQQRVSTTLSQNNNSNFAWTGATKYHLNGRDSLVNPGVAMATHKDDEERFFPSS
GVLMFGKQGAGKDNVDYSSVMLTSEEEIKTTNPVATEQYGVVADNLQQQNTAPIVGAVNS
QGALPGMVWQNRDVYLQGPIWAKIPHTDGNFHPSPLMGGFGLKHPPPQILIKNTPVPADP
PTTFSQAKLASFITQYSTGQVSVEIEWELQKENSKRWNPEIQYTSNYYKSTNVDFAVNTE
GTYSEPRPIGTRYLTRNL
<222> (471) . . . (471)
<223> Ser or Asn
(Anc113 scaffold)
SEQ ID NO: 144
MAADGYLPDWLEDNLSEGIREWWDLKPGAPKPKANQQKQDDGRGLVLPGYKYLGPENGLD
KGEPVNAADAAALEHDKAYDQQLKAGDNPYLRYNHADAEFQERLQEDTSFGGNLGRAVEQ
AKKRVLEPLGLVEEGAKTAPGKKRPVEXSPQRSPDSSTGIGKKGQQPAXKRLNFGQTGDS
ESVPDPQPLGEPPAAPSGVGSGTMAAGGGAPMADNNEGADGVGNASGNWHCDSTWLGDRV
ITTSTRTWALPTYNNHLYKQISSQSAGSTNDNTYFGYSTPWGYFDENRFHCHESPRDWQR
LINNNWGFRPKKLXFKLENIQVKEVTTNDGVTTIANNLTSTVQVESDSEYQLPYVLGSAH
QGCLPPFPADVEMIPQYGYLTLNNGSQSVGRSSFYCLEYFPSQMLRTGNNFEFSYTFEDV
PFHSSYAHSQSLDRLMNPLIDQYLYYLARTQSTTGGTAGNRELQFXQAGPSTMAEQAKNW
LPGPCYRQQRVSKTLDQNNNSNFAWTGATKYHLNGRNSLVNPGVAMATHKDDEDRFFPSS
GVLIFGKTGAANKTTLENVLMTXEEEIKTTNPVATEEYGXVSSNLQSXNTAPQTQTVNSQ
GALPGMVWQNRDVYLQGPIWAKIPHTDGNFHPSPLMGGFGLKHPPPQILIKNTPVPANPP
EVFTPAKFASFITQYSTGQVSVEIEWELQKENSKRWNPEIQYTSNYDKSTNVDFAVDSEG
VYSEPRPIGTRYLTRNL
<222> (148) . . . (148)
<223> Pro or Gln
<222> (169) . . . (169)
<223> Lys or Arg
<222> (314) . . . (314)
<223> Arg or Asn
<222> (466) . . . (466)
<223> Tyr or His
<222> (563) . . . (563)
<223> Asn or Ser
<222> (580) . . . (580)
<223> Val or Ile
<222> (588) . . . (588)
<223> Ala or Ser
(Anc126 scaffold)
SEQ ID NO: 145
MAADGYLPDWLEDNLSEGIREWWDLKPGAPKPKANQQKQDDGRGLVLPGYKYLGPENGLD
KGEPVNAADAAALEHDKAYDQQLKAGDNPYLRYNHADAEFQERLQEDTSFGGNLGRAVFQ
AKKRVLEPLGLVEEGAKTAPGKKRPVEQSPQEPDSSSGIGKXGQQPAXKRLNFGQTGDSE
SVPDPQPLGEPPAAPSGVGSNTMASGGGAPMADNNEGADGVGNXSGNWHCDSTWLGDRVI
TTSTRTWALPTYNNHLYKQISSQSGASNDNHYFGYSTPWGYFDENRFHCHESPRDWQRLI
NNNWGFRPKXLNFKLENIQVKEVTTNDGTTTIANNLTSTVQVFTDSEYQLPYVLGSAHQG
CLPPFPADVEMIPQYGYLTLNNGSQAVGRSSFYCLEYFPSQMLRTGNNFXFSYTFEDVPF
HSSYAHSQSLDRLMNPLIDQYLYYLXRTQTTSGTAQNRELXFSQAGPSSMXNQAKNWLPG
PCYRQQRVSKTANDNNNSNFAWTGATKYHLNGRDSLVNPGPAMASHKDDEDKFFPMSGVL
IFGKQGAGASNVDLDNVMITDEEEIKTTNPVATEQYGTVATNLQSSNTAPATGTVNSQGA
LPGMVWQDRDVYLQGPIWAKIPHTDGHFHPSPLMGGFGLKHPPPQILIKNTPVPANPPTT
FSPAKFASFITQYSTGQVSVEIEWELQKENSKRWNPEIQYTSNYNKSXNVDFTVDINGVY
SEPRPIGTRYLTRNL
<222> (162) . . . (162)
<223> Ser or Thr
<222> (168) . . . (168)
<223> Lys or Arg
<222> (224) . . . (224)
<223> Ala or Ser
<222> (310) . . . (310)
<223> Arg or Lys
<222> (410) . . . (410)
<223> Thr or Gln
<222> (446) . . . (446)
<223> Ser or Asn
<222> (461) . . . (461)
<223> Gln or Leu
<222> (471) . . . (471)
<223> Ala or Ser
<222> (708) . . . (708)
<223> Ala or Thr
(Anc127 scaffold)
SEQ ID NO: 146
MAADGYLPDWLEDNLSEGIREWWDLKPGAPQPKANQQHQDDXRGLVLPGYKYLGPENGLD
KGEPVNEADAAALEHDKAYDQQLKAGDNPYLKYNHADAEFQERLQEDTSFGGNLGRAVEQ
AKKRVLEPLGLVEEAAKTAPGKKRPVEQSPQEPDSSSGIGKSGQQPAXKRLNFGQTGDSE
SVPDPQPLGEPPAAPSGVGSNTMASGGGAPMADNNEGADGVGNSSGNWHCDSTWLGDRVI
TTSTRTWALPTYNNHLYKQISSQSGASNDNHYFGYSTPWGYFDENRFHCHESPRDWQRLI
NNNWGFRPKXLNFKLFNIQVKEVTQNDGTTTIANNLTSTVQVFTDSEYQLPYVLGSAHQG
CLPPFPADVEMIPQYGYLTLNNGSQAVGRSSFYCLEYFPSQMLRTGNNEXFSYTFEDVPF
HSSYAHSQSLDRLMNPLIDQYLYYLXRTQTTSGTTQQSRLXFSQAGPSSMXQQAXNWLPG
PCYRQQRVSKTANDNNNSNFAWTXATKYHLNGRDSLVNPGPAMASHKDDEEKFFPMHGXL
IFGKQGTGASNVDLDNVMITDEEEIRTTNPVATEQYGTVATNLQSSNTAPATGTVNSQGA
LPGMVWQDRDVYLQGPIWAKIPHTDGHFHPSPLMGGFGLKHPPPQILIKNTPVPANPPTT
FSPAKFASFITQYSTGQVSVEIEWELQKENSKRWNPEIQYTSNYNKSVNVDFTVDINGVY
SEPRPIGTRYLTRNL
<222> (42) . . . (42)
<223> Gly or Ser
<222> (168) . . . (168)
<223> Arg or Lys
<222> (310) . . . (310)
<223> Lys or Arg
<222> (410) . . . (410)
<223> Thr or Gln
<222> (446) . . . (446)
<223> Ser or Arg
<222> (461) . . . (461)
<223> Gln or Leu
<222> (471) . . . (471)
<223> Ala or Ser
<222> (475) . . . (475)
<223> Lys or Arg
<222> (504) . . . (504)
<223> Gly or Ala
<222> (539) . . . (539)
<223> Val or Asn
(nucleic acid encoding Anc80 scaffold)
SEQ ID NO: 147
atggctgccgatggttatcttccagattggctcgaggacaacctctctgagggcattcgc
gagtggtgggacttgaaacctggagccccgaaacccaaagccaaccagcaaaagcaggac
gacggccggggtctggtgcttcctggctacaagtacctcggacccttcaacggactcgac
aagggggagcccgtcaacgcggcggacgcagcggccctcgagcacgacaaggcctacgac
cagcagctcaaagcgggtgacaatccgtacctgcggtataaccacgccgacgccgagttt
caggagcgtctgcaagaagatacgtcttttgggggcaacctcgggcgagcagtcttccag
gccaagaagcgggttctcgaacctctcggtctggttgaggaaggcgctaagacggctcct
ggaaagaagagaccggtagagcaatcaccccaggaaccagactcctcttcgggcatcggc
aagaaaggccagcagcccgcgnnnaagagactcaactttgggcagacaggcgactcagag
tcagtgcccgaccctcaaccactcggagaaccccccgcagccccctctggtgtgggatct
aatacaatggcannnggcggtggcgctccaatggcagacaataacgaaggcgccgacgga
gtgggtaacgcctcaggaaattggcattgcgattccacatggctgggcgacagagtcatc
accaccagcacccgaacctgggccctccccacctacaacaaccacctctacaagcaaatc
tccagccaatcgggannnagcaccaacgacaacacctacttcggctacagcaccccctgg
gggtattttgactttaacagattccactgccacttctcaccacgtgactggcagcgactc
atcaacaacaactggggattccggcccaagnnnctcaacttcaagctcttcaacatccag
gtcaaggaggtcacgacgaatgatggcaccacgaccatcgccaataaccttaccagcacg
gttcaggtctttacggactcggaataccagctcccgtacgtcctcggctctgcgcaccag
ggctgcctgcctccgttcccggcggacgtcttcatgattcctcagtacgggtacctgact
ctgaacaatggcagtcaggccgtgggccgttcctccttctactgcctggagtactttcct
tctcaaatgctgagaacgggcaacaactttnnnttcagctacacgtttgaggacgtgcct
tttcacagcagctacgcgcacagccaaagcctggaccggctgatgaaccccctcatcgac
cagtacctgtactacctgtctcggactcagaccacgagtggtaccgcaggaaatcggnnn
ttgcaattttctcaggccgggcctagtagcatggcgaatcaggccaaaaactggctaccc
gggccctgctaccggcagcaacgcgtctccaagacannnaatcaaaataacaacagcaac
tttgcctggaccggtgccaccaagtatcatctgaatggcagagactctctggtaaatccc
ggtcccgctatggcaacccacaaggacgacgaagacaaattttttccgatgagcggagtc
ttaatatttgggaaacagggagctggaaatagcaacgtggaccttgacaacgttatgata
accnnngaggaagaaattaaaaccaccaacccagtggccacagaannntacggcacggtg
gccactaacctgcaatcgnnnaacaccgctcctgctacagggaccgtcaacagtcaagga
gccttacctggcatggtctggcagnnncgggacgtgtacctgcagggtcctatctgggcc
aagattcctcacacggacggacactttcatccctcgccgctgatgggaggctttggactg
aaacacccgcctcctcagatcctgattaagaatacacctgttcccgcgaatcctccaact
accttcagtccagctaagtttgcgtcgttcatcacgcagtacagcaccggacaggtcagc
gtggaaattgaatgggagctgcagaaagaaaacagcaaacgctggaacccagagattcaa
tacacttccaactacaacaaatctacaaatgtggactttgctgttgacacaaatggcgtt
tattctgagcctcgccccatcggcacccgttacctcacccgtaatctg
<222> (502) . . . (504)
<223> This region may encompass “aag” or “aaa”
<222> (613) . . . ( 615)
<223> This region may encompass “gca” or “agc”
<222> (796) . . . (798)
<223> This region may encompass “gca” or “ggc”
<222> (931) . . . (933)
<223> This region may encompass “aga” or “aag”
<222> (1231) . . . (1233)
<223> This region may encompass “gag” or “cag”
<222> (1378) . . . (1380)
<223> This region may encompass “acg” or “gag”
<222> (1477) . . . (1479)
<223> This region may encompass “gcg” or “acc”
<222> (1684) . . . (1686)
<223> This region may encompass “agt” or “aac”
<222> (1726) . . . (1728)
<223> This region may encompass “cag” or “gag”
<222> (1759) . . . (1761)
<223> This region may encompass “tca” or “gcc”
<222> (1825) . . . (1827)
<223> This region may encompass “aac” or “gac”
(nucleic acid encoding Anc81 scaffold)
SEQ ID NO: 148
atggctgccgatggttatcttccagattggctcgaggacaacctctctgagggcattcgc
gagtggtgggacttgaaacctggagccccgaaacccaaagccaaccagcaaaagcaggac
gacggccggggtctggtgcttcctggctacaagtacctcggacccttcaacggactcgac
aagggggagcccgtcaacgcggcggacgcagcggccctcgagcacgacaaggcctacgac
cagcagctcaaagcgggtgacaatccgtacctgcggtataaccacgccgacgccgagttt
caggagcgtctgcaagaagatacgtcttttgggggcaacctcgggcgagcagtcttccag
gccaagaagcgggttctcgaacctctcggtctggttgaggaaggcgctaagacggctcct
ggaaagaagagaccggtagagcaatcaccccaggaaccagactcctctnnnggcatcggc
aagaaaggccagcagcccgcgnnnaagagactcaactttgggcagactggcgactcagag
tcagtgcccgaccctcaaccactcggagaaccccccgcagccccctctggtgtgggatct
aatacaatggctgcaggcggtggcgctccaatggcagacaataacgaaggcgccgacgga
gtgggtaatgcctcaggaaattggcattgcgattccacatggctgggcgacagagtcatc
accaccagcacccgaacctgggccctccccacctacaacaaccacctctacaagcaaatc
tccnnnnnncaatcgggaggaagcaccaacgacaacacctacttcggctacagcaccccc
tgggggtattttgactttaacagattccactgccacttctcaccacgtgactggcagcga
ctcatcaacaacaactggggattccggcccaagnnnctcaacttcaagctcttcaacatc
caggtcaaggaggtcacgacgaatgatggcaccacgaccatcgccaataaccttaccagc
acggttcaggtctttacggactcggaataccagctcccgtacgtcctcggctctgcgcac
cagggctgcctgcctccgttcccggcggacgtcttcatgattcctcagtacgggtacctg
actctgaacaatggcagtcaggccgtgggccgttcctccttctactgcctggagtacttt
ccttctcaaatgctgagaacgggcaacaactttnnnttcagctacacgtttgaggacgtg
ccttttcacagcagctacgcgcacagccaaagcctggaccggctgatgaaccccctcatc
gaccagtacctgtactacctgtctcggactcagaccacgggaggtaccgcaggaaatnnn
nnnttgcaattttctcaggccgggcctagtagcatggcgaatcaggccaaaaactggcta
cccgggccctgctaccggcagcaacgcgtctccaagacaacgaatcaaaataacaacagc
aactttgcctggaccggtgccaccaagtatcatctgaatggcagagactctctggtaaat
cccggtgtcgctatggcaacccacaaggacgacgaagaccgattttttccgtccagcgga
gtcttaatatttgggaaacagggagctggaaatnnnaacgtggacnnnnnnaacgttatg
ataaccnnngaggaagaaattaaaaccaccaacccagtggccacagaagagtacggcnnn
gtggccactaacctgcaatcgnnnaacaccgctcctcaaacagggaccgtcaacagtcaa
ggagccttacctggcatggtctggcagaaccgggacgtgtacctgcagggtcctatctgg
gccaagattcctcacacggacggaaactttcatccctcgccgctgatgggaggctttgga
ctgaaacacccgcctcctcagatcctgattaagaatacacctgttcccgcgaatcctcca
actaccttcnnnccagctaagtttgcgtcgttcatcacgcagtacagcaccggacaggtc
agcgtggaaattgaatgggagctgcagaaagaaaacagcaaacgctggaacccagagatt
caatacacttccaactacaacaaatctacaaatgtggactttgctgttgacacagaaggc
gtttattctgagcctcgccccatcggcacccgttacctcacccgtaatctg
<222> (469) . . . (471)
<223> This region may encompass “acg” or “agc”
<222> (502) . . . (504)
<223> This region may encompass “aaa” or “aag”
<222> (784) . . . (786)
<223> This region may encompass “aac” or “agt”
<222> (787) . . . (789)
<223> This region may encompass “agc” or “cac”
<222> (934) . . . (936)
<223> This region may encompass “aga” or “aag”
<222> (1234) . . . (1236)
<223> This region may encompass “gag” or “cag”
<222> (1378) . . . (1380)
<223> This region may encompass “cgg” or “cag”
<222> (1381) . . . (1383)
<223> This region may encompass “acg” or “gag”
<222> (1654) . . . (1656)
<223> This region may encompass “gac” or “agc”
<222> (1666) . . . (1668)
<223> This region may encompass “ctt” or “tac”
<222> (1669) . . . (1671)
<223> This region may encompass “gac” or “agc”
<222> (1687) . . . (1689)
<223> This region may encompass “agt” or “aac”
<222> (1738) . . . (1740)
<223> This region may encompass “gtg” or “atc”
<222> (1762) . . . (1764)
<223> This region may encompass “gca” or “agc”
<222> (1990) . . . (1992)
<223> This region may encompass “agt” or “acc”
(nucleic acid encoding Anc110 scaffold)
SEQ ID NO: 149
atggctgccgatggttatcttccagattggctcgaggacaacctctctgagggcattcgc
gagtggtgggacttgaaacctggagccccgaaacccaaagccaaccagcaaaagcaggac
gacggccggggtctggtgcttcctggctacaagtacctcggacccttcaacggactcgac
aagggggagcccgtcaacgcggcggacgcagcggccctcgagcacgacaaagcctacgac
cagcagctcaaagcgggtgacaatccgtacctgcggtataatcacgccgacgccgagttt
caggagcgtctgcaagaagatacgtcttttgggggcaacctcgggcgagcagtcttccag
gccaagaagcgggttctcgaacctctcggtctggttgaggaaggcgctaagacggctcct
ggaaagaagaggccggtagagcagtcgccacaagagccagactcctconnnggcatcggc
aagacaggccagcagcccgctnnnaagagactcaattttggtcagactggcgactcagag
tcagtccccgacccacaacctctcggagaacctccagcagccccctcaggtgtgggatct
aatacaatggcttcaggcggtggcgctccaatggcagacaataacgaaggcgccgacgga
gtgggtaattcctcgggaaattggcattgcgattccacatggctgggggacagagtcatc
accaccagcacccgaacctgggccctgcccacctacaacaaccacctctacaagcaaatc
tccaacggcacctcgggaggaagcaccaacgacaacacctattttggctacagcaccccc
tgggggtattttgacttcaacagattccactgtcacttttcaccacgtgactggcaacga
ctcatcaacaacaattggggattccggcccaaaagactcaacttcaagctgttcaacatc
caggtcaaggaagtcacgacgaacgaaggcaccaagaccatcgccaataatctcaccagc
accgtgcaggtctttacggactcggagtaccagttaccgtacgtgctaggatccgctcac
cagggatgtctgcctccgttcccggcggacgtcttcatgattcctcagtacggctattta
actttaaacaatggaagccaagccgtgggacgttcctccttctactgtctggagtatttc
ccatcgcagatgctgagaaccggcaacaactttcagttcagctacaccttcgaggacgtg
cctttccacagcagctacgcgcacagccagagcctggacaggctgatgaatcccctcatc
gaccagtacctgtactacctgtccagaacgcaaacgactggaactnnngggacgcagact
ctgnnnttcagccaagcgggtcctagctcaatggccaaccaggctagaaattgggtgccc
ggaccttgctaccggcagcagcgcgtctccacgacaaccaaccagaacaacaacagcaac
tttgcctggacgggagctnnnaagnnnnnnctgaacggccgagactctctaatgaatccg
ggcgtggcaatggcttcccacaaggatgacgaggaccgcttcttcccttcgagcggggtc
ctgatttttggcaagcaaggagccgggaacgataatgtggattacagcnnngtgatgatt
acaaatgaggaagaaatcaagactaccaaccccgtggccacagaagaatatggagcagtg
gccaccaacnnncagnnngccaatacgcaggcgcagaccggactcgtgcacaaccagggg
gtgcttcccggcatggtgtggcagaatagagacgtgtacctgcagggtcccatctgggcc
aaaattcctcacacggacggcaactttcacccgtctcccctgatgggcggctttggactg
aagcacccgcctcctcaaattctcatcaagaacacaccggttccagcggacccgccgact
accttcaaccaggccaagctgaactctttcatcacgcagtacagcaccggacaggtcagc
gtggaaatcgagtgggagctgcagaaagaaaacagcaaacgctggaatccagagattcaa
tacacttccaactactacaaatctacaaatgtggactttgctgtcaacacggagggggtt
tatagcgagcctcgccccattggcacccgttacctcacccgcaacctgtaa
<222> (469) . . . (471)
<223> This region may encompass “tcg” or “acg”
<222> (502) . . . (504)
<223> This region may encompass “aaa” or “aga”
<222> (1366) . . . (1368)
<223> This region may encompass “gca” or “gga”
<222> (1384) . . . (1386)
<223> This region may encompass “caa” or “gca”
<222> (1519) . . . (1521)
<223> This region may encompass “acc” or “gcc”
<222> (1525) . . . (1527)
<223> This region may encompass “tat” or “ttt”
<222> (1528) . . . (1530)
<223> This region may encompass “cac” or “aaa”
<222> (1669) . . . (1671)
<223> This region may encompass “caa” or “aac”
<222> (1750) . . . (1752)
<223> This region may encompass “aac” or “cac”
<222> (1756) . . . (1758)
<223> This region may encompass “tcc” or “gcc”
(nucleic acid encoding Anc126 scaffold)
SEQ ID NO: 150
atggctgccgatggttatcttccagattggctcgaggacaacctctctgagggcattcgc
gagtggtgggacttgaaacctggagccccgaaacccaaagccaaccagcaaaagcaggac
gacggccggggtctggtgcttcctggctacaagtacctcggacccttcaacggactcgac
aagggggagcccgtcaacgcggcggatgcagcggccctcgagcacgacaaggcctacgac
cagcagctcaaagcgggtgacaatccgtacctgcggtataaccacgccgacgccgagttt
caggagcgtctgcaagaagatacgtcttttgggggcaacctcgggcgagcagtcttccag
gccaagaagagggttctcgaacctcttggtctggttgaggaaggtgctaagacggctcct
ggaaagaaacgtccggtagagcagtcgccacaagagccagactcctcctcgggcattggc
aagnnnggccagcagcccgctnnnaagagactcaattttggtcagactggcgactcagag
tcagtccccgacccacaacctctcggagaacctccagcagccccctctggtgtgggatct
aatacaatggcttcaggcggtggcgcaccaatggcagacaataacgaaggcgccgacgga
gtgggtaatnnntcaggaaattggcattgcgattccacatggctgggcgacagagtcatc
accaccagcacccgaacatgggccttgcccacctataacaaccacctctacaagcaaatc
tccagtcaatcaggggccagcaacgacaaccactacttcggctacagcaccccctggggg
tattttgatttcaacagattccactgccatttctcaccacgtgactggcagcgactcatc
aacaacaattggggattccggcccaagnnnctcaacttcaagctcttcaacatccaagtc
aaggaggtcacgacgaatgatggcaccacgaccatcgctaataaccttaccagcacggtt
caagtcttcacggactcggagtaccagttgccgtacgtcctcggctctgcgcaccagggc
tgcctccctccgttcccggcggacgtgttcatgattccgcagtacggctacctaacgctc
aacaatggcagccaggcagtgggacggtcatccttttactgcctggaatatttcccatcg
cagatgctgagaacgggcaataactttnnnttcagctacaccttcgaggacgtgcctttc
cacagcagctacgcgcacagccagagcctggaccggctgatgaatcctctcatcgaccag
tacctgtattacctgnnnagaactcagactacgtccggaactgcccaaaacagggagttg
nnntttagccaggcgggtccatctagcatgnnnaatcaggccaaaaactggctacctgga
ccctgttaccggcagcagcgcgtttctaaaacagcaaatgacaacaacaacagcaacttt
gcctggactggtgctacaaaatatcaccttaatgggcgtgattctttagtcaaccctggc
cctgctatggcctcacacaaagacgacgaagacaagttctttcccatgagcggtgtcttg
atttttggaaagcagggcgccggagcttcaaacgttgatttggacaatgtcatgatcaca
gacgaagaggaaatcaaaaccactaaccccgtggccaccgaacaatatgggactgtggca
accaatctccagagcagcaacacagcccctgcgaccggaactgtgaattctcagggagcc
ttacctggaatggtgtggcaagacagagacgtatacctgcagggtcctatttgggccaaa
attcctcacacggatggacactttcacccgtctcctctcatgggcggctttggacttaag
cacccgcctcctcagatcctcatcaaaaacacgcctgttcctgcgaatcctccgacaacg
ttttcgcctgcaaagtttgcttcattcatcacccagtattccacaggacaagtgagcgtg
gagattgaatgggagctgcagaaagaaaacagcaaacgctggaatcccgaaatacagtat
acatctaactataataaatctnnnaacgttgatttcactgtggacaccaatggagtttat
agtgagcctcgccccattggcacccgttacctcacccgtaacctg
<222> (484) . . . (486)
<223> This region may encompass “tca” or “aca”
<222> (502) . . . (504)
<223> This region may encompass “aaa” or “aga”
<222> (670) . . . (672)
<223> This region may encompass “gcc” or “tcc”
<222> (928) . . . (930)
<223> This region may encompass “aga” or “aaa”
<222> (1228) . . . (1230)
<223> This region may encompass “acc” or “cag”
<222> (1336) . . . (1338)
<223> This region may encompass “agc” or “aac”
<222> (1381) . . . (1383)
<223> This region may encompass “cag” or “ctg”
<222> (1411) . . . (1413)
<223> This region may encompass “gct” or “tct”
<222> (2122) . . . (2124)
<223> This region may encompass “gcc” or “acc”
(nucleic acid encoding Anc127 scaffold)
SEQ ID NO: 151
atggctgctgacggttatcttccagattggctcgaggacaacctttctgaaggcattcgt
gagtggtgggatctgaaacctggagcccctcaacccaaagcgaaccaacaacaccaggac
gacnnncggggtcttgtgcttccgggttacaaatacctcggaccctttaacggactcgac
aaaggagagccggtcaacgaggcggacgcggcagccctcgaacacgacaaagcttacgac
cagcagctcaaggccggtgacaacccgtacctcaagtacaaccacgccgacgccgagttt
caggagcgtcttcaagaagatacgtcttttgggggcaaccttggcagagcagtcttccag
gccaaaaagagggtccttgagcctcttggtctggttgaggaagcagctaaaacggctcct
ggaaagaagaggcctgtagaacagtctcctcaggaaccggactcatcatctggtattggc
aaatcgggccaacagcctgccnnnaaaagactaaatttcggtcagactggagactcagag
tcagtcccagaccctcaacctctcggagaaccaccagcagccccctcaggtgtgggatct
aatacaatggcttcaggcggtggcgcaccaatggcagacaataacgagggtgccgatgga
gtgggtaattcctcaggaaattggcattgcgattccacatggctgggcgacagagtcatc
accaccagcaccagaacctgggccctgcccacttacaacaaccatctctacaagcaaatc
tccagccaatcaggagcttcaaacgacaaccactactttggctacagcaccccttggggg
tattttgactttaacagattccactgccacttctcaccacgtgactggcagcgactcatt
aacaacaactggggattccggcccaagnnnctcaacttcaagctcttcaacatccaagtt
aaagaggtcacgcagaacgatggcacgacgactattgccaataaccttaccagcacggtt
caagtgtttacggactcggagtatcagctcccgtacgtgctcgggtcggcgcaccaaggc
tgtctcccgccgtttccagcggacgtcttcatgatccctcagtatggatacctcaccctg
aacaacggaagtcaagcggtgggacgctcatccttttactgcctggagtacttcccttcg
cagatgctaaggactggaaataacttcnnnttcagctataccttcgaggatgtacctttt
cacagcagctacgctcacagccagagtttggatcgcttgatgaatcctcttattgatcag
tatctgtactacctgnnnagaacgcaaacaacctctggaacaacccaacaatcacggctg
nnntttagccaggctgggccttcgtctatgnnncagcaggccnnnaattggctacctggg
ccctgctaccggcaacagagagtttcaaagactgctaacgacaacaacaacagtaacttt
gcttggacannngccaccaaatatcatctcaatggccgcgactcgctggtgaatccagga
ccagctatggccagtcacaaggacgatgaagaaaaatttttccctatgcacggcnnncta
atatttggcaaacaagggacaggggcaagtaacgtagatttagataatgtaatgattacg
gatgaagaagagattcgtaccaccaatcctgtggcaacagagcagtatggaactgtggca
actaacttgcagagctcaaatacagctcccgcgactggaactgtcaatagtcagggggcc
ttacctggcatggtgtggcaagatcgtgacgtgtaccttcaaggacctatctgggcaaag
attcctcacacggatggacactttcatccttctcctctgatgggaggctttggactgaaa
catccgcctcctcaaatcttgatcaaaaatactccggtaccggcaaatcctccgacgact
ttcagcccggccaagtttgcttcatttatcactcagtactccactggacaggtcagcgtg
gaaattgagtgggagctacagaaagaaaacagcaaacgttggaatccagagattcagtac
acttccaactacaacaagtctgttaatgtggactttactgtagacactaatggtgtttat
agtgaacctcgccctattggaacccggtatctcacacgaaacttg
<222> (124) . . . (126)
<223> This region may encompass “ggt” or “agt”
<222> (502) . . . (504)
<223> This region may encompass “aga” or “aaa”
<222> (928) . . . (930)
<223> This region may encompass “aaa” or “aga”
<222> (1228) . . . (1230)
<223> This region may encompass “aca” or “cag”
<222> (1336) . . . (1338)
<223> This region may encompass “agc” or “aga”
<222> (1381) . . . (1383)
<223> This region may encompass “caa” or “ctc”
<222> (1411) . . . (1413)
<223> This region may encompass “gct” or “tct”
<222> (1423) . . . (1425)
<223> This region may encompass “aaa” or “aga”
<222> (1510) . . . (1512)
<223> This region may encompass “ggg” or “gcg”
<222> (1615) . . . (1617)
<223> This region may encompass “gtt” or “gac”
(nucleic acid encoding AAV2 vp1)
SEQ ID NO: 152
ATGGCTGCCGATGGTTATCTTCCAGATTGGCTCGAGGACACTCTCTCTGAAGGAATAAGACAGTG
GTGGAAGCTCAAACCTGGCCCACCACCACCAAAGCCCGCAGAGCGGCATAAGGACGACAGCAGGG
GTCTTGTGCTTCCTGGGTACAAGTACCTCGGACCCTTCAACGGACTCGACAAGGGAGAGCCGGTC
AACGAGGCAGACGCCGCGGCCCTCGAGCACGACAAAGCCTACGACCGGCAGCTCGACAGCGGAGA
CAACCCGTACCTCAAGTACAACCACGCCGACGCGGAGTTTCAGGAGCGCCTTAAAGAAGATACGT
CTTTTGGGGGCAACCTCGGACGAGCAGTCTTCCAGGCGAAAAAGAGGGTTCTTGAACCTCTGGGC
CTGGTTGAGGAACCTGTTAAGACGGCTCCGGGAAAAAAGAGGCCGGTAGAGCACTCTCCTGTGGA
GCCAGACTCCTCCTCGGGAACCGGAAAGGCGGGCCAGCAGCCTGCAAGAAAAAGATTGAATTTTG
GTCAGACTGGAGACGCAGACTCAGTACCTGACCCCCAGCCTCTCGGACAGCCACCAGCAGCCCCC
TCTGGTCTGGGAACTAATACGATGGCTACAGGCAGTGGCGCACCAATGGCAGACAATAACGAGGG
CGCCGACGGAGTGGGTAATTCCTCGGGAAATTGGCATTGCGATTCCACATGGATGGGCGACAGAG
TCATCACCACCAGCACCCGAACCTGGGCCCTGCCCACCTACAACAACCACCTCTACAAACAAATT
TCCAGCCAATCAGGAGCCTCGAACGACAATCACTACTTTGGCTACAGCACCCCTTGGGGGTATTT
TGACTTCAACAGATTCCACTGCCACTTTTCACCACGTGACTGGCAAAGACTCATCAACAACAACT
GGGGATTCCGACCCAAGAGACTCAACTTCAAGCTCTTTAACATTCAAGTCAAAGAGGTCACGCAG
AATGACGGTACGACGACGATTGCCAATAACCTTACCAGCACGGTTCAGGTGTTTACTGACTCGGA
GTACCAGCTCCCGTACGTCCTCGGCTCGGCGCATCAAGGATGCCTCCCGCCGTTCCCAGCAGACG
TCTTCATGGTGCCACAGTATGGATACCTCACCCTGAACAACGGGAGTCAGGCAGTAGGACGCTCT
TCATTTTACTGCCTGGAGTACTTTCCTTCTCAGATGCTGCGTACCGGAAACAACTTTACCTTCAG
CTACACTTTTGAGGACGTTCCTTTCCACAGCAGCTACGCTCACAGCCAGAGTCTGGACCGTCTCA
TGAATCCTCTCATCGACCAGTACCTGTATTACTTGAGCAGAACAAACACTCCAAGTGGAACCACC
ACGCAGTCAAGGCTTCAGTTTTCTCAGGCCGGAGCGAGTGACATTCGGGACCAGTCTAGGAACTG
GCTTCCTGGACCCTGTTACCGCCAGCAGCGAGTATCAAAGACATCTGCGGATAACAACAACAGTG
AATACTCGTGGACTGGAGCTACCAAGTACCACCTCAATGGCAGAGACTCTCTGGTGAATCCGGGC
CCGGCCATGGCAAGCCACAAGGACGATGAAGAAAAGTTTTTTCCTCAGAGCGGGGTTCTCATCTT
TGGGAAGCAAGGCTCAGAGAAAACAAATGTGGACATTGAAAAGGTCATGATTACAGACGAAGAGG
AAATCAGGACAACCAATCCCGTGGCTACGGAGCAGTATGGTTCTGTATCTACCAACCTCCAGAGA
GGCAACAGACAAGCAGCTACCGCAGATGTCAACACACAAGGCGTTCTTCCAGGCATGGTCTGGCA
GGACAGAGATGTGTACCTTCAGGGGCCCATCTGGGCAAAGATTCCACACACGGACGGACATTTTC
ACCCCTCTCCCCTCATGGGTGGATTCGGACTTAAACACCCTCCTCCACAGATTCTCATCAAGAAC
ACCCCGGTACCTGCGAATCCTTCGACCACCTTCAGTGCGGCAAAGTTTGCTTCCTTCATCACACA
GTACTCCACGGGACAGGTCAGCGTGGAGATCGAGTGGGAGCTGCAGAAGGAAAACAGCAAACGCT
GGAATCCCGAAATTCAGTACACTTCCAACTACAACAAGTCTGTTAATGTGGACTTTACTGTGGAC
ACTAATGGCGTGTATTCAGAGCCTCGCCCCATTGGCACCAGATACCTGACTCGTAATCTGTAA

What is claimed is:

1. A virus comprising a non-naturally occurring, modified AAV VP1 capsid protein, comprising
an amino acid sequence having at least 95% sequence identity to an amino acid sequence of an unmodified AAV VP1 capsid protein when the amino acid sequence of the modified AAV capsid protein and the amino acid sequence of the unmodified AAV VP1 capsid protein are aligned using a basic local alignment search tool (BLAST) program with default algorithm parameters;
wherein the amino acid sequence of the modified VP1 capsid protein differs from the amino acid sequence of the unmodified VP1 capsid protein in at least one amino acid position selected from the group consisting of amino acid positions Q263, S264, G265, A266, S267, N268, H271, N382, G383, S384, Q385, S446, R471, W502, T503, D528, D529, Q589, K706, and V708, which are numbered relative to AAV2 VP1 capsid protein (SEQ ID NO:1) when SEQ ID NO:1 and the amino acid sequence of the unmodified AAV capsid protein are aligned using the basic local alignment search tool (BLAST) program with default algorithm parameters.

2. The virus of claim 1, wherein the amino acid sequence of the modified VP1 capsid protein comprises at least one amino acid residue selected from the group consisting of 446R, 471A, and 708T.

3. The virus of claim 1, wherein the amino acid sequence of the modified VP1 capsid protein comprises at least two amino acid residues selected from the group consisting of 446R, 471A, and 708T.

4. The virus of claim 1, wherein the amino acid sequence of the modified VP1 capsid protein comprises amino acid residues 446R, 471A, and 708T.

5. The virus of claim 1, wherein the amino acid sequence of the modified VP1 capsid protein comprises at least one amino acid residue selected from the group consisting of 446S, 471S, and 708A.

6. The virus of claim 1, wherein the amino acid sequence of the modified VP1 capsid protein comprises at least two amino acid residues selected from the group consisting of 446S, 471S, and 708A.

7. The virus of claim 1, wherein the amino acid sequence of the modified VP1 capsid protein comprises 446S, 471S, and 708A.

8. The virus of any one of claims 1 to 7, wherein the amino acid sequence of the modified VP1 capsid protein differs from the amino acid sequence of the unmodified VP1 capsid protein only in one or more of the amino acid positions Q263, S264, G265, A266, S267, N268, H271, N382, G383, S384, Q385, S446, R471, W502, T503, D528, D529, Q589, K706, and V708, and in no other amino acid positions in the unmodified VP1 capsid protein.

9. The virus of any one of claims 1 to 8, wherein the unmodified VP1 capsid protein is selected from the group consisting of a VP1 capsid protein from AAV1, AAV2, AAV3, AAV6, AAV7, AAV8, AAV9, rh.10, hu.37, LK-03, AAV5, AAV10, Hu68; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; Anc80DI; AAV1 vp1; AAV2 vp1; AAV9vp1; Anc80; Anc126; Anc127; AAV3; AAV7; AAV8; rh10; hu37; and hu.68.

10. The virus of any one of claims 1 to 9, wherein the non-naturally occurring, modified AAV VP1 capsid protein comprises an amino acid sequence having at least 96%, 97%, 98% or 99% sequence identity to the amino acid sequence of the unmodified AAV VP1 capsid protein when the amino acid sequence of the modified AAV capsid protein and the amino acid sequence of the unmodified AAV VP1 capsid protein are aligned using a basic local alignment search tool (BLAST) program with default algorithm parameters.

11. A modified, assembly-competent recombinant AAV (rAAV), comprising:
VP1, VP2, and VP3 capsid proteins, and
a recombinant nucleic acid vector,
wherein the VP1 capsid protein is the modified VP1 capsid protein of any one of claims 1 to 10.

12. A modified, assembly-competent recombinant AAV (rAAV), comprising:
VP1, VP2, and VP3 capsid proteins; and
a recombinant nucleic acid vector,
wherein at least the VP1 capsid protein is a non-naturally occurring, modified VP1 capsid protein comprising an amino acid sequence having at least 95% sequence identity to an amino acid sequence of an unmodified AAV VP1 capsid protein when the amino acid sequence of the modified AAV capsid protein and the amino acid sequence of the unmodified AAV VP1 capsid protein are aligned using a basic local alignment search tool (BLAST) program with default algorithm parameters, and
wherein the modified VP1 capsid protein differs from the unmodified VP1 capsid protein in comprising a means for altering biodistribution of the modified rAAV following administration of the modified rAAV to a first mammalian subject as compared to biodistribution of an unmodified rAAV following administration of the unmodified rAAV having the unmodified VP1 capsid protein to a second mammalian subject of the same type as the first mammalian subject, wherein the unmodified rAAV comprises VP1, VP2, and VP3 capsid proteins having amino acid sequences identical to those of the modified rAAV except for said means.

13. The modified rAAV of claim 11 or claim 12, wherein the modified rAAV achieves higher transduction of liver cells following administration to a first mammalian subject as compared to transduction of liver cells following administration of the unmodified rAAV comprising the unmodified VP1 capsid protein to a second mammalian subject of the same type as the first mammalian subject.

14. The modified rAAV of any one of claims 11 to 13, wherein the modified rAAV exhibits higher expression in liver cells of an expressible polypeptide encoded by the recombinant nucleic acid vector following administration to a first mammalian subject as compared to expression in liver cells of the expressible polypeptide following administration of an unmodified rAAV comprising the unmodified VP1 capsid protein to a second mammalian subject of the same type as the first mammalian subject.

15. The modified rAAV of claim 11 or claim 12, wherein the modified rAAV achieves lower transduction of liver cells following administration to a first mammalian subject as compared to transduction of liver cells following administration of an unmodified rAAV comprising the unmodified VP1 capsid protein to a second mammalian subject of the same type as the first mammalian subject.

16. The modified rAAV of any one of claim 11, 12, or 15, wherein the modified rAAV exhibits lower expression in liver cells of an expressible polypeptide encoded by the recombinant nucleic acid vector following administration to a first mammalian subject as compared to expression in liver cells of the expressible polypeptide following administration of an unmodified rAAV comprising the unmodified VP1 capsid protein to a second mammalian subject of the same type as the first mammalian subject.

17. The modified rAAV of any one of claims 11 to 16, wherein the modified rAAV has an altered interaction with an AAV receptor (AAVR) expressed on liver cells of the first mammalian subject as compared to an unmodified rAAV comprising the unmodified VP1 capsid protein with an AAVR expressed on liver cells of the second mammalian subject.

18. The modified rAAV of claim 17, wherein the modified rAAV has increased interaction with an AAV receptor (AAVR) expressed on liver cells of the first mammalian subject as compared to an unmodified rAAV comprising the unmodified VP1 capsid protein with an AAVR expressed on liver cells of the second mammalian subject.

19. The modified rAAV of claim 17, wherein the modified rAAV has decreased interaction with an AAV receptor (AAVR) expressed on liver cells of the first mammalian subject as compared to an unmodified rAAV comprising the unmodified VP1 capsid protein with an AAVR expressed on liver cells of the second mammalian subject.

20. The modified rAAV of any one of claims 11 to 19, wherein the first and second mammalian subjects are humans or non-human primates (NHP).

21. The modified rAAV of any one of claims 11 to 20, wherein the administration comprises systemic administration, e.g., intravenous infusion.

22. The modified rAAV of any one of claims 11 to 21, wherein the modified rAAV has lower liver toxicity when administered to a mammalian subject, e.g., a human subject, than an unmodified rAAV comprising the unmodified VP1 capsid protein administered in the same amount by the same route of administration.

23. The modified rAAV of any one of claims 11 to 22, wherein means for altering biodistribution of the modified rAAV following administration of the modified rAAV to a first mammalian subject comprises a mutation at one or more amino acid residues at positions selected from the group consisting of Q263, S264, G265, A266, S267, N268, H271, N382, G383, S384, Q385, S446, R471, W502, T503, D528, D529, Q589, K706, and V708, which are numbered relative to an AAV2 VP1 capsid protein (SEQ ID NO:1) when SEQ ID NO:1 and the amino acid sequence of the unmodified AAV capsid protein are aligned using the basic local alignment search tool (BLAST) program with default algorithm parameters.

24. The modified rAAV of claim 23, wherein the modified VP1 capsid protein comprises at least one amino acid residue selected from the group consisting of 446R, 471A, and 708T.

25. The modified rAAV of claim 23, wherein the modified VP1 capsid protein comprises at least two amino acid residues selected from the group consisting of 446R, 471A, and 708T.

26. The modified rAAV of claim 23, wherein the modified VP1 capsid protein comprises amino acid residues 446R, 471A, and 708T.

27. The modified rAAV of claim 23, wherein the modified VP1 capsid protein comprises at least one amino acid residue selected from the group consisting of 446S, 471S, and 708A.

28. The modified rAAV of claim 23, wherein the modified VP1 capsid protein comprises at least two amino acid residues selected from the group consisting of 446S, 471S, and 708A.

29. The modified rAAV of claim 23, wherein the modified VP1 capsid protein comprises amino acid residues 446S, 471S, and 708A.

30. The modified rAAV of any one of claims 23 to 29, wherein the amino acid sequence of the modified VP1 capsid protein differs from the amino acid sequence of the unmodified VP1 capsid protein only in one or more of the amino acid positions Q263, S264, G265, A266, S267, N268, H271, N382, G383, S384, Q385, S446, R471, W502, T503, D528, D529, Q589, K706, and V708, and in no other amino acid positions in the unmodified VP1 capsid protein.

31. The modified rAAV of any one of claims 11 to 30, wherein the unmodified VP1 capsid protein is selected from the group consisting of a VP1 capsid protein from AAV1, AAV2, AAV3, AAV6, AAV7, AAV8, AAV9, rh.10, hu.37, LK-03, AAV5, AAV10, Hu68; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc110; Anc80DI; AAV1 vp1; AAV2 vp1; AAV9vp1; Anc80; Anc126; Anc127; AAV3; AAV7; AAV8; rh10; hu37; and hu.68.

32. The modified rAAV of any one of claims 11 to 31, wherein the amino acid sequence of the modified VP1 capsid protein is at least 96% identical, 97% identical, 98% identical, or 99% identical to the amino acid sequence of the unmodified VP1 capsid protein.

33. A pharmaceutical composition, comprising:
the modified rAAV of any one of claims 11 to 32, and
a pharmaceutically acceptable carrier.

34. A nucleic acid molecule encoding the modified VP1 capsid protein of any one of claims 1 to 10, or the VP1 protein of the modified rAAV of any one of claims 11 to 32.

35. A vector comprising the nucleic acid molecule of claim 34.

36. An isolated host cell comprising the nucleic acid molecule of claim 34 or the vector of claim 35.

37. A method of altering delivery of an expressible polynucleotide to a target organ of a mammalian subject, e.g., a human patient, as compared to delivery using an rAAV with an unmodified VP1 capsid protein, the method comprising administering a therapeutically effective dose of the modified rAAV of any one of claims 1 to 32 or the pharmaceutical composition of claim 33 to the human patient.

38. The method of claim 37, wherein the expressible nucleic acid is a transgene.

39. The method of claim 37 or claim 38, wherein the modified rAAV exhibits higher transduction of an expressible polypeptide encoded by the recombinant nucleic acid vector into cells of the target organ following administration to a first mammalian subject as compared to transduction into cells of the target organ of the expressible polypeptide following administration of an unmodified rAAV comprising the unmodified VP1 capsid protein to a second corresponding mammalian subject.

40. The method of claim 37 or claim 38, wherein the modified rAAV exhibits lower transduction of an expressible polypeptide encoded by the recombinant nucleic acid vector into cells of the target organ following administration to a first mammalian subject as compared to transduction into cells of the target organ of the expressible polypeptide following administration of an unmodified rAAV comprising the unmodified VP1 capsid protein to a second corresponding mammalian subject.

41. The method of claim 37 or claim 38, wherein the modified rAAV exhibits higher transduction of an expressible polypeptide encoded by the recombinant nucleic acid vector into cells outside of the target organ following administration to a first mammalian subject as compared to transduction into cells outside of the target organ of the expressible polypeptide following administration of an unmodified rAAV comprising the unmodified VP1 capsid protein to a second corresponding mammalian subject.

42. The method of claim 37 or claim 38, wherein the modified rAAV exhibits lower transduction of an expressible polypeptide encoded by the recombinant nucleic acid vector into cells outside of the target organ following administration to a first mammalian subject as compared to transduction into cells outside of the target organ of the expressible polypeptide following administration of an unmodified rAAV comprising the unmodified VP1 capsid protein to a second corresponding mammalian subject.

43. The method of claim 37 or claim 38, wherein the modified rAAV exhibits higher expression of an expressible polypeptide encoded by the recombinant nucleic acid vector in cells in cells of the target organ following administration to a first mammalian subject as compared to expression in cells of the target organ of the expressible polypeptide following administration of an unmodified rAAV comprising the unmodified VP1 capsid protein to a second corresponding mammalian subject.

44. The method of claim 37 or claim 38, wherein the modified rAAV exhibits lower expression of an expressible polypeptide encoded by the recombinant nucleic acid vector in cells of the target organ following administration to a first mammalian subject as compared to expression in cells of the target organ of the expressible polypeptide following administration of an unmodified rAAV comprising the unmodified VP1 capsid protein to a second corresponding mammalian subject.

45. The method of claim 37 or claim 38, wherein the modified rAAV exhibits higher expression in cells outside of the target organ of an expressible polypeptide encoded by the recombinant nucleic acid vector following administration to a first mammalian subject as compared to expression in cells of the target organ of the expressible polypeptide following administration of an unmodified rAAV comprising the unmodified VP1 capsid protein to a second corresponding mammalian subject.

46. The method of claim 37 or claim 38, wherein the modified rAAV exhibits lower expression in cells outside of the target organ of an expressible polypeptide encoded by the recombinant nucleic acid vector following administration to a first mammalian subject as compared to expression in cells of the target organ of the expressible polypeptide following administration of an unmodified rAAV comprising the unmodified VP1 capsid protein to a second corresponding mammalian subject.

47. The method of any one of claims 37 to 46, wherein the target organ is the liver.

48. The method of any one of claims 37 to 47, wherein cells outside the target organ are muscle cells.

49. The method of any one of claims 37 to 47, wherein:
the unmodified AAV is AAV1, AAV8, or AAV9, and the target organ, to which delivery is altered, is the heart;
the unmodified AAV is AAV2, and the target organ, to which delivery is altered, is the kidney;
the unmodified AAV is AAV7, AAV8, AAV9, and the target organ, to which delivery is altered, is the liver;
the unmodified AAV is AAV4, AAV5, AAV6, AAV9, and the target organ, to which delivery is altered, is the lung;
the unmodified AAV is AAV8, and the target organ, to which delivery is altered, is the pancreas;
the unmodified AAV is AAV2, AAV5, AAV8, and the target organ, to which delivery is altered, is the photoreceptor cells of the eye;
the unmodified AAV is AAV1, AAV2, AAV4, AAV5, AAV8, and the target organ, to which delivery is altered, is the Retinal Pigment Epithelium (RPE);
the unmodified AAV is AAV1, AAV6, AAV7, AAV8, AAV9, and the target organ, to which delivery is altered, is the skeletal muscle.

50. The method of any one of claims 37 to 48, wherein the modified rAAV has lower liver toxicity when administered to a mammalian subject, e.g., a human subject, than the same dose of an unmodified rAAV comprising the unmodified VP1 capsid protein administered by the same route of administration.

51. The method of any one of claims 37 to 50, wherein the mammalian subject is a human subject or a non-human primate.

52. A composition for use in the method of any one of claims 37 to 51.
